(19) United States
(12) Patent Application Publication
(10) Pub. No.: US 2026/0061074 A1
(43) Pub. Date: Mar. 5, 2026

(54) GENETIC CONSTRUCTS FOR GENE EDITING

(71) Applicant: Sarepta Therapeutics, Inc., Cambridge, MA (US)

(72) Inventors: Ami M. KABADI, Cambridge, MA (US); Trenton FRISBIE, Cambridge, MA (US); Karen BULAKLAK, Cambridge, MA (US); Chang LIU, Cambridge, MA (US)

(73) Assignee: Sarepta Therapeutics, Inc., Cambridge, MA (US)

(21) Appl. No.: 19/181,618

(22) Filed: Apr. 17, 2025

Related U.S. Application Data
(60) Provisional application No. 63/635,770, filed on Apr. 18, 2024.
(60) Provisional application No. 63/683,285, filed on Aug. 15, 2024.

(51) Int. Cl. A61K 48/00; A61K 9/00; A61K 38/17; A61P 21/00; C12N 9/22; C12N 15/11; C12N 15/86

(52) U.S. Cl. CPC A61K 48/005; A61K 9/0019; A61K 38/1719; A61K 48/0075; A61P 21/00; C12N 9/226; C12N 15/111; C12N 15/86; C12N 2310/20; C12N 2750/14143

(57) ABSTRACT

The present disclosure provides methods and compositions concerning the disclosed genetic constructs, including those that target mutated portions of a dystrophin gene for excision, thereby restoring functional dystrophin protein expression. The disclosure further provides methods and compositions for treating Duchenne muscular dystrophy.

### CROSS REFERENCE TO RELATED APPLICATIONS

This application claims the benefit of U.S. Provisional Appl. No. 63/635,770, filed Apr. 18, 2024 and U.S. Provisional Appl. No. 63/683,285, filed Aug. 15, 2024, the contents of which are hereby incorporated by reference in their entireties.

### REFERENCE TO SEQUENCE LISTING SUBMITTED ELECTRONICALLY

The content of the electronically submitted sequence listing in text format (Name: 4140_1210002_SequenceListing_ST26.xml; Size 356,312 bytes; and Date of Creation: Apr. 16, 2025) filed with the application is incorporated by reference in its entirety.

### FIELD OF DISCLOSURE

The present disclosure relates to the fields of genomic modification via Clustered Regularly Interspaced Short Palindromic Repeats (CRISPR) systems and production of recombinant adeno-associated viruses (rAAV) for use in the treatment of genetic disease.

### BACKGROUND

CRISPR technology is based on bacterial antiviral defense mechanisms and has been harnessed to edit genomic DNA in a targeted manner. However, the full potential of this technology has yet to be realized as much of the putative therapeutic benefit rests on the ability of targeting relevant tissues and cell types in particular disease states and efficiently expressing the components of a gene editing system therein. AAVs represent a particularly useful mode of delivery to particular tissues; however, the packaging capacity of an AAV genome, which is optimally between 4,700 and 4,800 bases, remains a limiting factor for CRISPR gene editing therapies.

As such, further advances in development of AAV regulatory elements that allow for maximal expression of gene products and efficient packaging, while occupying minimal space, are needed. Such developments would facilitate additional space for other components of such a system and would contribute to AAV gene therapies, including CRISPR gene therapies, being more readily available to needy patient populations.

In view of the inevitable lethality and the severe impacts on quality of life associated with a number of genetic diseases, such as Duchenne muscular dystrophy (DMD), it is imperative that broader-spectrum, permanent therapeutic approaches be developed to aid the afflicted population. To this end and given that the association of dystrophin without the domains encoded by exons 45 to 55 with more benign outcomes (see, Poyatos-García, J., et al. (2022). Annals of Neurology, 92 (5), 793-806.), a therapeutic approach that targets the dmd gene mutational hotspot, such that this region is effectively silenced while allowing expression of the remaining portions of the endogenous dystrophin protein, would represent a therapy capable of aiding at least half of the DMD patient population with a single therapeutic intervention.

### BRIEF SUMMARY OF THE DISCLOSURE

The present disclosure concerns methods and compositions for the treatment of genetic disease through expression of one or more gene products, resulting in either reduction or correction of a mutated gene.

In one aspect, the present disclosure encompasses a genetic construct comprising (i) a first inverted terminal repeat (ITR) sequence, (ii) a first promoter, optionally an RNA polymerase II (Pol II)-driven promoter, operably linked to a transgene, (iii) one or more second promoters, optionally RNA polymerase III (Pol III)-driven promoters, operably linked to one or more polynucleotides, wherein the one or more second promoters and the one or more polynucleotides are in a reverse orientation to the first promoter and the transgene, and (iv) a second ITR sequence, wherein said genetic construct is encoded in a single polynucleotide. In some embodiments, the total size of the single polynucleotide is about 4.8 kilobases. In some embodiments, the total size of the single polynucleotide is less than 4.8 kilobases. In some embodiments, the first ITR sequence and/or the second ITR sequence is SEQ ID NO: 99. In some embodiments, the Pol II promoter is selected from any one of a CK8 promoter, an MHCK7 promoter, a Spc512 promoter, or an EFS promoter. In some embodiments, the CK8 promoter is a modified CK8 promoter comprising a nucleotide sequence of SEQ ID NO: 100. In some embodiments, the CK8 promoter is a minimal CK8 promoter comprising a nucleotide sequence of SEQ ID NO: 101. In some embodiments, the transgene is Cas9 or dCas9. In some embodiments, the transgene comprises a nucleotide sequence of SEQ ID NO: 107 (SaCas9). In some embodiments, the transgene comprises a nucleotide sequence of SEQ ID NO: 108 (myospreader Cas9). In some embodiments, the one or more Pol III promoters are selected from any one of a human U6 (hU6) promoter, a murine U6 (mU6) promoter, a 7SK promoter, or an H1 promoter. In some embodiments, the U6 promoter comprises a nucleotide sequence of SEQ ID NO: 103. In some embodiments, the one or more polynucleotides are one or more guide RNAs (gRNAs). In some embodiments, a first gRNA and a second gRNA are encoded within the single polynucleotide. In some embodiments, the first gRNA and the second gRNA target a dystrophin gene.

In some embodiments, the first gRNA targets intron 44 of a dystrophin gene and comprises a sequence selected from SEQ ID NOs: 1-19 and 43-68, wherein the second gRNA targets intron 55 of a dystrophin gene and comprises a sequence selected from SEQ ID NOs: 20-42 and 69-98. In some embodiments, the transgene is Cas9 and i) the first gRNA comprises a sequence of SEQ ID NO: 8 and the second gRNA comprises a sequence of SEQ ID NO: 32; ii) the first gRNA comprises a sequence of SEQ ID NO: 14 and the second gRNA comprises a sequence of SEQ ID NO: 35; iii) the first gRNA comprises a sequence of SEQ ID NO: 4 and the second gRNA comprises a sequence of SEQ ID NO: 27; iv) the first gRNA comprises a sequence of SEQ ID NO: 13 and the second gRNA comprises a sequence of SEQ ID NO: 25; v) the first gRNA comprises a sequence of SEQ ID NO: 11 and the second gRNA comprises a sequence of SEQ ID NO: 26; vi) the first gRNA comprises a sequence of SEQ ID NO: 14 and the second gRNA comprises a sequence of SEQ ID NO: 31; or vii) the first gRNA comprises a sequence of SEQ ID NO: 3 and the second gRNA comprises a sequence of SEQ ID NO: 34.

In some embodiments, the single polynucleotide is at least 95% identical to SEQ ID NO: 133. In some embodiments, the single polynucleotide is at least 99% identical to SEQ ID NO: 133. In some embodiments, the single polynucleotide is identical to SEQ ID NO: 133. In some embodiments, the single polynucleotide is at least 95% identical to SEQ ID NO: 134. In some embodiments, the single polynucleotide is at least 99% identical to SEQ ID NO: 134. In some embodiments, the single polynucleotide is identical to SEQ ID NO: 134. In some embodiments, the single polynucleotide is at least 95% identical SEQ ID NO: 135. In some embodiments, the single polynucleotide is at least 99% identical to SEQ ID NO: 135. In some embodiments, the single polynucleotide is identical to SEQ ID NO: 135. In some embodiments, the single polynucleotide is at least 95% identical SEQ ID NO: 136. In some embodiments, the single polynucleotide is at least 99% identical to SEQ ID NO: 136. In some embodiments, the single polynucleotide is identical to SEQ ID NO: 136. In some embodiments, the single polynucleotide is at least 95% identical SEQ ID NO: 137. In some embodiments, the single polynucleotide is at least 99% identical to SEQ ID NO: 137. In some embodiments, the single polynucleotide is identical to SEQ ID NO: 137. In some embodiments, the single polynucleotide is at least 95% identical SEQ ID NO: 138. In some embodiments, the single polynucleotide is at least 99% identical to SEQ ID NO: 138. In some embodiments, the single polynucleotide is identical to SEQ ID NO: 138. In some embodiments, the single polynucleotide is at least 95% identical SEQ ID NO: 139. In some embodiments, the single polynucleotide is at least 99% identical to SEQ ID NO: 139. In some embodiments, the single polynucleotide is identical to SEQ ID NO: 139. In some embodiments, the single polynucleotide is at least 95% identical SEQ ID NO: 140. In some embodiments, the single polynucleotide is at least 99% identical to SEQ ID NO: 140. In some embodiments, the single polynucleotide is identical to SEQ ID NO: 140.

In one aspect, the disclosure includes a vector capable of expressing any one of the previously described genetic constructs. In some embodiments, the vector is an adeno-associated virus (AAV) vector. In some embodiments, the viral vector is selected from any one of AAV1, AAV2, AAV3, AAV4, AAV5, AAV6, AAV7, AAV8, AAV9, AAV-10, AAV-11, AAV-12, AAV-13, AAVrh.74, MyoAAV, AAVrh74Myo, and a recombinant variant thereof.

In one aspect, the disclosure includes a synthetic promoter comprising the polynucleotide sequence of SEQ ID NO: 100 and a synthetic promoter comprising the polynucleotide sequence of SEQ ID NO: 101. In some embodiments, the promoter drives expression of an operably linked transgene in skeletal muscle or cardiac muscle.

In one aspect, the compositions include a previously described genetic construct, a previously described vector comprising a previously described genetic construct, or a previously described synthetic promoter as a component of a cell. In some embodiments, the cell is a eukaryotic cell. In some embodiments, the cell is a prokaryotic cell. In some embodiments, the cell is a yeast cell. In some embodiments, the cell is an insect cell. In some embodiments, the cell is a mammalian cell. In some embodiments, the cell is a bacterial cell. In some embodiments, the cell is a muscle cell. In some embodiments, the cell is a heart cell. In some embodiments, the cell is a stem cell. In some embodiments, the cell is a satellite cell. In some embodiments, the cell is a liver cell. In some embodiments, the cell is a HeLa cell. In some embodiments, the cell is an HEK-293 cell. In some embodiments, the cell is a PerC.6. In some embodiments, the cell is a Sf9 cell. In another aspect, the compositions include a previously described genetic construct, a previously described vector comprising a previously described genetic construct, or a previously described synthetic promoter as a component of a kit.

In one aspect, the present disclosure includes methods for modifying a mutant gene using a previously described genetic construct. In another aspect, the present disclosure includes a method of genome editing a mutant gene using a previously described genetic construct or a previously described cell. In another aspect, the present disclosure includes a method of treating a subject having a mutant gene using a previously described genetic construct or a previously described cell. In another aspect, the present disclosure includes a method of treating a disease in a patient in need thereof, the method comprising administering to the patient a previously described genetic construct or a previously described cell. In some embodiments, the disease is Duchenne muscular dystrophy. In some embodiments, the disease is Becker muscular dystrophy. In some embodiments, the disease is myotonic dystrophy (DM1), dilated cardiomyopathy, hypertrophic cardiomyopathy, or Facioscapulohumeral muscular dystrophy (FSHD). In some embodiments, the previously described genetic construct or cell is administered to the patient intramuscularly, intravenously, or a combination thereof. In another aspect, the present disclosure includes methods of manufacture of an AAV vector as previously described above, wherein the proportion of full capsids, as determined by SEC-MALS, is at least 25%. In some embodiments, the proportion of full capsids is at least 35%. In some embodiments, the proportion of full capsids is at least 40%. In some embodiments, the AAV vector is AAV.rh74 or a recombinant variant thereof. In some embodiments, the AAV vector is MyoAAV-4E or a recombinant variant thereof.

### BRIEF DESCRIPTION OF DRAWINGS

FIG. 1 illustrates the workflow for selection of gRNA pairs beginning with 3.6 million potential combinations of gRNA pairs capable of deleting exons 45-55 of the DMD gene as part of a CRISPR gene editing system. In silico analysis prioritizing for specificity and chromosomal position reduced this number to approximately 16,000, which were packaged into lentivirus as a pool to create a lentiviral library used to transduce HEK293 cells stably expressing Cas9. Following transduction, the genome DNA from these cells were collected and randomly sheered before hybrid capture to isolate genomic exon 45-55 deletion events, which were resolved by next generation sequencing and computational analysis to map the genomic deletion events back to the correct gRNA pairs.

FIGS. 2A and 2B collectively illustrate that (2A) the frequency of detectable deletion events by gRNA pairs across three screening replicates and (2B) the screen identified a total of 138 gRNA pairs with sufficient deletion efficiency to warrant further consideration when the data is filtered for non-zero editing events.

FIG. 3 illustrates results of a secondary arrayed gene editing experiments with the 138 filtered gRNA pairs described above, measuring splicing of exon 46 to exon 56 in mature transcripts by ddPCR in wild type myoblasts. The highest-performing pairs are indicated in gray and were further analyzed. Quantification of the arrayed screen results is reported in Table 5.

FIGS. 4A, 4B, and 4C collectively illustrate results of validation experiments with the top-performing gRNA pairs comprising the top quartile from the arrayed screen in (4A-4B) delta52 cells and (4C) healthy myoblasts.

FIGS. 5A, 5B, 5C, and 5D collectively illustrate the capability of Cas9 and select gRNA pairs to restore expression of an edited dystrophin protein in diseased cells, wherein no endogenous dystrophin protein is produced due to a mutation within the mutational hotspot.

FIG. 6 illustrates exemplary delivery strategies for gene editing in the adult hDMDΔ52/mdx mouse model of DMD using a dual-vector approach.

FIGS. 7A, 7B, 7C, 7D, and 7E collectively illustrate the varying efficacy of the single-polynucleotide vector approach based upon the relative orientations of the elements within the construct (schematized in 7A), particularly with regard to (7B) Cas9 and (7C) gRNA expression, (7D) dystrophin transcript editing, and (7E) restoration of dystrophin protein expression in murine heart muscle.

FIG. 8 illustrates the comparative editing efficiency of several single-polynucleotide genetic constructs and dual-vector gene editing.

FIGS. 9A, 9B, 9C, and 9D collectively illustrate packaging characteristics of AAV vectors used in (9A, 9B) the dual-vector approach or (9C, 9D) single-polynucleotide approach with historically-configured genetic constructs.

FIGS. 10A, 10B, 10C, and 10D collectively illustrate (10A) descriptions of various optimized single-polynucleotide genetic constructs and the effects of transfecting each construct into HEK-293T cells, including (10B) rate of edited gDNA, (10C) rate of edited dystrophin transcript and (10D) SaCas9 expression.

FIGS. 11A and 11B collectively illustrate viral titers following production of (11A) AAVrh74 and (11B) MyoAAV vectors containing the indicated optimized genetic constructs.

FIGS. 12A and 12B collectively illustrate the proportion of full capsids following production of (12A) AAVrh74 and (12B) MyoAAV-4E vectors containing the indicated optimized genetic constructs.

FIGS. 13A, 13B, and 13C collectively illustrate (13A) schematics of particular genetic construct embodiments, (13B) encapsidation efficiency for these embodiments in MyoAAV-4E vectors, and (13C) dystrophin gene editing efficiency in [A03 Δ52 DMD] myoblasts.

FIG. 14 illustrates relative dose-dependent gene editing of the dystrophin gene within A03 Δ52 DMD myoblasts using the indicated genetic constructs in MyoAAV-4E vectors.

FIGS. 15A, 15B, 15C, 15D, 15E, 15F, 15G, 15H, 15I, 15J, 15K, and 15L collectively illustrate nucleotide sequences of genetic constructs of the present disclosure.

FIGS. 16A, 16B, 16C, 16D, and 16E collectively illustrate (16A) select genetic constructs of the present disclosure and their effects on (16B-16C) targeted transcript deletion, either generally or in specific muscle tissues, (16D) dystrophin protein restoration and (16E) detection of dystrophin in various muscle fibers following administration.

FIGS. 17A, 17B, 17C, 17D, and 17E collectively illustrate (17A) select genetic constructs of the present disclosure containing a myospreader sequence and comparative effects on (17B) Cas9-positive nuclei, (17C) total genome editing, (17D) dystrophin protein restoration following editing, and (17E) detection of dystrophin in muscle fibers following administration.

FIGS. 18A, 18B, and 18C collectively illustrate the impacts of treating DMD model mice with a selected genetic construct on (18A) dystrophin protein restoration in various muscle tissues, (18B) detection of dystrophin in various muscle fibers, and (18C) muscle function following administration.

FIGS. 19A, 19B, 19C, 19D, 19E, 19F, 19G, 19H, and 19I collectively illustrate nucleotide sequences of genetic constructs of the present disclosure.

### DETAILED DESCRIPTION OF THE DISCLOSURE

In some aspects, disclosed herein are compositions and methods for editing a mutational hotspot within the DMD gene. In one aspect, compositions include polypeptides, (e.g., the Cas9 nuclease). In another aspect, the disclosure also provides for polynucleotides (e.g., guide RNAs and/or expression cassettes); polynucleotides encoding said polypeptides; vectors comprising such polynucleotides (e.g., AAV vectors comprising such expression cassettes); methods of making those vectors; recombinant AAV (rAAV) particles comprising such vectors; pharmaceutical compositions comprising the polypeptides, the polynucleotides, the vectors, and/or the rAAV particles disclosed herein; and methods of using the polypeptides, the polynucleotides, the vectors, the rAAV particles, and/or the pharmaceutical compositions disclosed herein.

### 1. Definitions

Unless otherwise defined, all technical and scientific terms used herein have the same meaning as commonly understood by one of ordinary skill in the art. In case of conflict, the present document, including definitions, will control. Unless expressly stated to the contrary herein, any term used in this application, shall have the meaning set forth in this application. While not explicitly defined below, such terms should be interpreted according to their common meaning. The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless explicitly indicated otherwise, all specified embodiments, features, and terms intend to include both the recited embodiment, feature, or term and biological equivalents thereof.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2% and such ranges are included. It is to be understood, although not always explicitly stated, that all numerical designations are preceded by the term “about”. It also is to be understood, although not always explicitly stated, that the reagents described herein are merely exemplary and that equivalents of such are known in the art.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, et al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

As used herein, the terms “about” and/or “approximately” shall mean within an acceptable error range for the particular value as determined by one of ordinary skill in the art, which will depend in part on how the value is measured or determined (i.e., the limitations of the measurement system). For example, “about” can mean within 3 or more than 3 standard deviations, per the practice in the art. Alternatively, “about” can mean a range of up to 20%, preferably up to 10%, more preferably up to 5%, and more preferably still up to 1% of a given value. In certain aspects, the term “about” refers to a range of values that fall within 20%, 19%, 181%, 17%, 16%, 15%, 14%, 13′%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Alternatively, particularly with respect to biological systems or processes, the term can mean within an order of magnitude, preferably within 5-fold, and more preferably within 2-fold, of a value. The terms “Adeno-associated virus” or “AAV” as used interchangeably herein refer to any virus belonging to the Parvoviridae family (genus Dependovirus) that endemically infects humans and some other primate species. AAV is not currently known to cause disease and consequently causes a very mild immune response. In addition to the naturally occurring serotypes of the virus, these terms shall expressly include any and all “recombinant variants” (e.g., engineered versions) of an AAV virus, including, but not limited to, AAVs with RGD insertions (see, e.g., Manini, A., et al. Frontiers in Neurology, 12, 814174 (2022).). Additional non-limiting examples of contemplated recombinant AAV variants include AAVrh.74, MyoAAV variants (e.g., Myo AAV2 and MyoAAV4E), and AAV-MYO variants (see, e.g., Weinmann, J., et al. Nat Commun 11, 5432 (2020)).

The terms “Becker Muscular Dystrophy” or “BMD,” which may be used interchangeably herein, refer to a recessive, X-linked disorder that results in progressive muscle degeneration as a result of aberrant dystrophin function. With onset typically occurring between ages 8 and 15 and with milder symptoms than DMD, BMD patients still ultimately succumb to the disease, most commonly due to heart failure (see Salari, N., et al. Journal of Orthopaedic Surgery and Research, 17 (1), 1-12. (2022)).

The term “Cas9” as used herein Cas9” refers to a Type II CRISPR-Associated nuclease protein that is the active enzyme for a CRISPR-Cas9 system. “nCas9” refers to a Cas9 that has one of the two nuclease domains inactivated, i.e., either the RuvC or HNH domain. nCas9 is capable of cleaving only one strand of target DNA (a “nickase”). The term “Cas9” refers to an RNA-guided double-stranded DNA-binding nuclease protein or nickase protein, or a variant thereof. Herein, “Cas9” refers to both naturally occurring and recombinant Cas9 proteins. A wildtype Cas9 nuclease has two functional domains, e.g., RuvC and HNH, that cut different DNA strands. Cas9 enzymes described herein can comprise a HNH or HNH-like nuclease domain and/or a RuvC or RuvC-like nuclease domain. Cas9 can induce double strand breaks in genomic DNA (e.g., a targeted gene) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species. In specific embodiments, the Cas9 protein is derived from Staphylococcus aureus. In another embodiment, the Cas9 protein comprises a fusion protein comprising a Cas9 enzyme or the active component thereof.

The terms “cardiac muscle” or “heart muscle,” which may be used interchangeably herein, mean a type of involuntary striated muscle tissue found in the walls and histological foundation of the heart, the myocardium. Cardiac muscle is composed of cardiomyocytes or myocardiocytes. These myocardiocytes show striations similar to those on skeletal muscle cells but contain only one, unique nucleus, unlike the multinucleated skeletal cells. In certain embodiments, “cardiac muscle condition” refers to a condition related to the cardiac muscle, such as cardiomyopathy, heart failure, arrhythmia, and inflammatory heart disease.

The term “CK8 promoter” as used herein refers to a synthetic muscle-specific promoter element containing an internal intronic sequence and is capable of driving expression of a gene in skeletal and/or cardiac muscle tissue. In some embodiments, the present disclosure includes modifications of the intronic sequence within the CK8 promoter element (a “modified CK8 promoter”) and/or removal of the intronic sequence (a minimal CK8 promoter”).

The terms “coding sequence” or “encoding nucleic acid” as used herein means the nucleic acids (RNA or DNA molecule) that comprise a nucleotide sequence which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

The terms “complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

The terms “comprise(s),” “include(s),” “having,” “has,” “contain(s),” and variants thereof, as used herein, are intended to be open-ended transitional phrases, terms, or words that do not preclude the possibility of additional acts, structures or components. The singular forms of articles, such as “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The term “directional promoter” refers to two or more promoters that are capable of driving transcription of two separate sequences in both directions. In one embodiment, one promoter drives transcription from 5′ to 3′ and the other promoter drives transcription from 3′ to 5′. In one embodiment, bidirectional promoters are double-strand transcription control elements that can drive expression of at least two separate sequences, for example, coding or non-coding sequences, in opposite directions. Such promoter sequences may be composed of two individual promoter sequences acting in opposite directions, such as one nucleotide sequence linked to the other (complementary) nucleotide sequence, including packaging constructs comprising the two promoters in opposite directions, for example, by hybrid, chimeric or fused sequences comprising the two individual promoter sequences, or at least core sequences thereof, or else by only one transcription regulating sequence that can initiate the transcription in both directions. The two individual promoter sequences, in some embodiments, may be juxtaposed or a linker sequence can be located between the first and second sequences. A promoter sequence may be reversed to be combined with another promoter sequence in the opposite orientation. Genes located on both sides of a bidirectional promoter can be operably linked to a single transcription control sequence or region that drives the transcription in both directions. In other embodiments, the bidirectional promoters are not juxtaposed. For example, one promoter may drive transcription on the 5′ end of a nucleotide fragment, and another promoter may drive transcription from the 3′ end of the same fragment. In another embodiment, a first gene can be operably linked to the bidirectional promoter with or without further regulatory elements, such as a reporter or terminator elements, and a second gene can be operably linked to the bidirectional promoter in the opposite direction and by the complementary promoter sequence, again with or without further regulatory elements.

The terms “donor DNA,” “donor template,” and “repair template” as used interchangeably herein refer to a double-stranded DNA fragment or molecule that includes at least a portion of the gene of interest. The donor DNA may encode a full-functional protein or a partially-functional protein.

The terms “Duchenne Muscular Dystrophy” or “DMD” as used interchangeably herein refers to a recessive, fatal, X-linked disorder that results in muscle degeneration and eventual death. DMD is a common hereditary monogenic disease and occurs in 1 in 3500 males and is the result of inherited or spontaneous mutations in the dmd gene that cause nonsense or frameshift mutations that affect expression of the resultant dystrophin protein. The majority of dystrophin mutations that cause DMD are deletions of exons that disrupt the reading frame and cause premature translation termination in the dystrophin gene. DMD patients typically lose the ability to physically support themselves during childhood or early adolescence and become progressively weaker throughout the teenage years before death in their twenties.

The term “dystrophin” as used herein refers to the protein product of the dmd gene (NCBI Gene ID: 1756; NCBI Protein Accession No.: NP_000100.3; UniProt: P11532). Dystrophin is a rod-shaped cytoplasmic protein, which is a principal component of a protein complex (the dystrophin-associated protein complex or DAPC) that connects cytoskeletal elements of a muscle fiber (i.e., microtubule and actin filaments) to the surrounding extracellular matrix across the cell membrane (sarcolemma). Dystrophin provides structural stability to this complex of the cell membrane, which is responsible for regulating muscle cell integrity and function. The “dystrophin gene” or “dmd gene” as used interchangeably herein is 2.2 megabases in length at locus Xp21 (see, e.g., NCBI Reference NG_012232.1). The primary transcription measures about 2,400 kb with the mature mRNA being about 14 kb. Seventy-nine exons code for the protein, which is composed of more than 3500 amino acids.

The term “efficiency,” as used herein in reference to genome editing, shall mean the rate at which a CRISPR system successfully edits a targeted polynucleotide, as measured by molecular assay, (e.g., ddPCR, Western blotting and/or gene sequencing) and is often expressed as a percentage of an unmodified control. Absolute editing efficiency may vary between two or more CRISPR systems due, wholly or in part, to the choice of a particular genetic sequence target, gRNA structure, chemical modifications of one or more nucleic acids in the system, choice of CRISPR nuclease, CRISPR nuclease amino acid substitutions, among other factors (see, e.g., Li, B., et al. Trends in Pharmacological Sciences, 41 (1), 55-65.) (2020).

The terms “frameshift” or “frameshift mutation,” which may be used interchangeably herein, refer to a type of genetic mutation wherein addition or deletion of one or more nucleotides causes a shift in the codon reading frame in the resultant mRNA, thereby altering the encoded amino acid sequence. Frameshifts may result in, for example, a missense mutation or a nonsense mutation (i.e., introduction of a premature stop codon).

The terms “functional” and “fully functional” as used herein describe protein that has biological activity. A “functional gene” refers to a gene transcribed to mRNA, which is translated to a functional protein.

The term “fusion protein” as used herein refers to a chimeric protein created through the joining of two or more genes that originally coded for separate proteins. The translation of the fusion gene results in a single polypeptide with functional properties derived from each of the original proteins.

The term “gene” as used herein refers to the segment of a DNA molecule that codes for a polypeptide chain (e.g., the coding region). In some embodiments, a gene is positioned by regions immediately preceding, following, and/or intervening the coding region that are involved in producing the polypeptide chain (e.g., regulatory elements such as a promoter, enhancer, polyadenylation sequence, 5′-untranslated region, 3′-untranslated region, or intron).

The terms “genetic construct,” “construct” or “expression cassette” as used herein refer to a nucleic acid molecule generated recombinantly or synthetically, with a series of specified nucleic acid elements that permit transcription of a particular polynucleotide sequence in a competent host cell, such that a particular gene product (e.g., RNA or protein) is expressed. Expression of any gene product may be dependent upon presence of cellular factors or additional gene products from other genetic constructs. Said constructs may be part of a plasmid, viral genome, or nucleic acid fragment. The coding sequence may be DNA or RNA and includes initiation and termination signals operably linked to regulatory elements, such as a promoter and polyadenylation signal capable of directing expression in the cells of the individual to whom the nucleic acid molecule is administered. Typically, such constructs include a polynucleotide to be transcribed, operably linked to a promoter. In some embodiments, an expression cassette comprises a regulatory element operably linked to a polynucleotide sequence encoding a Cas protein or a gRNA. In some embodiments, an expression cassette comprises a nucleotide sequence flanked by a 5′ inverted reverse repeat (ITR) and a 3′ ITR. As used herein, the term “expressible form” refers to genetic constructs that contain the necessary regulatory elements operably linked to a coding sequence that encodes a protein such that when present in the cell of an individual, the coding sequence will be expressed.

The term “genetic disease” as used herein refers to a disease, partially or completely, directly or indirectly, caused by one or more abnormalities in the genome, especially a condition that is present from birth. The abnormality may be a mutation, an insertion or a deletion. The abnormality may affect the coding sequence of the gene or its regulatory sequence. The genetic disease may be, but not limited to DMD, BMD, hemophilia, cystic fibrosis, Huntington's chorea, familial hypercholesterolemia (LDL receptor defect), hepatoblastoma, Wilson's disease, congenital hepatic porphyria, inherited disorders of hepatic metabolism, sickle cell anemia, thalassaemias, xeroderma pigmentosum, Fanconi's anemia, retinitis pigmentosa, ataxia telangiectasia, Bloom's syndrome, retinoblastoma, and Tay-Sachs disease.

The terms “genome editing” or “gene editing” as used herein refer to altering, regulating, or modifying a gene (e.g. a mutant gene), one encoding a truncated protein or non-functional protein), such that a full-length or partially full-length functional protein is expressed or suppressed. Such activity may alternatively be considered “correcting” or “restoring” a mutant gene's functionality and may include replacing or excising an aberrant region of the mutant gene or replacing the entire mutant gene with a copy of the gene that does not have the mutation with a repair mechanism such as homology-directed repair (HDR). Correcting or restoring a mutant gene may also include repairing a frameshift mutation that causes a premature stop codon, an aberrant splice acceptor site, or an aberrant splice donor site, by generating a double stranded break in the gene that is then repaired using non-homologous end joining (NHEJ). NHEJ may add or delete at least one base pair during repair which may restore the proper reading frame and eliminate the premature stop codon. Correcting or restoring a mutant gene may also include deleting a non-essential or aberrant gene segment by the simultaneous action of two nucleases on the same DNA strand. Genome editing additionally refers to modulating expression of a gene as result of altering a genetic sequence (e.g., knocking out a gene, including a mutant gene or a normal gene). Genome editing may be used to treat disease caused by a mutant gene or to enhance repair of tissues by changing expression and/or sequence of a gene product of interest.

The terms “guide RNA” or “gRNA,” which may be used interchangeably herein, refer to one or more RNA molecules, preferably a synthetic RNA molecule, that comprise the RNA component of a CRISPR system (e.g., a CRISPR-Cas9 system) that guides a CRISPR-associated nuclease (e.g., Cas9) to a target polynucleotide or targeted gene. In one embodiment, a gRNA is comprised of a targeting sequence and scaffold sequence. In some embodiments, the gRNA is a single-guide RNA (sgRNA). In some embodiments, the sgRNA is composed of a crRNA and tracrRNA molecule. A sgRNA can be administered or formulated, e.g., as a synthetic RNA, or as a nucleic acid comprising a sequence encoding the gRNA, which is then expressed in one or more target cells. As would be evident to one of ordinary skill in the art, various tools may be used to design and/or optimize the sequence of a gRNA, for example, to increase the specificity and/or precision of genomic editing. In general, an ideal gRNA has a high predicted on-target efficiency and low off-target efficiency based on any of the available web-based tools. Candidate gRNAs may be further assessed by manual inspection and/or experimental screening. Examples of web-based tools include, without limitation, CRISPR seek, CRISPR Design Tool, Cas-OFFinder, E-CRISP, ChopChop, CasOT, CRISPR direct, CRISPOR, BREAKING-CAS, CrispRGold, and CCTop (Safari, et al. Current Pharma. Biotechol. (2017) 18 (13)). Such tools are also described, for example, in PCT Publication No. WO2014093701A1 and Liu, et al., “Computational approached for effective CRISPR guide RNA design and evaluation”, Comput Struct Biotechnol J., 2020; 18:35-44, each of which is incorporated by reference herein in its entirety for all purposes.

The terms “homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including targeted addition of whole genes. If a donor template is provided along with a CRISPR-Cas9 gene editing system, then the cellular machinery will repair the break by homologous recombination, which is enhanced several orders of magnitude in the presence of DNA cleavage. When the homologous DNA piece is absent, non-homologous end joining may take place instead.

The terms “identity,” “identical,” “percent identity,” and/or “percent identical,” as used herein as applicable to one or more particular polynucleotide or amino acid sequences, refer to the proportion of identical residues between a particular reference sequence and another sequence, as calculated by a pairwise alignment using the Needleman-Wunsch algorithm using a generally available alignment program, e.g., the Needle (EMBOSS) program. In cases where the two sequences are of different lengths or the alignment produces one or more staggered ends and the specified region of comparison includes only a single sequence, the residues of the single sequence are included in the denominator but not the numerator for the purposes of calculating identity. When comparing DNA and RNA, thymine (T) and uracil (U) may be considered equivalent. In some embodiments, a claimed sequence includes those that are 100%, 99.5%, 99%, 98%, 97%, 96%, 95%, 94%, 93%, 92%, 91%, and 90% identical to the recited sequence.

The term “mutant gene” or “mutated gene” as used interchangeably herein refers to a gene that has undergone a detectable mutation. A mutant gene has undergone a change, such as the loss, gain, or exchange of genetic material, which affects the normal transmission, expression, and/or functionality of the gene. A “disrupted gene” as used herein refers to a mutant gene that has a mutation that causes a premature stop codon. The disrupted gene product is truncated relative to a full-length undisrupted gene product.

The term “mutational hotspot,” as used herein refers to a segment of genomic DNA that is prone to genetic aberrations. In one example, the mutational hotspot for the dmd gene, refers to an area that includes exons 45 through 55—i.e., the region flanked by intron 44 (e.g., bases 1122695-1371095 of NG_012232.1—Homo sapiens dystrophin (DMD), RefSeqGene (LRG_199) on chromosome X) and intron 55 (e.g., bases 1711938-1832156 of NG_012232.1—Homo sapiens dystrophin (DMD), RefSeqGene (LRG_199) on chromosome X) of the dmd gene. Roughly 45% of all pathogenic dystrophin mutations (e.g., premature stop codons) are located in this genomic region. In-frame deletions of exons 45-55 of the dmd gene result in less pathological symptoms than in DMD and are associated with either very mild Becker phenotypes or even asymptomatic individuals (See, Miyazaki, D., et al. J Hum Genet 54, 127-130 (2009)).

The terms “non-homologous end joining” or “NHEJ” as used herein refer to a cell-mediated DNA double-strand repair process that directly ligates the broken ends without the need for a homologous template. This template-independent re-ligation repair process is stochastic and error-prone, such that random micro-insertions and micro-deletions (indels) are regularly introduced at the DNA breakpoint. This method may be used to intentionally disrupt, delete, or alter the reading frame of targeted polynucleotide sequences in a subject's genome. NHEJ typically uses short homologous DNA sequences called microhomologies to guide repair. These microhomologies are often present in single-stranded overhangs at the end of double-strand breaks. When the overhangs are perfectly compatible, NHEJ usually repairs the break accurately; however, imprecise repair leading to loss of nucleotides may also occur and is much more common when the overhangs are not compatible.

The term “normal gene” as used herein refers to a gene that has not undergone a change, such as a loss, gain, or exchange of genetic material. The normal gene undergoes normal gene transmission and gene expression and is sufficiently functional to not cause symptomatic disease. For the avoidance of doubt, wildtype genes and asymptomatic variants of a wildtype gene, such as those containing single-nucleotide polymorphisms (SNPs), are considered normal genes.

The term “nuclease-mediated NHEJ” as used herein refers to NHEJ that is initiated after a nuclease, such as a Cas9 protein, induces a double-stranded DNA break.

The terms “nucleic acid,” “oligonucleotide” or “polynucleotide” as used herein refer to deoxyribonucleotides or ribonucleotides and polymers thereof in either single- or double-stranded form and complements thereof. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs). Any combinations of bases including uracil, adenine, thymine, cytosine, guanine, inosine, xanthine hypoxanthine, isocytosine and isoguanine are expressly contemplated by this application.

The term “operably linked” as used herein means that expression of a gene is under the control of a promoter or regulatory element with which it is spatially connected. For example, a promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

The term “partially functional” as used herein describes a protein that is encoded by a mutant gene and has less biological activity than a fully functional protein but more than a non-functional protein.

The terms “promoter or “promoter element,” which may be used interchangeably, refer to a nucleotide sequence that assists with controlling expression of a coding sequence. Generally, promoters are located 5′ (i.e., upstream) of the translation start site of a gene. However, in certain embodiments, a promoter element may be located within an intron sequence, or 3′ of the coding sequence. A promoter may be derived from sources including viral, bacterial, fungal, plants, insects, and animals. In some embodiments, one of a plurality of well-characterized promoter elements is used with a vector described herein. Non-limiting examples of well-characterized promoter elements include a SV40 early promoter, a SV40 late promoter, a human U6 (hU6) promoter, a CMV early promoter, a β-actin promoter, and a methyl CpG binding protein 2 (MeCP2) promoter. In some embodiments, the promoter is a constitutive promoter, which drives substantially constant expression of the target protein. In other embodiments, the promoter is tissue-specific promoter, which drives expression of the target protein in response to presence in a particular tissue or cell type. In some embodiments, the promoter is a muscle-specific promoter. Non-limiting examples of muscle-specific promoters include a MHCK7 promoter, a CK8 promoter, and a Spc512 promoter.

A promoter may comprise one or more transcriptional regulatory elements to further enhance expression and/or to alter the spatial expression and/or temporal expression of the same. A promoter may also comprise distal enhancer or repressor elements, which may be located as much as several thousand base pairs from the start site of transcription.

The terms “protospacer,” “targeting sequence” or “crRNA sequence,” which may be used interchangeably refer to a component of a functional gRNA in a CRISPR system that has complementarity to a targeted polynucleotide or targeted gene.

The terms “Protospacer Adjacent Motif” or “PAM,” which may be used interchangeably herein, refer to the region of a targeted gene or targeted polynucleotide sequence that is recognized and bound by a CRISPR-associated (Cas) protein, such as Cas9. In some embodiments, the PAM is no more than 1, 2, 3, 4, 5, 6, 7, 8, 9, 10, 11, 12, 13, 14, 15 or 20 bases from a protospacer sequence. Naturally-occurring Cas9 molecules recognize specific PAM sequences. It is understood that PAMs may be degenerate in nature such that multiple sequences are recognized by a particular protein (e.g., NGG for SpCas9 or NNGRRV/NNGRRT for SaCas9, wherein N means any nucleotide and R means any purine nucleotide, and V means any one of guanine, cytosine, and adenine). In some embodiments, the PAM is NNGRRV. In other embodiments, the PAM is NNGRRT.

The term “regulatory element” as used herein refers to nucleotide sequences, such as promoters, enhancers, terminators, polyadenylation sequences, introns and the like, that provide for the expression of a coding sequence in a cell or otherwise control said expression.

The term “skeletal muscle” as used herein refers to a type of striated muscle, which is under the control of the somatic nervous system and attached to bones by bundles of collagen fibers known as tendons. Skeletal muscle is made up of individual components known as myocytes, sometimes colloquially called “muscle fibers.” Myocytes are formed from the fusion of developmental myoblasts (a type of embryonic progenitor cell that gives rise to a muscle cell) in a process known as myogenesis. These long, cylindrical, multinucleated cells are also called myofibers.

The term “skeletal muscle condition” as used herein refers to a condition related to the skeletal muscle, such as muscular dystrophies, aging, muscle degeneration, wound healing, and muscle weakness or atrophy.

The terms “subject” or “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate—such as, a monkey (e.g., a cynomolgus or rhesus monkey), a chimpanzee, —and a human). In some embodiments, the subject may be a human or a non-human. The subject or patient may be undergoing other forms of treatment.

The terms “target[ed] gene” or “target[ed] polynucleotide” as used herein refer to any nucleotide sequence encoding a known or putative gene product. The target gene may be a mutated gene involved in a genetic disease. In certain embodiments, the target gene is a human dystrophin gene. In certain embodiments, the target gene is a mutant human dystrophin gene.

The term “target region” as used herein refers to the region of the target gene to which the CRISPR/Cas9-based gene editing system is designed to bind and cleave. In some embodiments, the target region is complementary to the protospacer sequence.

The term “transgene” as used herein refers to a protein coding portion of a genetic construct. Such elements may be under the control of a particular promoter or other regulatory elements. In some embodiments, a transgene supplements or replaces a mutant gene product. In some embodiments, a transgene is an effector molecule that facilitates a therapy. Non-limiting examples of transgenes include acid alpha-glucosidase (GAA), Cas9, Cas12f, and microdystrophin.

The term “variant” as used herein encompasses, but is not limited to, proteins (including fusion proteins) which comprise an amino acid sequence that differs from the amino acid sequence of a reference protein by way of one or more substitutions, deletions and/or additions at certain positions within or adjacent to the amino acid sequence of the reference protein. A variant may comprise one or more conservative substitutions in its amino acid sequence as compared to the amino acid sequence of a reference protein. Conservative substitutions may involve, e.g., the substitution of similarly charged or uncharged amino acids. A variant retains the biological activity ascribed to the reference protein. Regarding nucleic acids encoding proteins, a variant may comprise one or more conservative substitutions in its sequence as compared to the sequence of a reference nucleic acid. Conservative nucleic acid substitutions may involve substitution at positions that do not alter the resultant encoded amino acid sequence.

The term “vector” as used herein refers to any vehicle used to transfer a nucleic acid (e.g., a genetic construct encoding a CRISPR-Cas9 system) into a host cell. In some embodiments, a vector includes a replicon, which functions to replicate the vehicle, along with the target nucleic acid. Non-limiting examples of vectors useful for therapeutic purposes include plasmids, phages, cosmids, artificial chromosomes, and viruses, which function as autonomous units of replication in vivo. In some embodiments, the vector is a viral vehicle for introducing a target nucleic acid (e.g., a CRISPR-Cas9 system construct). Many modified eukaryotic viruses useful for genetic construct delivery are known in the art. For example, adeno-associated viruses (AAVs) are particularly well-suited for use in human gene therapy because humans are a natural host for the virus, the native viruses are not known to contribute to any diseases, and the viruses illicit a mild immune response. In certain embodiments, the vector is a lipid nanoparticle.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### 2. Genetic Constructs for Genome Editing of Dystrophin Gene

Provided herein are genetic constructs for genome editing, genomic alteration, and/or altering gene expression of a dystrophin gene. The dystrophin gene may be a human dystrophin gene. The genetic constructs include at least one gRNA that targets a dystrophin gene sequence(s). The at least one gRNA may target a human and/or non-human primate dystrophin gene sequence. The at least one gRNA may be compatible with SaCas9. All gRNAs disclosed herein may be included in a CRISPR/Cas9-based gene editing system, including systems that use SaCas9, to target exons 45 through 55 of the human dystrophin gene. The gRNAs disclosed herein, which may be included in a CRISPR/Cas9-based gene editing system, can cause genomic deletions of the region of exons 45 through 55 of the human dystrophin gene in order to restore expression of functional dystrophin in cells from DMD patients.

a. Dystrophin Gene Biology and Existing DMD Therapies

Dystrophin, the polypeptide product of the dmd gene, is a rod-shaped cytoplasmic protein that is a part of a protein complex that connects the cytoskeleton of a muscle fiber to the surrounding extracellular matrix through the cell membrane and provides structural stability to the dystroglycan complex of the cell membrane. Located on the X chromosome at locus Xp21, the dmd gene is the largest known human gene and contains 79 exons while spanning >2,200 kb (Koenig M et al., (1987). Cell; 50:509-517). The primary transcription measures about 2,400 kb with the mature mRNA being approximately 14 kb. The 79 exons code for the protein, which is over 3500 amino acids.

Normal skeletal muscle tissue express dystrophin, and the absence of dystrophin expression leads to the development of severe and incurable symptoms. Some mutations in the dystrophin gene lead to pre-mature stop codons, resulting in a total lack of dystrophin protein and severe dystrophic phenotype in affected patients (e.g., Duchenne muscular dystrophy or DMD). Some mutations in the dystrophin gene lead to partially functional dystrophin protein and a much milder dystrophic phenotype in affected patients (Becker muscular dystrophy or BMD). Such muscular dystrophies occur at a rate of roughly 1 in 5,000 live male births (Kariyawasam, D., et al. (2022). European Journal of Human Genetics, 30 (12), 1398-1404.). Recent studies have found that a plurality, if not a majority, of these pathogenic mutations occur in a “hotspot” between exons 45 and 55 in the dmd gene (Ousterout, D. G., et al. (2015). Nature Communications, 6 (1), 6244.).

Lack of functional dystrophin causes DMD patients to experience a range of musculoskeletal symptoms, beginning with gradual loss of ambulation before progressing to dilated cardiomyopathy (DCM) and/or respiratory failure, with the latter conditions ultimately becoming fatal before 40 years of age (Danialou, G., et al. (2001). The FASEB Journal, 15 (9), 1655-1657; Verhaert, D., et al. (2011). Circulation: Cardiovascular Imaging, 4 (1), 67-76.). BMD patients, while suffering less loss of ambulation than DMD patients, demonstrate more severe cardiomyopathies, particularly as adults (Esposito, G., & Carsana, A. (2019). Journal of Clinical Medicine, 8 (12), 2151.).

DMD is the result of inherited or spontaneous mutations that cause nonsense or frame shift mutations in the dystrophin gene. Naturally occurring mutations and their consequences are relatively well understood for DMD. In-frame deletions occur in the exon 45-55 regions contained within the rod domain can produce highly functional dystrophin proteins, and many carriers are asymptomatic or display mild symptoms. Furthermore, more than 50% of patients may theoretically be treated by removing exons 45-55. Efforts have been made to restore the disrupted dystrophin reading frame in DMD patients by skipping single non-essential exon(s) (for example, exon 51 skipping) during mRNA splicing to produce internally deleted but functional dystrophin proteins. The deletion of internal dystrophin exon(s) (for example, deletion of exon 51) retains the proper reading frame, albeit with an internally truncated but partially functional dystrophin protein. Additional corrective strategies include supplementation of a dystrophin substitute (e.g., a microdystrophin) to correct DMD.

In certain embodiments, modification of exons 45-55 (such as deletion or excision of exons 45 through 55 by, for example, NHEJ) to restore reading frame ameliorates the phenotype of DMD in subjects, including DMD subjects with deletion mutations. Exons 45 through 55 of a dystrophin gene refers to the 45th exon, 46th exon, 47th exon, 48th exon, 49th exon, 50th exon, 51st exon, 52nd exon, 53rd exon, 54th exon, and the 55th exon of the dystrophin gene. Mutations in one or more of the above-listed exons (45th through 55th exon region) are ideally suited for permanent correction by NHEJ-based genome editing, as disclosed herein.

The presently disclosed genetic constructs are designed to generate deletions in the dystrophin gene. The dystrophin gene may be a human dystrophin gene. In certain embodiments, the vector is configured to form two double-stand breaks (a first double strand break and a second double strand break) in two intronic regions (a first intron and a second intron) flanking a targeted polynucleotide sequence within the dystrophin gene, thereby deleting a segment of the dystrophin gene, comprising the mutational hotspot of the dystrophin gene. A “targeted polynucleotide sequence within a dystrophin gene” includes dystrophin exonic targets positions and dystrophin intronic positions, as described herein. Deletion of a dystrophin exonic target position can optimize the dystrophin sequence of a subject suffering from Duchenne Muscular Dystrophy (DMD) by, for example, increasing the function or activity of the encoded dystrophin protein, and/or result in an improvement in the disease state of the subject. In certain embodiments, excision of the targeted polynucleotide sequence within a dystrophin gene comprising the mutational hotspot restores the proper reading frame. The targeted polynucleotide sequence within a dystrophin gene can comprise one or more exons of the dystrophin gene. In certain embodiments, the dystrophin target position comprises any one of exon 45, exon 46, exon 47, exon 48, exon 49, exon 50, exon 51, exon 52, exon 53, exon 54, and exon 55 of the dystrophin gene (e.g., a human dystrophin gene).

b. CRISPR Biology Overview

In one aspect, compositions disclosed herein, including one or more presently disclosed genetic constructs, can mediate highly efficient gene editing at the exon 45 through exon 55 region of a dystrophin gene. In some embodiments, a presently disclosed genetic construct can restore dystrophin protein expression in cells from DMD patients. Elimination of exons 45 through 55 from the dystrophin transcript by exon skipping can be used to treat approximately 50% of all DMD patients. This class of dystrophin mutations is suited for permanent correction by NHEJ-based genome editing and/or HDR. The genetic constructs described herein have been developed for targeted modification of exon 45 through exon 55 in the human dystrophin gene. A presently disclosed genetic construct may be transfected into human DMD cells and mediate efficient gene modification and conversion to the correct reading frame. Protein restoration may be concomitant with frame restoration and detected in a bulk population of CRISPR/Cas9-based gene editing system-treated cells.

A presently disclosed genetic construct may encode a CRISPR/Cas9-based gene editing system that is specific for a dystrophin gene. “Clustered Regularly Interspaced Short Palindromic Repeats” or “CRISPR,” as used interchangeably herein, refers to loci containing multiple short direct repeats that are found in the genomes of approximately 40% of sequenced bacteria and 90% of sequenced archaea. The CRISPR system is a microbial nuclease system involved in defense against invading phages and plasmids that provides a form of acquired immunity. The CRISPR loci in microbial genomes contain a combination of CRISPR-associated (Cas) genes as well as non-coding RNA elements capable of programming the specificity of the CRISPR-mediated nucleic acid cleavage. In some embodiments, the Cas enzyme comprises a deactivated enzyme (dCas).

Short segments of foreign DNA, called spacers, are incorporated into the genome between CRISPR repeats, and serve as a ‘memory’ of past exposures. Cas9 forms a complex with the 3′ end of the gRNA and the protein-RNA pair recognizes its genomic target by complementary base pairing between the 5′ end of the gRNA sequence and a predefined 20 bp DNA sequence, known as the protospacer. This complex is directed to homologous loci of a pathogenic DNA via regions encoded within the crRNA, protospacers, and protospacer-adjacent motifs (PAMs) within the pathogen genome. The non-coding CRISPR array is transcribed and cleaved within direct repeats into short crRNAs containing individual spacer sequences, which direct Cas nucleases to the target site (protospacer). By simply exchanging the 20 bp recognition sequence of the expressed gRNA, the Cas9 nuclease can be directed to new genomic targets. CRISPR spacers are used to recognize and silence exogenous genetic elements in a manner analogous to RNAi in eukaryotic organisms.

Three classes of CRISPR systems (Types I, II, and Ill effector systems) are known. The Type II effector system carries out targeted DNA double-strand break in four sequential steps, using a single effector enzyme, Cas9, to cleave dsDNA. Compared to the Type I and Type Ill effector systems, which require multiple distinct effectors acting as a complex, the Type II effector system may function in alternative contexts such as eukaryotic cells. The Type II effector system consists of a long pre-crRNA, which is transcribed from the spacer containing CRISPR locus, the Cas9 protein, and a tracrRNA, which is involved in pre-crRNA processing. The tracrRNAs hybridize to the repeat regions separating the spacers of the pre-crRNA, thus initiating dsRNA cleavage by endogenous RNase III. This cleavage is followed by a second cleavage event within each spacer by Cas9, producing mature crRNAs that remain associated with the tracrRNA and Cas9, forming a Cas9: crRNA-tracrRNA complex.

The Cas9: crRNA-tracrRNA complex unwinds the DNA duplex and searches for sequences matching the crRNA to cleave. Target recognition occurs upon detection of complementarity between a protospacer sequence in the target DNA and the remaining spacer sequence in the crRNA. Cas9 mediates cleavage of target DNA if a correct protospacer-adjacent motif (PAM), a short sequence recognized by the Cas9 nuclease that is required for DNA cleavage, is also present at the 3′ end of the protospacer. For protospacer targeting, the sequence must be immediately followed by the PAM.

Different Type II systems have different PAM requirements. For instance, a CRISPR system derived from S. pyogenes may have the PAM sequence for its Cas9 (SpCas9) as 5′-NRG-3′ (where R is either A or G).

A unique capability of the CRISPR/Cas9-based gene editing system is the straightforward ability to simultaneously target multiple distinct genomic loci by co-expressing a single Cas9 protein with two or more sgRNAs. For example, the Streptococcus pyogenes Type II system naturally prefers to use an NGG sequence (where N can be any nucleotide) but also accepts other PAM sequences, such as NAG in other circumstances (Hsu et al., Nature Biotechnology (2013) doi: 10.1038/nbt.2647). Similarly, the Cas9 derived from N. meningitidis (NmCas9) normally recognizes a canonical PAM of NNNNGATT but has activity across a variety of PAMs, including a highly degenerate NNNNGNNN PAM (Esvelt et al. Nature Methods (2013) doi: 10.1038/nmeth2681).

A Cas9 molecule derived from S. aureus recognizes the sequence motif NNGRRT (R=A or G) and directs cleavage of a target nucleic acid sequence 1 to 10, e.g., 3 to 5, bp upstream from that sequence. In certain embodiments, a Cas9 molecule of S. aureus recognizes the PAM sequence motif NNGRRN (R=A or G) and directs cleavage of a target nucleic acid sequence 1 to 10 (e.g., 3 to 5) bp upstream from that sequence. In certain embodiments, a Cas9 molecule derived from S. aureus recognizes the sequence motif NNGRRT (R=A or G) and directs cleavage of a target nucleic acid sequence 1 to 10 (e.g., 3 to 5) bp upstream from that sequence. In certain embodiments, a Cas9 molecule derived from S. aureus recognizes the sequence motif NNGRRV (R=A or G) and directs cleavage of a target nucleic acid sequence 1 to 10 (e.g., 3 to 5) bp upstream from that sequence.

c. CRISPR Gene Editing Systems

In one aspect, Clustered Regularly Interspaced Short Palindromic Repeats (CRISPR) and CRISPR-associated RNA-guided nuclease-related methods, components and compositions of the disclosure (hereafter, CRISPR/Cas systems) minimally require at least one isolated or non-naturally occurring protein component (e.g., a Cas protein) and at least one isolated or non-naturally occurring nucleic acid component (e.g., a guide RNA (gRNA)) to effectuate augmentation of a ‘nucleic acid sequence (e.g., genomic DNA).

In some embodiments, a CRISPR/Cas system effectuates the alteration of a targeted gene or locus in a eukaryotic cell by effecting an alteration of the sequence at a target position (e.g., by creating an insertion or deletion (collectively, an indel) resulting in loss-of-function of (i.e., knocking out) the affected gene or allele; e.g., a nucleotide substitution resulting in a truncation, nonsense mutation, or other type of loss-of-function of an encoded gene product; a of loss-of-function of, for example, an encoded gene product; e.g., loss-of-function of the encoded mRNA or protein by a single nucleotide, double nucleotide, or other frame-shifting deletion, or a deletion resulting in a premature stop codon; or an insertion resulting in a truncation, nonsense mutation, or other type of loss-of-function of an encoded gene product, such as the encoded mRNA or protein; e.g., a single nucleotide, double nucleotide, or other frame-shifting insertions, or an insertion resulting in a premature stop codon.

In some embodiments, a CRISPR/Cas system effectuates the alteration of a targeted gene or locus in a eukaryotic cell by effecting more than one double-strand break. In so doing, an aberrant stretch of genomic DNA (e.g., the mutation hotspot within the dystrophin gene) is permanently excised, such that functionality of the encoded mRNA and/or protein is restored. The present disclosure provides for the alteration (e.g., excision) of exon 45 to exon 55 of the dmd gene in a patient with DMD or BMD by generating two double-strand breaks flanking the targeted polynucleotide sequence within the dmd gene at two target positions.

### (1) CRISPR Nucleases

In one aspect, CRISPR/Cas systems effectuate changes to the sequence of a nucleic acid through nuclease activity. For example, in the case of genomic DNA, the nuclease-guided by a protein-associated exogenous nucleic acid that locates a target position within a targeted gene or locus by sequence complementarity with a portion of the protein-associated nucleic acid (e.g., a protospacer, a CRISPR RNA (crRNA) or a complementary component of a synthetic single guide RNA (sgRNA))-cleaves the genomic DNA upon recognition of particular, nuclease-specific motif called the protospacer adjacent motif (PAM). See generally, Collias, D., & Beisel, C. L. (2021). Nature Communications, 12 (1), 1-12.

Nuclease activity (i.e., cleavage) induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20 (11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences as part of the system along with the nuclease and gRNA. See Bloh, K., & Rivera-Torres, N. (2021). International Journal of Molecular Sciences, 22 (8), 3834.

In general, the minimum requirements of the CRISPR/Cas system will be dependent upon the nuclease (i.e., Cas protein) provided therewith. To this extent, these bacterially derived nucleases have been functionally divided into Types I, III, and V, which all fall into Class 1 and Types II, IV, and VI that are grouped into Class 2.

### a) Class 1 CRISPR Cas Systems

The exact components, compositions, and methods for effectuating a change in a targeted nucleic acid sequence using a Class 1 CRISPR/Cas system will vary but should minimally include: a nuclease (selected from at least Types I, and III), at least one guide RNA selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members.

Cas3 is the prototypical Type I DNA nuclease that functions as the effector protein as part of a larger complex (the Cascade complex comprising Cse1, Cse2,), that is capable of genome editing. See generally He, L., et al. (2020). Genes, 11 (2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule (see, Luo, M. (2015). Nucleic Acids Research, 43 (1), 674-681). As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Biology, 20 (8), 490-507.

Like Type I nucleases, the Type III system relies upon an complex of proteins to effect nucleic acid cleavage. Particularly, Cas10 possesses the nuclease activity to cleave ssDNA in prokaryotes. See, Tamulaitis, G. Trends in Microbiology, 25 (1), 49-61. Interestingly, this CRISPR/Cas system, native to archaea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

### b) Class 2 CRISPR Cas Systems

The exact components, compositions, and methods for effectuating a change in a targeted nucleic acid sequence using a Class 2 CRISPR/Cas system will vary but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members.

Type II nucleases are the best characterized CRISPR/Cas systems, particularly the canonical genomic editing nuclease Cas9 (see Table 4). Multiple Cas9 proteins, derived from various bacterial species, have been isolated. The primary distinction between these nucleases is the PAM, a required recognition site within the targeted dsDNA. After association with a gRNA molecule, the crRNA (or targeting domain of a sgRNA) orients the nuclease at the proper position, but the protein's recognition of the PAM is what induces a cleavage event near that site, resulting in a blunt DSB. have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015), Nature Biotechnology, 33 (2), 179-186. These variants, collectively referred to herein as enhanced specificity Cas9 variants (spCas9), have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally, Naeem, M., et al. (2020), Cells, 9 (7), 1608.

Additional exemplary further engineered variants of canonical Cas proteins (e.g., mutants, chimeras, and include the following (which are hereby incorporated by reference): WO2015035162A2, WO2019126716A1, WO2019126774A1, WO2014093694A1, and WO2014150624A1. For the avoidance of doubt, spCas9 collectively refers to any one of the group consisting of espCas9 (also referred to herein as ESCas9 or esCas9), HFCas9, PECas9, arCas9.

In some instances, one or both of the enzymatic domains of Cas9 are inactivated to generate a deactivated (dCas9) variant. Though lacking the ability to generate double-strand breaks in genomic DNA, dCas9 retains its targeting capabilities when paired with a guide RNA. dCas9, particularly when targeted to regulatory sequences can then impact gene expression (e.g., block an activator or repressor binding site). See, generally Kazi, T. A., & Biswas, S. R. (2021). Progress in Molecular Biology and Translational Science, 178, 99-122. In some embodiments, the CRISPR gene editing system comprises dCas9.

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2c1 (see Table 4), act as part of larger complex of two gRNA-associated nucleases that act on dsDNA as quaternary structure nicking each strand simultaneously (see Zetsche B, et al. Cell. 2015; 163 (3): 759-771; see also Liu L, et al. Mol Cell. 2017; 65 (2): 310-322). Additionally, Cas12b (C2c1) is a highly accurate nuclease with little tolerance for mismatches. See Yang H, et al. Cell. 2016; 167 (7): 1814-1828.e12.

(2) CRISPR guide RNAs

In one aspect, the CRISPR/Cas system of the present disclosure further provides a gRNA molecule (e.g., an isolated or non-naturally occurring RNA molecule) that interacts with a Cas protein. In certain embodiments, the gRNA is an sgRNA, in which the crRNA (i.e., the targeting domain or complementary region) comprises a nucleotide sequence selected from SEQ ID NOs: 1-98 to target a human dmd gene. In certain embodiments, the targeting domain is a crRNA that is provided to a eukaryotic cell with tracrRNA, which acts as a scaffold through interactions with both the crRNA and a Cas protein. In some embodiments, the system is further, optionally, comprised of an oligonucleotide—an HDR template with homology to either side of the target position (see Bloh, K., & Rivera-Torres, N, at 3836).

In some embodiments, the crRNA of the gRNA molecule is configured to orient an associated nuclease such that a cleavage event, (e.g., a double strand break or a locus, thereby facilitating an alteration in the nucleic acid sequence. In some embodiments, the crRNA is 20 nucleotides in length. In some embodiments, the crRNA is 21 nucleotides in length. In some embodiments, the crRNA is 14, 15, 16, 17, 18, 19, 20, 21, 22, 23, 24, 25, 26, 27, 28, 29, or 30 nucleotides in length. In some embodiments, the crRNA orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break may be positioned upstream or downstream of a target position, and either within or upstream a functional domain cluster within the targeted gene.

In certain embodiments, a second gRNA molecule, comprising a second crRNA orients a second associated nuclease such that a cleavage event occurs sufficiently close to a target position, in the targeted gene or locus, thereby facilitating an alteration in the nucleic acid sequence. In an embodiment, the second gRNA molecule targets the same targeted gene or locus as the first gRNA molecule. In other embodiments, the second gRNA molecule targets a different targeted gene or locus as the first gRNA molecule. In some embodiments, the second crRNA is 20 nucleotides in length. In some embodiments, the second crRNA is 14, 15, 16, 17, 18, 19, 20, 21, 22, 23, 24, 25, 26, 27, 28, 29, or 30 nucleotides in length.

In some embodiments, the second crRNA orients the nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. The double-strand or single-strand break, may be positioned upstream or downstream of a target position, and either within or upstream a functional domain cluster within the targeted gene.

In some embodiments, the crRNAs of a first and second gRNA molecules are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the respective target position. In certain embodiments, the first and second gRNA molecules alter the targeted nucleic acid sequences simultaneously. In certain embodiments, the first and second gRNA molecules alter the targeted nucleic acid sequences sequentially. strand break, positioned by the crRNAs of a first and second gRNA molecule, respectively. For example, the crRNAs may orient the associated nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of a target position. In an embodiment, the crRNA of a first and second gRNA molecules are configured to orient associated nucleases such that, for example, two single-strand breaks occurs at the same target position, or within 1, 2, 3, 4, 5, 10, 15, 20, 25, 30, 35, 40, 45, 50 nucleotides of one another, on opposing strands of genomic DNA, thereby essentially approximating a double strand break. In certain embodiments, the first gRNA comprises a crRNA comprising a protospacer sequence selected from any one of SEQ ID NOs: 1-19 and 43-68. In certain embodiments the second gRNA comprises a crRNA comprising a protospacer sequence selected from any one of SEQ ID NOs: 20-42 and 69-98.

In some embodiments, a nucleic acid encodes a crRNA of a first gRNA molecule and a crRNA of a second gRNA molecule comprising protospacer sequences selected from any one of the sequences listed in Table 1, with a PAM selected from any one of the sequences listed in Table 2. In some embodiments, a nucleic acid further encodes a third gRNA molecule. In some embodiments, a nucleic acid further encodes a fourth gRNA molecule.

In certain embodiments, a nucleic acid encodes a crRNA sequence of a first gRNA molecule and a crRNA sequence of a second gRNA molecule, wherein each cRNA comprises a protospacer sequence selected from sequences listed in Table 3. In some embodiments, a nucleic acid encodes a first gRNA molecule comprising a crRNA comprising a protospacer sequence selected from SEQ ID NOs: 1-19, and a second gRNA molecule comprising a crRNA comprising a protospacer sequence selected from SEQ ID NOs: 20-42.

In certain embodiments, a nucleic acid may comprise (a) a sequence encoding a first gRNA molecule, comprising a crRNA with a protospacer that is complementary with a target position in the targeted gene or locus, (b) a sequence encoding a second gRNA molecule, comprising a crRNA with a protospacer that is complementary with a target position in the second targeted gene or locus, and (c) a sequence encoding an RNA-guided nuclease (e.g., Cas9). Optionally, (d) and (e) are sequences encoding a third and fourth gRNA molecule, respectively. In some embodiments, both gRNAs target the same gene or locus. In some embodiments, (a), (b), and (c) are encoded within the same nucleic acid molecule (i.e., the same vector, the same viral vector, the same adeno-associated virus (AAV) vector). In some embodiments, (a) and (b) are encoded within the same nucleic acid molecule (i.e., the same vector, the same viral vector, the same adeno-associated virus (AAV) vector). In some embodiments, (a), (b), and (c) are each encoded within separate nucleic acid molecules (i.e., separate vectors, separate viral vectors, separate adeno-associated virus (AAV) vectors). In some embodiments, (a), (b) and (d) are encoded within the same nucleic acid molecule. In some embodiments, (a), (b) and (e) are encoded within the same nucleic acid molecule. In some embodiments, (a), (b), (d) and (e) are encoded within the same nucleic acid molecule. When more than two gRNAs are used, any combination of (a), (b), (c), (d) and (e) may be encoded within a single or separate nucleic acid molecules.

The CRISPR/Cas9 gene-editing system includes at least one gRNA molecule, for example, two gRNA molecules. The gRNA provides the targeting of a CRISPR/Cas9 gene-editing system. In some embodiments, the gRNA is a sgRNA molecule. The sgRNA is a fusion of two noncoding RNAs: a crRNA and a tracrRNA. The sgRNA may target any desired DNA sequence by exchanging the sequence encoding a protospacer of about 20 bp, which confers targeting specificity through complementary base pairing with the desired DNA target. The gRNA mimics the naturally occurring crRNA: tracrRNA duplex involved in the Type II Effector system. This duplex, which may include, for example, a 42-nucleotide crRNA and a 75-nucleotide tracrRNA, acts as a guide for Cas9 to cleave a targeted polynucleotide. The CRISPR/Cas9 gene-editing system may include at least one gRNA, wherein each gRNA targets a different DNA sequence. The target DNA sequences may be overlapping. The target sequence or protospacer is followed by a PAM sequence at the 3′ end of the protospacer. Different Type II systems have differing PAM requirements. For example, the Streptococcus pyogenes Type II system uses an “NGG” sequence, where “N” can be any nucleotide. In some embodiments, the PAM sequence may be “NGG,”, where “N” can be any nucleotide. In some embodiments, the PAM sequence may be NNGRRT or NNGRRV.

The number of gRNA molecules encoded by a presently disclosed genetic construct (e.g., an AAV vector) can be at least 1 gRNA, at least 2 different gRNAs, at least 3 different gRNAs, at least 4 different gRNAs, at least 5 different gRNAs, at least 6 different gRNAs, at least 7 different gRNAs, at least 8 different gRNAs, at least 9 different gRNAs, at least 10 different gRNAs, at least 11 different 1RNAs, at least 12 different gRNAs, at least 13 different gRNAs, at least 14 different gRNAs, at least i 5 different gRNAs, at least 16 different gRNAs, at least 17 different gRNAs, at least i 8 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNA molecules encoded by a presently disclosed genetic construct can be less than 50 gRNAs, less than 45 different gRNAs, less than 40 different gRNAs, less than 35 different gRNAs, less than 30 different gRNAs, less than 25 different gRNAs, less than 20 different gRNAs, less than 19 different gRNAs, less than 18 different gRNAs, less than 17 different gRNAs, less than 16 different gRNAs, less than 15 different gRNAs, less than 14 different gRNAs, less than 13 different gRNAs less than 12 different gRNAs, less than 11 different gRNAs, less than 10 different gRNAs, less than 9 different gRNAs, less than 8 different gRNAs, less than 7 different gRNAs, less than 6 different gRNAs, less than 5 different gRNAs, less than 4 different gRNAs, or less than 3 different gRNAs. The number of gRNAs encoded by a presently disclosed genetic construct can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs. In certain embodiments, the genetic construct (e.g., an AAV vector) encodes one gRNA molecule, i.e., a first gRNA molecule, and optionally a Cas9 molecule. In certain embodiments, a first genetic construct (e.g., a first AAV vector) encodes one gRNA molecule, i.e., a first gRNA molecule, and optionally a Cas9 molecule, and a second genetic construct (e.g., a second AAV vector) encodes one gRNA molecule, i.e., a second gRNA molecule, and optionally a Cas9 molecule.

The gRNA molecule comprises a targeting domain (also referred to as a targeting sequence or cRNA sequence), which is a complementary polynucleotide sequence of the target DNA sequence followed by a PAM sequence. The gRNA may comprise a “G” at the 5′ end of the targeting domain or complementary polynucleotide sequence. The targeting domain of a gRNA molecule may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair complementary polynucleotide sequence of the target DNA sequence followed by a PAM sequence. The targeting domain of a gRNA molecule may comprise less than a 40 base pair, less than a 35 base pair, less than a 30 base pair, less than a 25 base pair, less than a 20 base pair, less than a 19 base pair, less than a 18 base pair, less than a 17 base pair, less than a 16 base pair, less than a 15 base pair, less than a 14 base pair, less than a 13 base pair, less than a 12 base pair, less than a 11 base pair, or less than a 10 base pair complementary polynucleotide sequence of the target DNA sequence followed by a PAM sequence. In certain embodiments, the targeting domain of a gRNA molecule has 19-25 nucleotides in length. In certain embodiments, the targeting domain of a gRNA molecule is 20 nucleotides in length. In certain embodiments, the targeting domain of a gRNA molecule is 21 nucleotides in length. In certain embodiments, the targeting domain of a gRNA molecule is 22 nucleotides in length. In certain embodiments, the targeting domain of a gRNA molecule is 23 nucleotides in length.

The gRNA may target a region of the dystrophin gene (DMD). In certain embodiments, the gRNA can target at least one of exons, introns, the promoter region, the enhancer region, the transcribed region of the dystrophin gene. In certain embodiments, the gRNA molecule targets intron 44 of the human dystrophin gene. In certain embodiments, the gRNA molecule targets intron 55 of the human dystrophin gene. In some embodiments, a first gRNA and a second gRNA each target an intron of a human dystrophin gene such that exons 45 through 55 are deleted. A gRNA may bind and target a polynucleotide sequence corresponding to SEQ ID NO: 198 or a fragment thereof or a complement thereof. A gRNA may be encoded by a polynucleotide sequence comprising SEQ ID NO: 198 or a fragment thereof or a complement thereof. The targeting sequence of the gRNA may comprise the polynucleotide of SEQ ID NO: 198 or a fragment thereof, such as a 5′ truncation thereof, or a complement thereof. Truncations may be, for example, 1, 2, 3, 4, 5, 6, 7, 8, 9, 10, 11, 12, 13, 14, or 15 nucleotides shorter than SEQ ID NO: 198. In some embodiments, the gRNA may bind and target the polynucleotide of SEQ ID NO: 197. In some embodiments, the gRNA may bind and target a 5′ truncation of the polynucleotide of SEQ ID NO: 198. A gRNA may bind and target a polynucleotide sequence corresponding to SEQ ID NO: 199 or a fragment thereof or a complement thereof. A gRNA may be encoded by a polynucleotide sequence comprising SEQ ID NO: 199 or a fragment thereof or a complement thereof. The targeting sequence of the gRNA may comprise the polynucleotide of SEQ ID NO: 199 or a fragment thereof, such as a 5′ truncation thereof, or a complement thereof. Truncations may be, for example, 1, 2, 3, 4, 5, 6, 7, 8, 9, 10, 11, 12, 13, 14, or 15 nucleotides shorter than SEQ ID NO: 199. In some embodiments, the gRNA may bind and target the polynucleotide of SEQ ID NO: 199. In some embodiments, the gRNA may bind and target a 5′ truncation of the polynucleotide of SEQ ID NO: 199. Single or multiplexed gRNAs can be designed to restore the dystrophin reading frame by targeting the mutational hotspot in exons 45-55 of dystrophin. Following treatment with a presently disclosed vector, dystrophin expression can be restored in one or more DMD patient muscle cells in vitro. In certain embodiments, human dystrophin is detected in vivo following transplantation of genetically corrected patient cells into immunodeficient mice.

### (3) CRISPR Methods Targeting the Dmd Gene

In one aspect, the CRISPR/Cas9 system of the present disclosure comprises at least one Cas protein derived from one or more of the following selected bacterial genera: Corynebacterium, Sutterella, Legionella, Treponema, Filifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevotella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the genomic sequence of a cell from a patient with DMD or BMD) with an RNA-guided nuclease and one or more guide RNAs (gRNAs), resulting in insertion or deletion of one or more nucleotides within the targeted gene product.

In certain embodiments, any region of the dmd gene (e.g., 5′ untranslated region [UTR], exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, exon 49, exon 50, exon 51, exon 52, exon 53, exon 54, exon 55, exon 56, exon 57, exon 58, exon 59, exon 60, exon 61, exon 62, exon 63, exon 64, exon 65, exon 66, exon 67, exon 68, exon 69, exon 70, exon 71 exon 72, exon 73, exon 74, exon 75, exon 76, exon 77, exon, 78, exon 79, intron 1, intron 2, intron 3, intron 4, intron 5, intron 6, intron 7, intron 8, intron 9, intron 10, intron 11, intron 12, intron 13, intron 14, intron 15, intron 16, intron 17, intron 18, intron 19, intron 20, intron 21, intron 22, intron 23, intron 24, intron 25, intron 26, intron 27, intron 28, intron 29, intron 30, intron 31, intron 32, intron 33, intron 34, intron 35, intron 36, intron 37, intron 38, intron 39, intron 40, intron 41, intron 42, intron 43, intron 44, intron 45, intron 46, intron 47, intron 48, intron 49, intron 50, intron 51, intron 52, intron 53, intron 54, intron 55, intron 56, intron 57, intron 58, intron 59, intron 60, intron 61, intron 62, intron 63, intron 64, intron 65, intron 66, intron 67, intron 68, intron 69, intron 70, intron 71 intron 72, intron 73, intron 74, intron 75, intron 76, intron 77, intron 78, any intron/exon junction, the 3′ UTR, or polyadenylation signal) is targeted by an RNA-guided nuclease to alter the gene. In certain embodiments, the targeted gene encodes human dystrophin.

Disclosed herein are methods of genome editing in subject. The genome editing may be in a skeletal muscle and/or cardiac muscle of a subject. The mettlod may comprise administering to the skeletal muscle and/or cardiac muscle of the subject the system or composition for genome editing, as described above. The genome editing may include correcting a mutant gene or inserting a transgene. Correcting the mutant gene may include deleting, rearranging, or replacing the mutant gene. Correcting the mutant gene may include nuclease-mediated NHEJ or HDR.

Disclosed herein are methods of correcting a mutant gene (e.g., a mutant dystrophin gene, e.g., a mutant human dystrophin gene) in a cell and treating a subject suffering from a genetic disease, such as DMD. The method can include administering to a cell or a subject a presently disclosed system or genetic construct (e.g., a vector) or a composition comprising thereof as described above. The method can comprise administering to the skeletal muscle and/or cardiac muscle of the subject the presently disclosed system or genetic construct (e.g., a vector) or a composition comprising the same for genome editing in skeletal muscle and/or cardiac muscle, as described above. Use of the presently disclosed system or genetic construct (e.g., a vector) or a composition comprising the same to deliver the CRISPR/Cas9-based gene editing system to the skeletal muscle or cardiac muscle may restore the expression of a fully-functional or partially functional protein. The CRISPR/Cas9-based gene editing system may be used to introduce site-specific double strand breaks at targeted genomic loci. Site-specific double-strand breaks are created when the CRISPR/Cas9-based gene editing system binds to a target DNA sequences, thereby permitting cleavage of the target DNA. This DNA cleavage may stimulate the natural DNA-repair machinery, leading to, among other possible repair pathways (e.g., homology-directed repair (HDR) or the non-homologous end joining (NHEJ) pathway), ligation without an excised stretch of genomic DNA.

Provided herein is genome editing with a CRISPR/Cas9-based gene editing system without a repair template, which can efficiently correct the reading frame and restore the expression of a functional protein involved in a genetic disease. The disclosed CRISPR/Cas9-based gene editing systems may involve using homology-directed repair or nuclease-mediated non-homologous end joining (NHEJ)-based correction approaches, which enable efficient correction in proliferation-limited primary cell lines that may not be amenable to homologous recombination or selection-based gene correction. This strategy integrates the rapid and robust assembly of active CRISPR/Cas9-based gene editing systems with an efficient gene editing method for the treatment of genetic diseases caused by mutations in nonessential coding regions that cause frameshifts, premature stop codons, aberrant splice donor sites or aberrant splice acceptor sites.

The present disclosure is directed to a method of treating a subject in need thereof. The method comprises administering to a tissue of a subject the presently disclosed system or genetic construct (e.g., a vector) or a composition comprising thereof, as described above. In certain embodiments, the method may comprise administering to the skeletal muscle or cardiac muscle of the subject the presently disclosed system or genetic construct (e.g., a vector) or composition comprising thereof, as described above. In certain embodiments, the method may comprise administering to a vein of the subject the presently disclosed system or genetic construct (e.g., a vector) or composition comprising thereof, as described above. In certain embodiments, the subject is suffering from a skeletal muscle or cardiac muscle condition causing degeneration or weakness or a genetic disease. For example, the subject may be suffering from Duchenne muscular dystrophy (DMD), as described above.

The method, as described above, may be used for correcting the dystrophin gene and recovering full-functional or partially-functional protein expression of said mutated dystrophin gene. In some aspects and embodiments, the disclosure provides a method for reducing the effects (e.g., clinical symptoms/indications) of DMD in a patient. In some aspects and embodiments, the disclosure provides a method for treating DMD in a patient. In some aspects and embodiments, the disclosure provides a method for preventing DMD in a patient. In some aspects and embodiments the disclosure provides a method for preventing further progression of DMD in a patient.

### (4) Pharmaceutical Compositions Targeting the Dmd Gene

Further disclosed herein are DNA targeting compositions that comprise genetic constructs. The DNA targeting compositions include at least one gRNA molecule (for example, two gRNA molecules) that targets a dystrophin gene (for example, a human dystrophin gene), as described above. The at least one gRNA molecule can bind and recognize a target region. The target regions can be chosen immediately upstream of possible out-of-frame stop codons, such that insertions or deletions during the repair process restore the dystrophin reading frame by frame conversion. Target regions can also be splice acceptor sites or splice donor sites, such that insertions or deletions during the repair process disrupt splicing and restore the dystrophin reading frame by splice site disruption and exon exclusion. Target regions can also be aberrant stop codons, such that insertions or deletions during the repair process restore the dystrophin reading frame by eliminating or disrupting the stop codon. In some embodiments, any DNA abnormalities (for example, those listed above) occur with the mutational hotspot of a human dystrophin gene, such that excision of the mutational hotspot restores dystrophin functionality.

In certain embodiments, the presently disclosed DNA targeting composition includes a first gRNA and a second gRNA. The first gRNA molecule and the second gRNA molecule may bind or target a polynucleotide of SEQ ID NO: 198 and SEQ ID NO: 199, respectively, or a truncation or a complement thereof. The first gRNA molecule and the second gRNA molecule may comprise a polynucleotide corresponding to any one of SEQ ID NOs: 1-98, or a truncation or a complement thereof.

The deletion efficiency of the presently disclosed vectors can be related to the deletion size, i.e., the size of the segment deleted by the vectors. In certain embodiments, the length or size of specific deletions is determined by the distance between the PAM sequences in the gene being targeted (e.g., a dystrophin gene). In certain embodiments, a specific deletion of a segment of the dystrophin gene, which is defined in terms of its length and a sequence it comprises (e.g., exon 51), is the result of breaks made adjacent to specific PAM sequences within the target gene (e.g., a dystrophin gene).

In certain embodiments, the deletion size is about 50 to about 2,000 base pairs (bp), e.g., about 50 to about 1999 bp, about 50 to about 1900 bp, about 50 to about 1800 bp, about 50 to about 1700 bp, about 50 to about 1650 bp, about 50 to about 1600 bp, about 50 to about 1500 bp, about 50 to about 1400 bp, about 50 to about 1300 bp, about 50 to about 1200 bp, about 50 to about 1150 bp, about 50 to about 1100 bp, about 50 to about 1000 bp, about 50 to about 900 bp, about 50 to about 850 bp, about 50 to about 800 bp, about 50 to about 750 bp, about 50 to about 700 bp, about 50 to about 600 bp, about 50 to about 500 bp, about 50 to about 400 bp, about 50 to about 350 bp, about 50 to about 300 bp, about 50 to about 250 bp, about 50 to about 200 bp, about 50 to about 150 bp, about 50 to about 100 bp, about 100 to about 1999 bp, about 100 to about 1900 bp, about 100 to about 1800 bp, about 100 to about 1700 bp, about 100 to about 1650 bp, about 100 to about 1600 bp, about 100 to about 1500 bp, about 100 to about 1400 bp, about 100 to about 1300 bp, about 100 to about 1200 bp, about 100 to about 1150 bp, about 100 to about 1100 bp, about 100 to about 1000 bp, about 100 to about 900 bp, about 100 to about 850 bp, about 100 to about 800 bp, about 100 to about 750 bp, about 100 to about 700 bp, about 100 to about 600 bp, about 100 to about 1000 bp, about 100 to about 400 bp, about 100 to about 350 bp, about 100 to about 300 bp, about 100 to about 250 bp, about 100 to about 200 bp, about 100 to about 150 bp, about 200 to about 1999 bp, about 200 to about 1900 bp, about 200 to about 1800 bp, about 200 to about 1700 bp, about 200 to about 1650 bp, about 200 to about 1600 bp, about 200 to about 1500 bp, about 200 to about 1400 bp, about 200 to about 1300 bp, about 200 to about 1200 bp, about 200 to about 1150 bp, about 200 to about 1100 bp, about 200 to about 1000 bp, about 200 to about 900 bp, about 200 to about 850 bp, about 200 to about 800 bp, about 200 to about 750 bp, about 200 to about 700 bp, about 200 to about 600 bp, about 200 to about 2000 bp, about 200 to about 400 bp, about 200 to about 350 bp, about 200 to about 300 bp, about 200 to about 250 bp, about 300 to about 1999 bp, about 300 to about 1900 bp, about 300 to about 1800 bp, about 300 to about 1700 bp, about 300 to about 1650 bp, about 300 to about 1600 bp, about 300 to about 1500 bp, about 300 to about 1400 bp, about 300 to about 1300 bp, about 300 to about 1200 bp, about 300 to about 1150 bp, about 300 to about 1100 bp, about 300 to about 1000 bp, about 300 to about 900 bp, about 300 to about 850 bp, about 300 to about 800 bp, about 300 to about 750 bp, about 300 to about 700 bp, about 300 to about 600 bp, about 300 to about 3000 bp, about 300 to about 400 bp, or about 300 to about 350 bp. In certain embodiments, the deletion size can be about 118 base pairs, about 233 base pairs, about 326 base pairs, about 766 base pairs, about 805 base pairs, or about 1611 base pairs.

Disclosed herein is a genetic construct or a composition thereof for genome editing a target gene in a subject, such as, for example, a target gene in skeletal muscle and/or cardiac muscle of a subject. The genetic construct may be a vector. The vector may be a modified AAV vector. The composition may include a polynucleotide sequence encoding a CRISPR/Cas9-based gene editing system. The composition may deliver active forms of CRISPR/Cas9-based gene editing systems to skeletal muscle or cardiac muscle. The presently disclosed genetic constructs can be used in correcting or reducing the effects of mutations in the dystrophin gene involved in genetic diseases and/or other skeletal or cardiac muscle conditions, such as, for example, DMD. These compositions may further comprise a donor DNA or a transgene. These compositions may be used in genome editing, genome engineering, and correcting or reducing the effects of mutations in genes involved in genetic diseases and/or other skeletal and/or cardiac muscle conditions.

A CRISPR/Cas9-based gene editing system specific for dystrophin gene is disclosed herein. The CRISPR/Cas9-based gene editing system may include Cas9 and at least one gRNA to target the dystrophin gene. The CRISPR/Cas9-based gene editing system may bind and recognize a target region. The target regions may be chosen immediately upstream of possible out-of-frame stop codons such that insertions or deletions during the repair process restore the dystrophin reading frame by frame conversion. Target regions may also be splice acceptor sites or splice donor sites, such that insertions or deletions during the repair process disrupt splicing and restore the dystrophin reading frame by splice site disruption and exon exclusion. Target regions may also be aberrant stop codons such that insertions or deletions during the repair process restore the dystrophin reading frame by eliminating or disrupting the stop codon. Target regions may include an intron of the dystrophin gene. Target regions may include an exon of the dystrophin gene.

The composition may also include a viral delivery system. In certain embodiments, the vector is an adeno-associated virus (AAV) vector. The AAV vector is a small virus belonging to the genus Dependovirus of the Parvoviridae family that infects humans and some other primate species. AAV vectors may be used to deliver CRISPR/Cas9-based gene editing systems using various construct configurations. For example, AAV vectors may deliver Cas9 and gRNA expression cassettes on separate vectors or on the same vector. Alternatively, if the small Cas9 proteins, derived from species such as Staphylococcus aureus or Neisseria meningitidis, are used, then both the Cas9 and up to two gRNA expression cassettes may be combined in a single AAV vector within the 4.7 kb packaging limit.

In certain embodiments, the AAV vector is a recombinant AAV variant vector. The recombinant AAV variant vector may have enhanced cardiac and skeletal muscle tissue tropism. The recombinant AAV variant vector may be capable of delivering and expressing the CRISPR/Cas9-based gene editing system in the cell of a mammal. For example, the recombinant AAV variant vector may be an AAV-SASTG vector (Piacentino et al. (2012) Human Gene Therapy 23:635-646). The recombinant AAV variant vector may deliver nucleases to skeletal and cardiac muscle in vivo. The recombinant AAV variant vector may be based on one or more of several capsid types, including AAV1, AAV2, AAV5, AAV6, AAV8, AAV9 and AAVrh74. The recombinant AAV variant vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy (2012) 12:139-151). The recombinant AAV variant vector may be AAV218G9 (Shen et al., J. Biol. Chem. (2013) 288: 28814-28823). The AAV vector may be AAVrh74.

The compositions, as described above, may comprise one or more genetic constructs that encode the CRISPR/Cas9-based gene editing system, as disclosed herein. The genetic construct, such as a plasmid, may comprise a nucleic acid that encodes the CRISPR/Cas9-based gene editing system, such as the Cas9 protein and/or Cas9 fusion proteins and/or at least one of the gRNAs. The compositions, as described above, may comprise genetic constructs that encode the modified AAV vector and a nucleic acid sequence that encodes the CRISPR/Cas9-based gene editing system, as disclosed herein. The genetic construct, such as a plasmid, may comprise a nucleic acid that encodes the CRISPR/Cas9-based gene editing system. The compositions, as described above, may comprise genetic constructs that encode the modified lentiviral vector, as disclosed herein.

The genetic construct, such as a recombinant plasmid or recombinant viral particle, may comprise a nucleic acid that encodes the Cas9-fusion protein and at least one gRNA. In some embodiments, the genetic construct may comprise a nucleic acid that encodes the Cas9-fusion protein and at least two different gRNAs. In some embodiments, the genetic construct may comprise a nucleic acid that encodes the Cas9-fusion protein and more than two different gRNAs. In some embodiments, the genetic construct may comprise a promoter that operably linked to the nucleotide sequence encoding the at least one gRNA molecule and/or a Cas9 molecule. In some embodiments, the promoter is operably linked to the nucleotide sequence encoding a first gRNA molecule, a second gRNA molecule, and/or a Cas9 molecule. The genetic construct may be present in the cell as a functioning extrachromosomal molecule. The genetic construct may be a linear minichromosome including centromere, telomeres or plasmids or cosmids.

The genetic construct may also be part of a genome of a recombinant viral vector, including recombinant lentivirus, recombinant adenovirus, and recombinant adenovirus associated virus. The genetic construct may be part of the genetic material in attenuated live microorganisms or recombinant microbial vectors which live in cells. The genetic constructs may comprise regulatory elements for gene expression of the coding sequences of the nucleic acid. The regulatory elements may be a promoter, an enhancer, an initiation codon, a stop codon, or a polyadenylation signal.

In certain embodiments, the genetic construct is a vector. The vector can be an Adeno-associated virus (AAV) vector, which encodes at least one Cas9 molecule and at least one gRNA molecule; the vector is capable of expressing the at least one Cas9 molecule and the at least gRNA molecule, in the cell of a mammal. The vector can be a plasmid. The vectors can be used for in vivo gene therapy. The vector may be recombinant. The vector may comprise heterologous nucleic acid encoding the fusion protein, such as the Cas9-fusion protein or CRISPR/Cas9-based gene editing system. The vector may be a plasmid. The vector may be useful for transfecting cells with nucleic acid encoding the Cas9-fusion protein or CRISPR/Cas9-based gene editing system, which the transformed host cell is cultured and maintained under conditions wherein expression of the Cas9-fusion protein or the CRISPR/Cas9-based gene editing system takes place.

Coding sequences may be optimized for stability and high levels of expression. In some instances, codons are selected to reduce secondary structure formation of the RNA such as that formed due to intramolecular bonding.

The vector may comprise heterologous nucleic acid encoding the CRISPR/Cas9-based gene editing system and may further comprise an initiation codon, which may be upstream of the CRISPR/Cas9-based gene editing system coding sequence, and a stop codon, which may be downstream of the CRISPR/Cas9-based gene editing system coding sequence. The initiation and termination codon may be in frame with the CRISPR/Cas9-based gene editing system coding sequence. The vector may also comprise a promoter that is operably linked to the CRISPR/Cas9-based gene editing system coding sequence. The promoter that is operably linked to the CRISPR/Cas9-based gene editing system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, a U6 promoter, such as the human U6 promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. The promoter may also be a tissue specific promoter, such as a muscle or skin specific promoter, natural or synthetic. Examples of such promoters are described in US Patent Application Publication Nos. US20040175727 and US20040192593, the contents of which are incorporated herein in their entireties. Examples of muscle-specific promoters include a SpcS-12 promoter (described in US Patent Application Publication No. US20040192593, which is incorporated by reference herein in its entirety; Hakim et al. Mol. Ther. Methods Clin. Dev. (2014) 1:14002; and Lai et al. Hum Mol Genet. (2014) 23 (12): 3189-3199), a MHCK7 promoter (described in Salva et al., Mol. Ther. (2007) 15:320-329), a CK8 promoter (described in Park et al. PLOS ONE (2015) 10 (4): e0124914), and a CK8e promoter (described in Muir et al., Mol. Ther. Methods Clin. Dev. (2014) 1:14025). In some embodiments, the expression of the gRNA and/or Cas9 protein is driven by tRNAs.

Each of the polynucleotide sequences encoding the gRNA molecule and/or Cas9 molecule may each be operably linked to a promoter. The promoters that are operably linked to the gRNA molecule and/or Cas9 molecule may be the same promoter. The promoters that are operably linked to the gRNA molecule and/or Cas9 molecule may be different promoters. The promoter may be a constitutive promoter, an inducible promoter, a repressible promoter, or a regulatable promoter. The promoter may be a tissue specific promoter. The tissue specific promoter may be a muscle specific promoter. Examples of muscle-specific promoters may include a MHCK7 promoter, a CK8 promoter, and a Spc512 promoter. The promoter may be a CK8 promoter, a Spc512 promoter, or a MHCK7 promoter, for example.

The vector may also comprise a polyadenylation signal, which may be downstream of the CRISPR/Cas9-based gene editing system. The polyadenylation signal may be a SV40 polyadenylation signal, LTR polyadenylation signal, bovine growth hormone (bGH) polyadenylation signal, human growth hormone (HGH) polyadenylation signal, or human β-globin polyadenylation signal. The SV40 polyadenylation signal may be a polyadenylation signal from a pCEP4 vector (Invitrogen, San Diego, CA).

The vector may also comprise an enhancer upstream of the CRISPR/Cas9-based gene editing system, i.e., the Cas9 protein or Cas9 fusion protein coding sequence or sgRNAs, or the CRISPR/Cas9-based gene editing system. The enhancer may be necessary for DNA expression. The enhancer may be human actin, human myosin, human hemoglobin, human muscle creatine or a viral enhancer such as one from CMV, RSV or EBV. Polynucleotide functional polynucleotide enhancers are described in U.S. Pat. Nos. 5,593,972; 5,962,428; and WO/94/016737, the contents of each are hereby fully incorporated by reference for all purposes. The vector may also comprise a mammalian origin of replication in order to maintain the vector extrachromosomally and produce multiple copies of the vector in a cell. The vector may also comprise a regulatory sequence, which may be well suited for gene expression in a mammalian or human cell into which the vector is administered. The vector may also comprise a reporter gene, such as green fluorescent protein (“GFP”) and/or a selectable marker, such as hygromycin (“Hygro”).

The vector may be expression vectors or systems to produce protein by routine techniques and readily available starting materials including Sambrook et al., Molecular Cloning and Laboratory Manual, Second Ed., Cold Spring Harbor (1989), which is hereby incorporated fully by reference for all purposes. In some embodiments the vector may comprise the nucleic acid sequence encoding the CRISPR/Cas9-based gene editing system, including the nucleic acid sequence encoding the Cas9 protein or Cas9 fusion protein and the nucleic acid sequence encoding the at least one gRNA.

The presently disclosed subject matter provides for compositions comprising the above-described genetic constructs. The pharmaceutical compositions as detailed herein can be formulated according to the mode of administration to be used. In cases where pharmaceutical compositions are injectable pharmaceutical compositions, they are sterile, pyrogen free and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, such as immune-stimulating complexes (ISCOMS), Freumis incomplete adjuvant, LPS analog including monophosptloryl lipid A, muramyl peptides, quinone analogs, vesicles suct1 as squalene and squalene, hyaluronic acid, lipids, liposomes, calcium ions, viral proteins, polyanions, polycations, or nanoparticles, or other known transfection facilitating agents.

The transfection facilitating agent is a polyanion, polycation, including poly-L-glutamate (LGS), or lipid. The transfection facilitating agent is poly-L-glutamate, and more preferably, the poly-L-glutamate is present in the composition for genome editing in skeletal muscle or cardiac muscle at a concentration less than 6 mg/ml. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction witt1 the genetic construct. In some embodiments, the DNA vector encoding the composition may also include a transfection facilitating agent such as lipids, liposomes, including lecithin liposomes or other liposomes known in the art, as a DNA-liposome mixture (see for example International Patent Publication No. WO9324640), calcium ions, viral proteins, polyanions, polycations, or nanoparticles, or other known transfection facilitating agents. Preferably, the transfection facilitating agent is a polyanion, polycation, including poly-L-glutamate (LGS), or lipid.

Provided further herein is a kit, which may be used to correct a mutated dystrophin gene. The kit comprises at least a gRNA for correcting a mutated dystrophin gene and instructions for using the CRISPR/Cas9-based gene editing system. Also provided herein is a kit, which may be used for genome editing of a dystrophin gene in skeletal muscle or cardiac muscle. The kit may comprise genetic constructs (e.g., vectors) or a composition comprising thereof for genome editing in skeletal muscle or cardiac muscle, as described above, and instructions for using said composition.

Instructions included in kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips, and flash memory), optical media (e.g., CD ROM), and the like. As used herein, the term “instructions” may include the address of an internet site that provides the instructions.

The genetic constructs (e.g., vectors) or a composition comprising thereof for correcting a mutated dystrophin or genome editing of a dystrophin gene in skeletal muscle or cardiac muscle may include a modified AAV vector that includes a gRNA molecule(s) and a Cas9 molecule, as described above, that specifically binds and cleaves a region of the dystrophin gene. The CRISPR/Cas9-based gene editing system, as described above, may be included in the kit to specifically bind and target a particular region in the mutated dystrophin gene. The kit may further include donor DNA, a different gRNA, or a transgene, as described above.

### (5) Delivery and Administration Routes

Provided herein is a method for delivering the presently disclosed genetic construct (e.g., a vector) or a composition thereof to a cell. The delivery of the compositions may be the transfection or electroporation of the composition as a nucleic acid molecule that is expressed in the cell and delivered to the surface of the cell. The nucleic acid molecules may be electroporated using BioRad Gene Pulser Xcell or Amaxa Nucleofector lib devices. Several different buffers may be used, including BioRad electroporation solution, Sigma phosphate-buffered saline product #D8537 (PBS), Invitrogen OptiMEM I (OM), or Amaxa Nucleofector solution V (N.V.). Transfections may include a transfection reagent, such as Lipofectamine 2000.

Upon delivery of the presently disclosed genetic construct or composition to the tissue, and thereupon the vector into the cells of the mammal, the transfected cells will express the gRNA molecule(s) and the Cas9 molecule. The genetic construct or composition may be administered to a mammal to alter gene expression or to re-engineer or alter the genome. For example, the genetic construct or composition may be administered to a mammal to correct the dystrophin gene in a mammal. The mammal may be human, non-human primate, cow, pig, sheep, goat, antelope, bison, water buffalo, bovids, deer, hedgehogs, elephants, llama, alpaca, mice, rats, or chicken, and preferably human, cow, pig, or chicken.

The genetic construct (e.g., a vector) encoding the gRNA molecule(s) and the Cas9 molecule can be delivered to the mammal by DNA injection (also referred to as DNA vaccination) with and without in vivo electroporation, liposome-mediated, nanoparticle-facilitated, and/or recombinant vectors. The recombinant vector can be delivered by any viral mode. The viral mode can be recombinant lentivirus, recombinant adenovirus, and/or recombinant adeno-associated virus.

A presently disclosed genetic construct (e.g., a vector) or a composition comprising thereof can be introduced into a cell to genetically correct a dystrophin gene (e.g., human dystrophin gene). In certain embodiments, a presently disclosed genetic construct (e.g., a vector) or a composition comprising thereof is introduced into a myoblast cell from a DMD patient. In certain embodiments, the genetic construct (e.g., a vector) or a composition comprising thereof is introduced into a fibroblast cell from a DMD patient, and the genetically corrected fibroblast cell can be treated with MyoD to induce differentiation into myoblasts, which can be implanted into subjects, such as the damaged muscles of a subject to verify that the corrected dystrophin protein is functional and/or to treat the subject. The modified cells can also be stem cells, such as induced pluripotent stem cells, bone marrow-derived progenitors, skeletal muscle progenitors, human skeletal myoblasts from DMD patients, CD133+ cells, mesoangioblasts, and MyoD- or Pax7-transduced cells, or other myogenic progenitor cells. For example, the CRISPR/Cas9-based gene editing system may cause neuronal or myogenic differentiation of an induced pluripotent stem cell.

The presently disclosed genetic constructs (e.g., vectors) or a composition comprising thereof may be administered to a subject by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal intrathecal, and intraarticular or combinations thereof. In certain embodiments, the presently disclosed genetic construct (e.g., a vector) or a composition is administered to a subject (e.g., a subject suffering from DMD) intramuscularly, intravenously or a combination thereof. For veterinary use, the presently disclosed genetic constructs (e.g., vectors) or compositions may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, (e.g., microprojectile bombardment gene guns), or other physical methods such as electroporation (“EP”), “hydrodynamic method,” or ultrasound.

The presently disclosed genetic constructs (e.g., a vector) or a composition may be delivered to the mammal by several technologies including DNA injection (also referred to as DNA vaccination) with and without in vivo electroporation, liposome-mediated, nanoparticle-facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior (TA) muscle or tail.

In some embodiments, the presently disclosed genetic construct (e.g., a vector) or a composition thereof is administered by 1) tail vein injections (systemic) into adult mice; 2) intramuscular injections, for example, local injection into a muscle, such as the TA or gastrocnemius in adult mice; 3) intraperitoneal injections into P2 mice; or 4) facial vein injection (systemic) into P2 mice.

Any of these delivery methods and/or routes of administration can be utilized with a myriad of cell types. Cell types may include, but are not limited to, immortalized myoblast cells, such as wild-type and DMD patient derived lines (e.g., 6.48-50 DMD, DMD 6594 (del48-50), DMD 8036 (del48-50), C25C14 and DMD-7796 cell lines), primal DMD dermal fibroblasts, induced pluripotent stem cells, bone marrow-derived progenitors, skeletal muscle progenitors, human skeletal myoblasts from DMD patients, CD133+ cells, mesoangioblasts, cardiomyocytes, hepatocytes, chondrocytes, mesenchymal progenitor cells, hematopoietic stem cells, smooth muscle cells, and MyoD- or Pax7-transduced cells, or other myogenic progenitor cells. Immortalization of human myogenic cells can be used for clonal derivation of genetically corrected myogenic cells. Cells can be modified ex vivo to isolate and expand clonal populations of immortalized DMD myoblasts that include a genetically corrected dystrophin gene and are free of other nuclease-introduced mutations in protein coding regions of the genome. Alternatively, transient in vivo delivery of CRISPR/Cas9-based systems by non-viral or non-integrating viral gene transfer, or by direct delivery of purified proteins and gRNAs containing cell-penetrating motifs may enable highly specific correction in situ with minimal or no risk of exogenous DNA integration.

Suitable modifications and adaptations of the compositions and methods of the present disclosure, as described herein, are readily apparent, appreciable, and applicable to one of skill in the art. Such modifications and/or adaptations may be made using suitable equivalents without departing from the scope of the present disclosure or the aspects and embodiments disclosed herein.

Having now described the present disclosure in detail, the same will be more clearly understood by reference to the following examples, which are merely intended to illustrate some aspects and embodiments of the disclosure without limiting, in any way, the scope of the disclosure. The disclosures of all journal references, U.S. patents, and publications referred to herein are hereby incorporated by reference in their entireties for all purposes. The present disclosure details multiple embodiments and aspects, illustrated by the following non-limiting examples.

### a. Example 1: Designing CRISPR gRNAs Targeting DMD) Gene Introns

Given that a plurality of DMD patients may be treated with therapy resulting in the excision of exons 45 through 55 of the DMD gene, targeting the introns immediately adjacent to this region (intron 44 before and intron 55 after) became a clear experimental strategy. The first steps were to 1) generate gRNAs that target the adjacent introns and 2) screen gRNA pairs for in vitro efficiency in deleting the genomic region of interest.

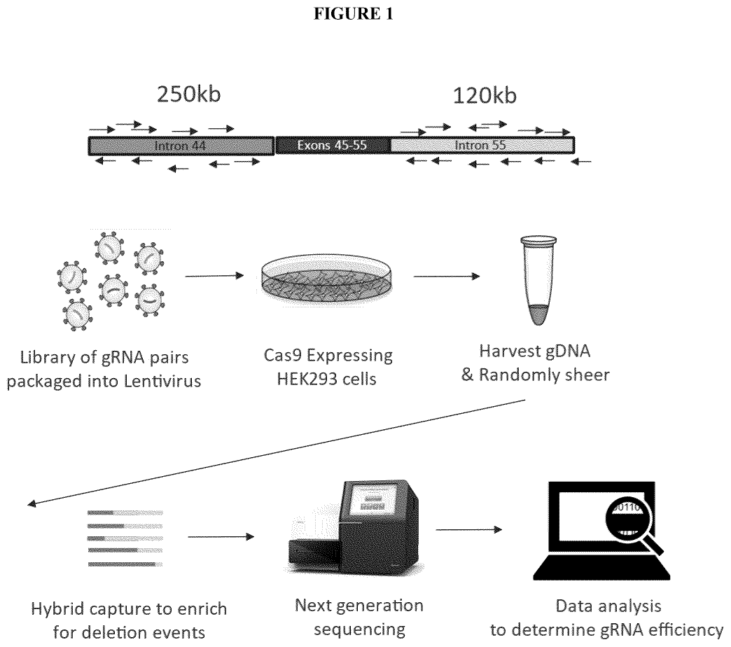

All generated gRNAs are designed to be compatible with Cas9 derived from Staphylococcus aureus (SaCas9) and target either intron 44 or intron 55 of the DMD gene as determined by computational predictions. In total, up to 3.6 million potential gRNA pairs were identified that may potentially induce a deletion of the DMD gene mutational hotspot. Priority was granted to those gRNA pairs computationally predicted to have high specificity, cross-species reactivity to non-human primate (NHP) genomes, and the desired chromosomal position (bias towards producing larger genomic deletions to reach a larger patient population) were selected for inclusion in the high-throughput screen. The prioritized list of approximately 16,000 gRNA pairs were then empirically screened through a combination of high-throughput pooled screens to identify the top editors capable of producing a mutational hotspot deletion. The workflow for gRNA pair generation, screening and selection is schematized in FIG. 1.

### b. Example 2: Screening of CRISPR gRNA Pairs Targeting DMD Gene Mutational Hotspot

Having designed and prioritized the generated gRNA pairs targeting the genomic region of interest, the next step was to experimentally screen and determine the biological activity of these sgRNA pairs.

A library of 15634 guide RNA pairs were cloned into a lentivirus backbone vector. Briefly, 293 FT cells were transfected in cis with the plasmid pool of interest (i.e., encoding the gRNA pairs) and lentivirus packaging plasmids (psPax2/pmD2.G). Lentivirus was harvested, concentrated, frozen at −80° C., and titered in 293 FT cells via measurement of a fluorescent reporter's expression by flow cytometry. NGS sequencing using an Illumina MiSeq platform was performed to confirm representation of each guide RNA in the resulting lentiviral library.

293 FT cells expressing SaCas9 were transduced with the lentivirus library at an MOI of 0.2 to ensure that on average each cell had one viral integrant. Briefly, cells were seeded into Lentivirus using polybrene, and media was changed 24 hours post transduction. Cells were selected with puromycin at three days post-transduction and harvested at 20 days post transduction. Genomic DNA was extracted, and gRNA expression cassettes were amplified via PCR prior to NGS sequencing on an Illumina MiSeq platform to assess representation of each guide RNA in the transduced cells. In a separate library preparation, gDNA was sheared, and hybrid capture was performed using custom probes to enrich for gDNA containing expected deletion events. NGS libraries of the genomic deletions were prepared using custom full-length Y-adapters and sequenced on a Nova-seq platform. A novel analysis pipeline was used to identify the gRNAs responsible for each observed deletion event.

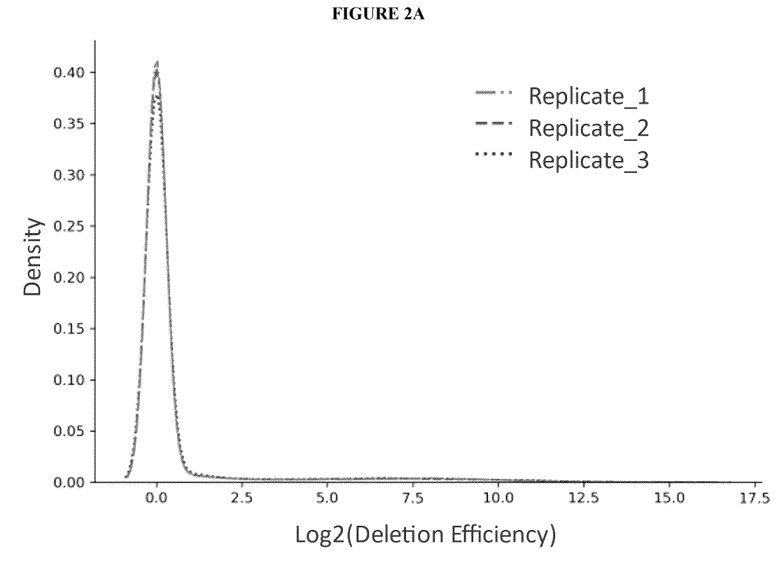

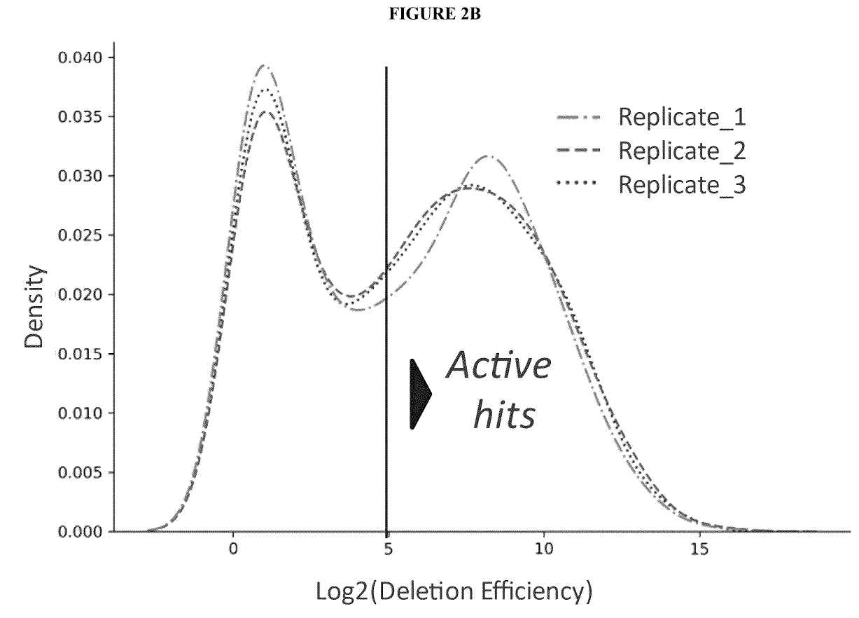

The deletion efficiency of each gRNA pair in the screen was calculated and the distribution of editing rates plotted (FIG. 2A). As expected, most gRNA pairs did not produce a genomic deletion. Additional filtering of the data to only plot gRNA pairs where at least one deletion event was observed, allowed for narrowing the ˜16,000 pairs screened to the top 138 pairs (FIG. 2B).

These 138 most efficient gRNA pairs were further analyzed in an arrayed screen format. Each guide RNA pair was cloned into a lentivirus backbone plasmid. Lentivirus production in 293 FT cells, harvest, and concentration was performed as previously described. Healthy SaCas9 expressing immortalized myoblasts were transduced at a uniform level of lentivirus transduction (>90% of cells transduced based upon titer) with each guide RNA in triplicate at the time of seeding. Briefly, myoblasts were seeded cells into Lentivirus using polybrene, and media was changed 24 hours post transduction. The myoblasts were differentiated at 48 hours post-transduction until the timepoint of harvest (7 days). Total cellular RNA was extracted, and cDNA were generated by reverse transcription. Custom assays were designed to detect both edited and wild type DMD transcripts via digital droplet PCR (ddPCR) using the Bio-Rad QXOne platform. The proportion of edited transcripts compared to total transcripts was quantified, followed by additional statistical analyses, to determine the deletion efficiency of each gRNA pair in the pooled screen.

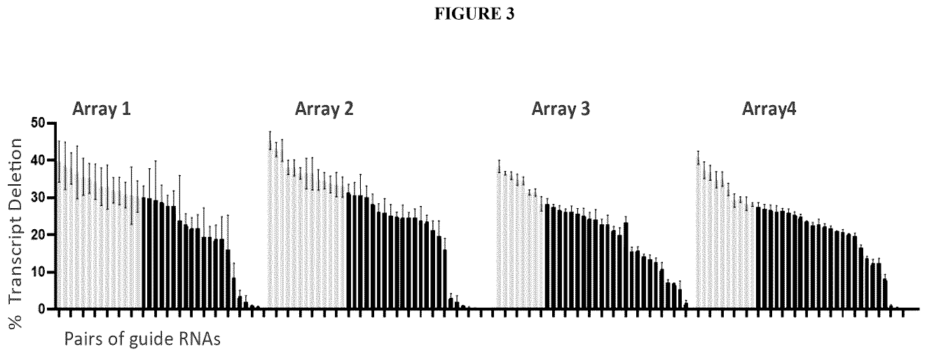

These results demonstrated that 46 of the 138 gRNA pairs tested demonstrated editing efficiency in excess of 30% (FIG. 3; Table 5). Notably, these deletion efficiencies greatly exceeded those of previously reported pairs for CRISPR-mediated hotspot excision (see, WO2016/161380 and WO2020/214609, the latter of which served as benchmark controls in validation experiments). These screens collectively showed that the methods outlined above result in gRNA pairs that induce high rates of editing at the target locus as part of a high-efficiency CRISPR system.

### c. Example 3: Validation of Screening in Healthy and Diseased Cells

Having identified numerous gRNA pairs were able to induce CRISPR-mediated deletions of the mutational hotspot with high efficiency within a screening paradigm, the next step was to confirm these results. To do this, the top-performing quartile from the screens (46 gRNA pairs in total) were tested in either healthy myoblasts or patient-derived diseased myoblasts containing an Exon 52 deletion in the DMD gene-hereafter referred to as A52 cells. Transduction, differentiation, harvest, RNA extraction, cDNA preparation, and ddPCR transcript editing assessment were performed as previously described.

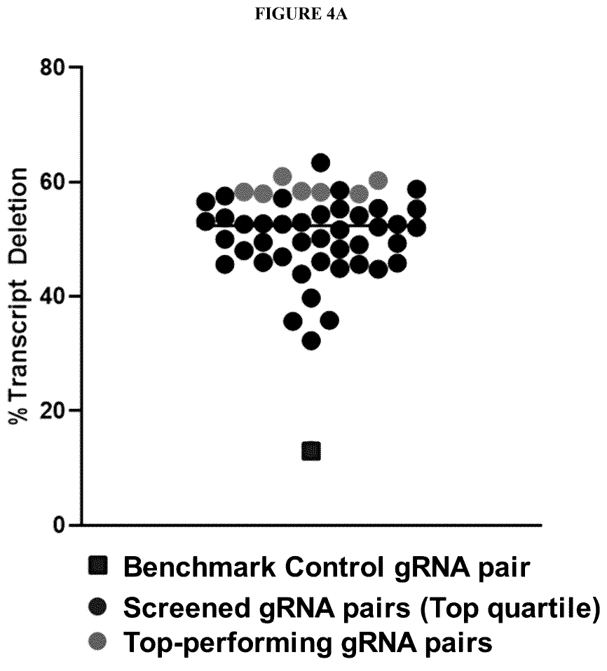

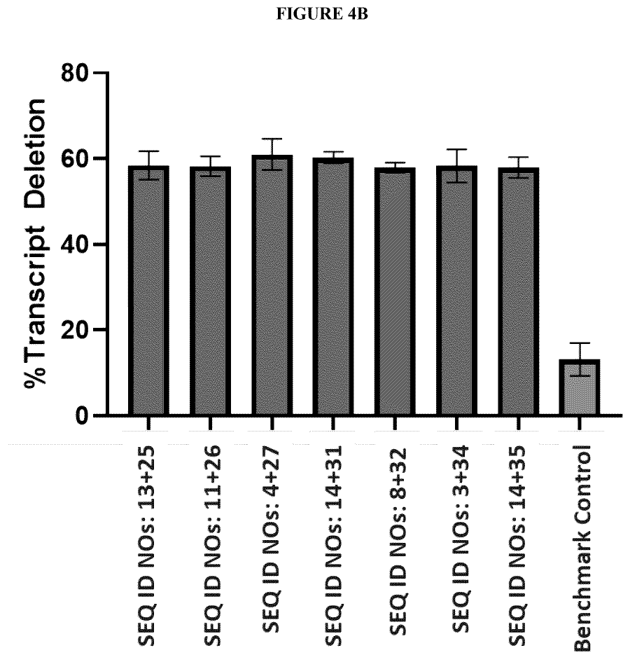

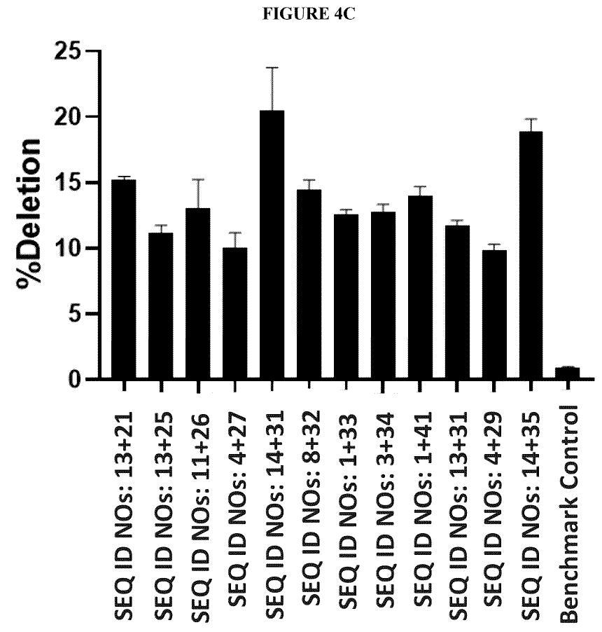

The results demonstrated that each identified gRNA pair exhibited robust editing as compared to benchmark controls in the 452 cells (FIG. 4A; Table 6). Notably, several gRNA pairs in this experiment exhibited editing efficiencies (as measured by cDNA) near 60% in the diseased cells, as compared to less than 20% for the benchmark controls (FIG. 4B). Moreover, this superiority over the benchmark controls persisted in the genomic DNA of healthy cells (FIG. 4C). Taken together, these results confirm the identification of the high efficiency gRNA pairs using the screening methods and demonstrate the comparative superiority over previously reported gRNA pairs.

### d. Example 4: Restoration of Dystrophin Expression in Diseased Primary Cells

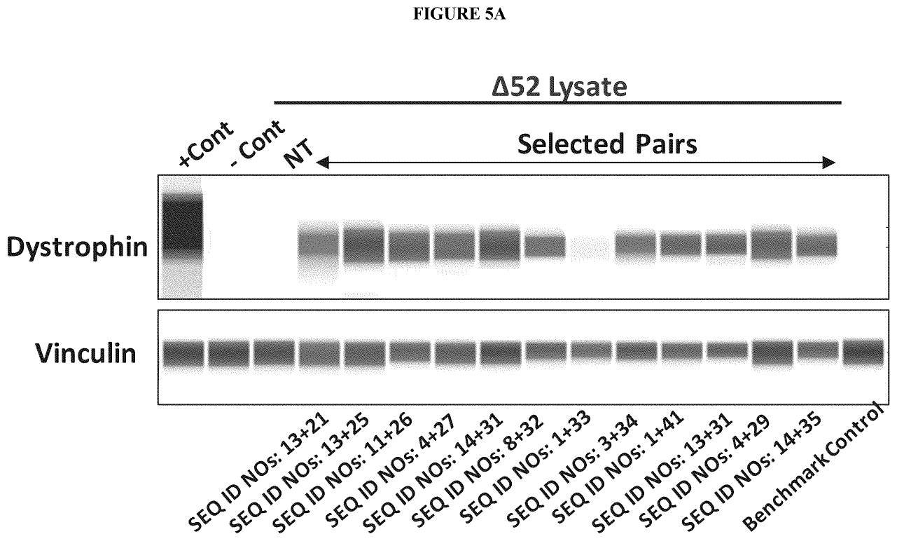

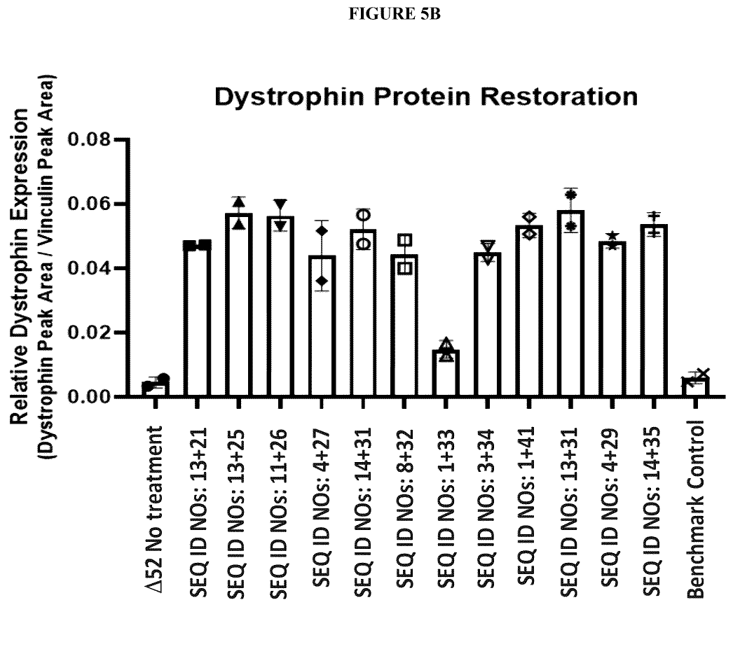

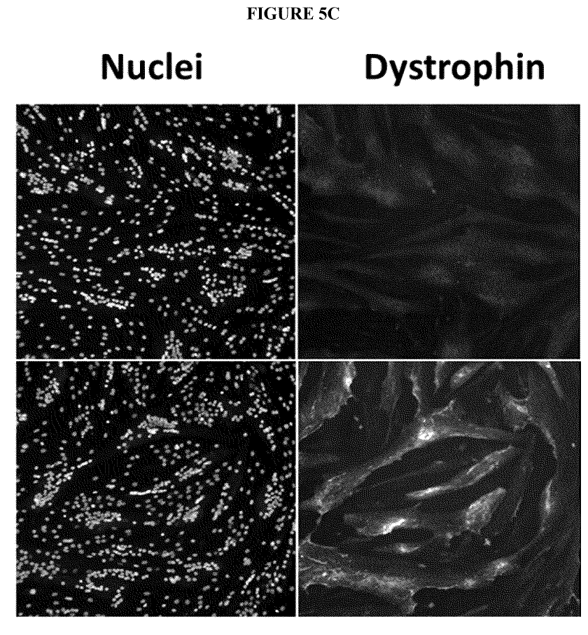

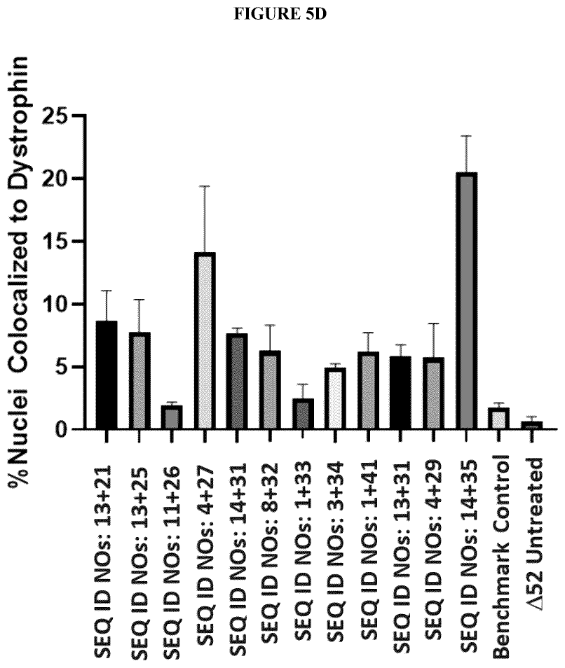

Having validated the results from the gRNA screens and identified numerous high-efficiency gRNA pairs capable of inducing CRISPR-mediated deletions of the dystrophin mutational hotspot, the next step was to confirm the induction of editing by assessing restored expression of a dystrophin protein (less the domains encoded by exons 45-55—hereafter referred to as dystrophin-358 [in reference to the size of the edited protein in kDa]). Based on the validation experiments above, 12 gRNA pairs were selected for further analysis. Protein expression of dystrophin-358 was confirmed either in A52 myoblasts stably expressing SaCas9 by JESS capillary western blot (FIGS. 5A-5B) or in Clone A03 Δ52 cells by IHC analysis with a C-terminal antibody (FIGS. 5C-5D).

These results demonstrated that use of any one of the 12 selected gRNA pairs with a Cas9 CRISPR system restored dystrophin expression in A52 myoblasts better than the benchmark control (FIG. 5A). Quantification of the protein expression, as measured by the replicate lanes, is included in FIG. 5B. Additionally, restoration of detectable dystrophin protein detection in A52 myoblasts was observed following treatment with a CRISPR system and any of the 12 selected gRNA pairs (FIG. 5C-representative images shown). Quantification of replicate wells based on the amount of protein relative to nuclei is included in FIG. 5D. Taken together, these results showed enhanced detection of a dystrophin protein following treatment with the selected gRNA pairs in a CRISPR/Cas9 system. These results suggested that the dystrophin deficiency caused by a truncation mutation within the genomic region of interest can be treated within a cell with any one of the tested gRNA pairs, thereby supporting the use of these gRNA pairs as a therapeutic intervention for DMD and BMD patients.

### e. Example 5: Vector Components for Gene Editing System Targeting the DMD Gene

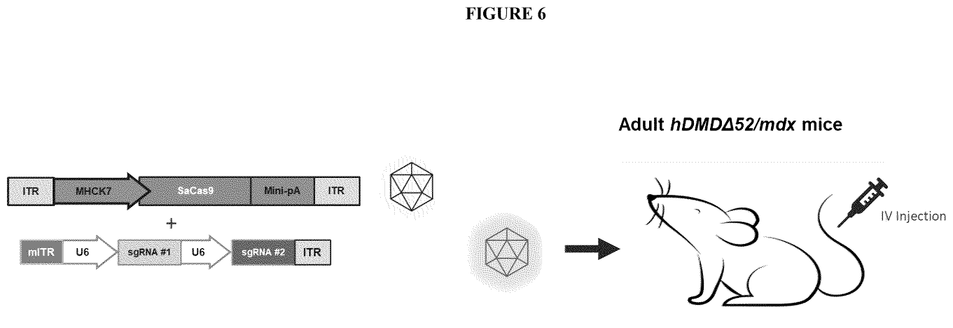

Both dual-vector and single-polynucleotide CRISPR/Cas9 systems were developed for the treatment of DMD. In the case of the dual-vector system (schematized in FIG. 6), the ratio of Cas9-containing vector to gRNA pair containing vector is crucial to successful treatment. Mouse experiments using the dual-vector system may use ratios of 1:1, 1:2, 1:3, 1:4, 1:5, 1:6, 1:7, 1:8, and/or 1:9, as needed to drive the highest deletion efficiency.

Among other advantages to a single-polynucleotide system, one primary benefit over a dual-vector system is having all necessary editing components on a single vector, thereby streamlining vector production manufacture. Additionally, only a single therapeutic agent (i.e., a viral vector) must transduce a cell for therapy, as opposed to requiring two distinct transduction events. Furthermore, a single-polynucleotide system negates the need to consider vector ratios. Sequences included in some or all of the herein described dual-vector and single-polynucleotide approaches are shown in Table 6.

### f. Example 6: Examining Effect of Gene Orientation on Gene Editing

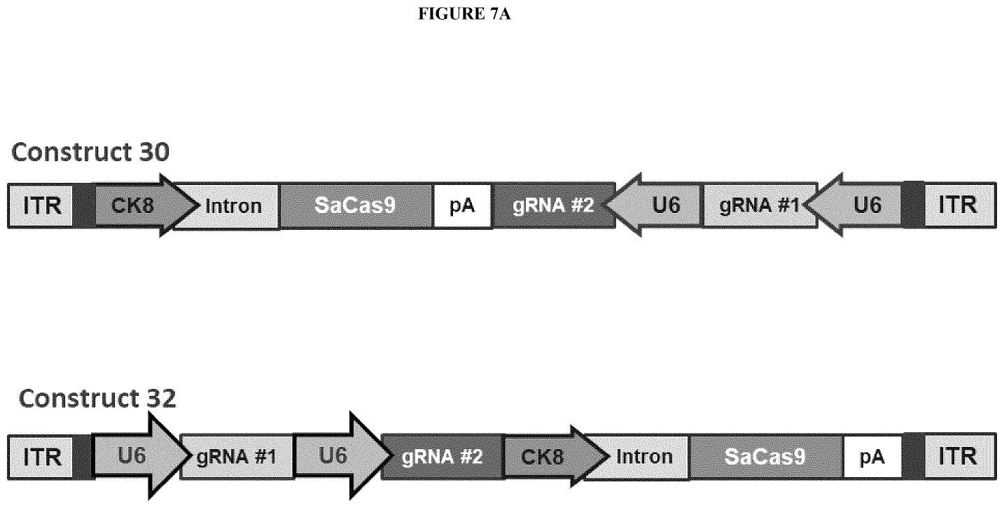

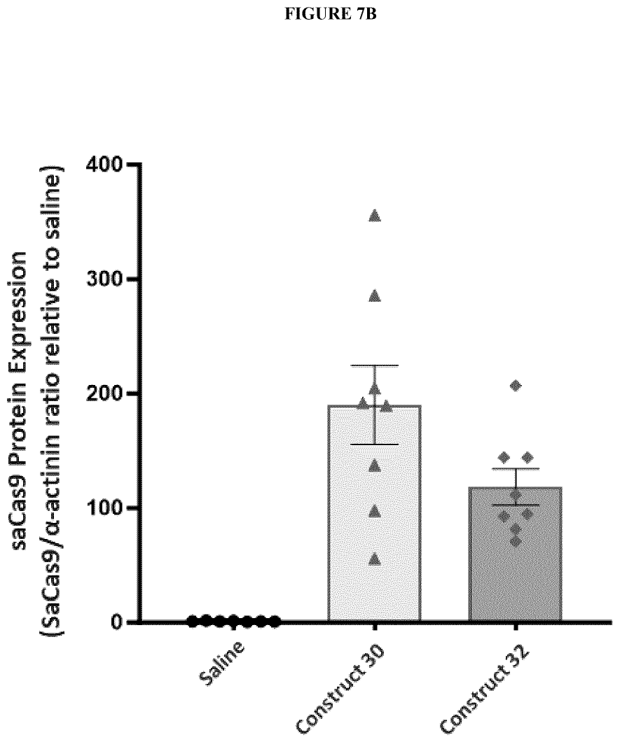

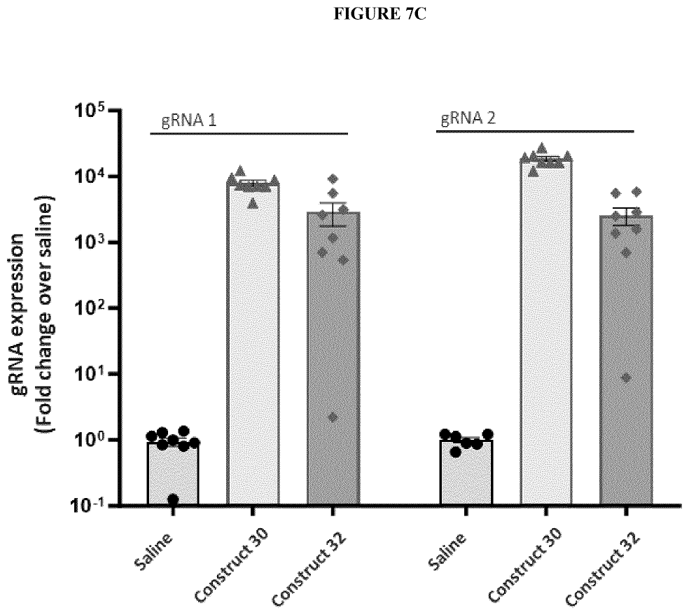

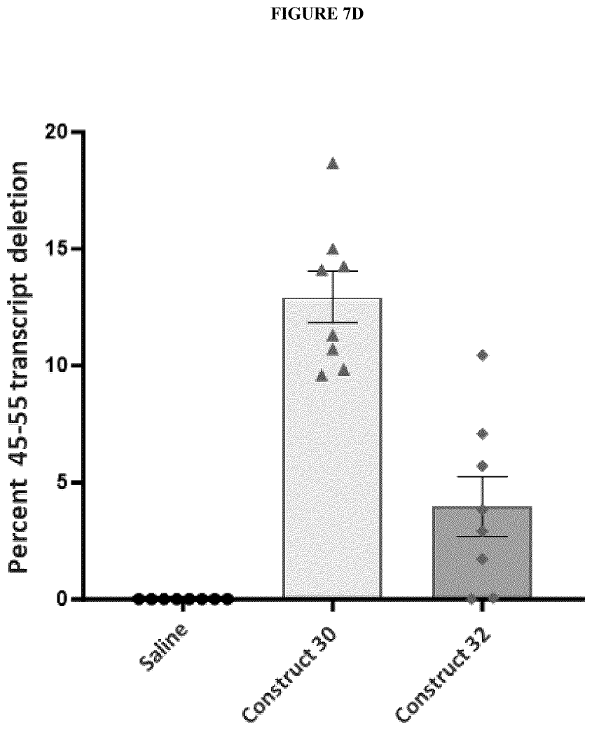

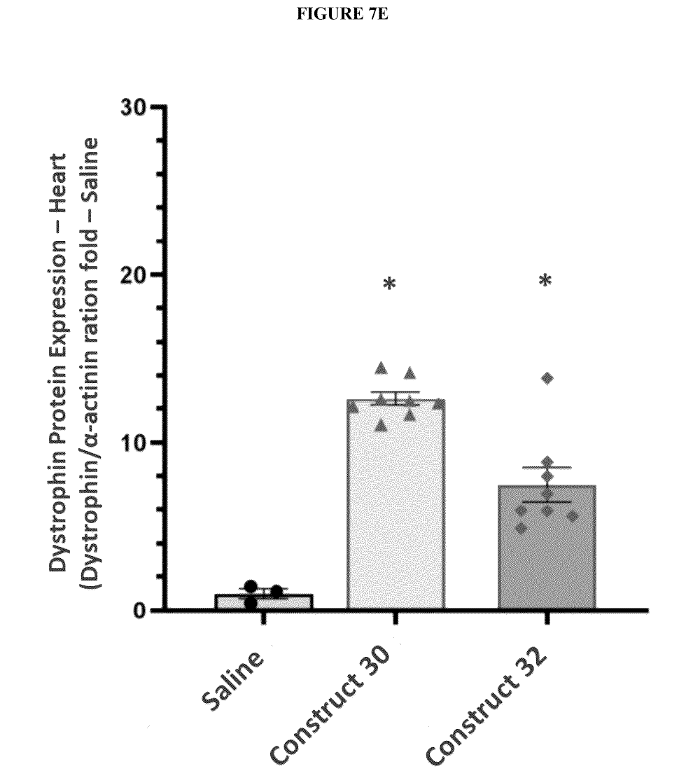

Because of the notable advantages to single-polynucleotide delivery over the dual-vector approach, additional studies were designed to maximize gene editing efficiency using this approach. Particularly, two genetic constructs were designed and synthesized for testing of in vivo gene editing in adult hDMDΔ52/mdx mice (schematized in FIG. 7A). Briefly, AAV.rh74 was administered systemically (IV) to hDMDΔ52/mdx mice at a dose of 2.5E14 vg/kg, DMD gene editing and dystrophin protein expression were assessed after 12 weeks.

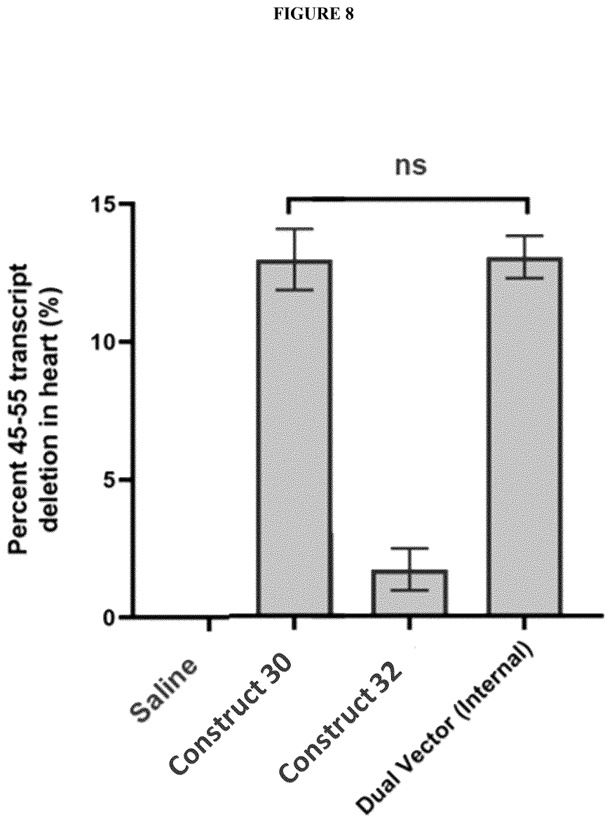

Construct 30, featuring Cas9 and gRNA expression being driven in opposition to one another rather than in the same direction (i.e., the historic conformation), exhibited more robust Cas9 protein (FIG. 7B) and gRNA (FIG. 7C) expression in the cardiac muscle as compared to Construct 32, which features the historic conformation. Furthermore, Construct 30 also exhibited approximately five-fold greater mutational hotspot deletion, as measured by ddPCR for the edited transcript in heart muscle tissue (FIG. 7D). Additionally, use of Construct 30 resulted in greater amounts of dystrophin protein in the heart muscle following gene editing with the same benchmark control gRNAs (FIG. 7E). In addition to outperforming Construct 32, Construct 30 also induced an equivalent amount of gene editing as a dual-vector system using the same benchmark control gRNAs (FIG. 8).

Taken together, these experiments demonstrated that orientation of components within a genetic construct greatly influenced gene editing, despite having the same features and sequences. Particularly, the orientation of Construct 30 boosted Cas9 and gRNA expression and resulted in higher editing efficiency and dystrophin protein restoration in vivo.

### g. Example 7: Optimization of Genetic Constructs for Gene Editing Via Plasmid Transfection

Having observed that a single-polynucleotide approach could work at least as well as the dual-vector approach by adjusting orientation of the expressible components, further optimization of the genetic constructs was then undertaken to further enhance gene editing efficiency by increasing packaging efficiency in AAV vectors and/or expression of encoded components within the construct.

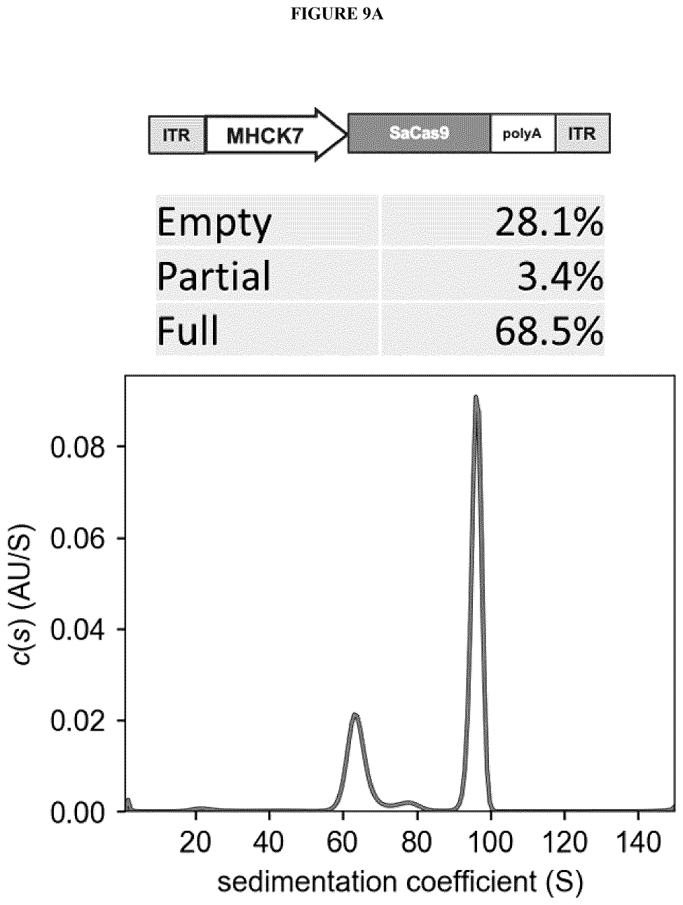

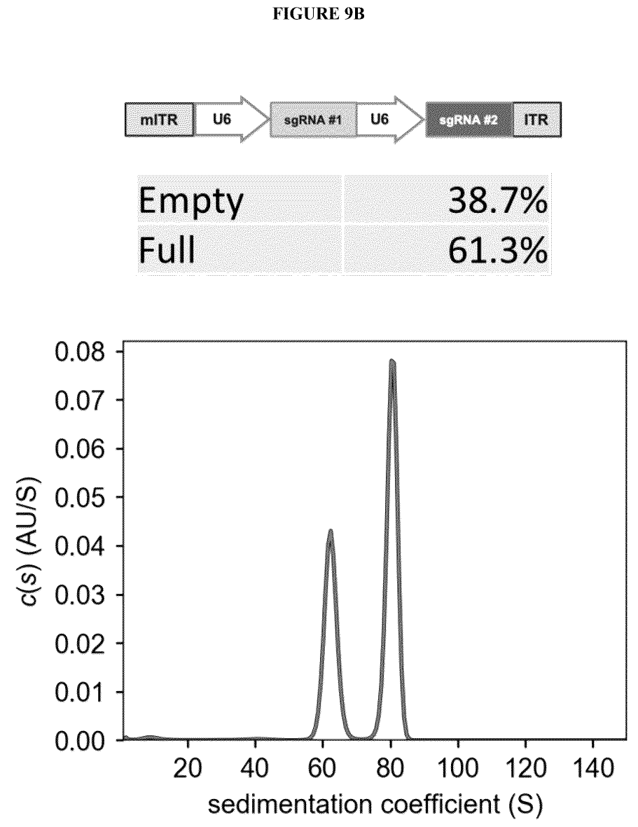

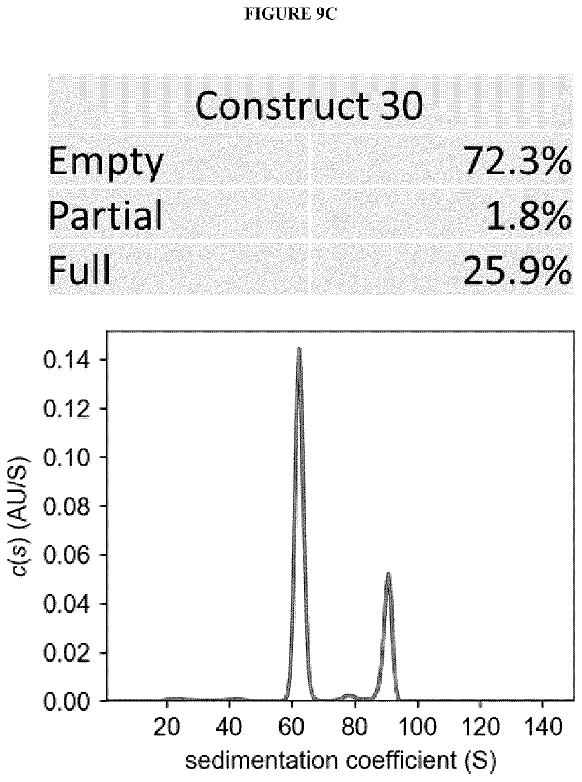

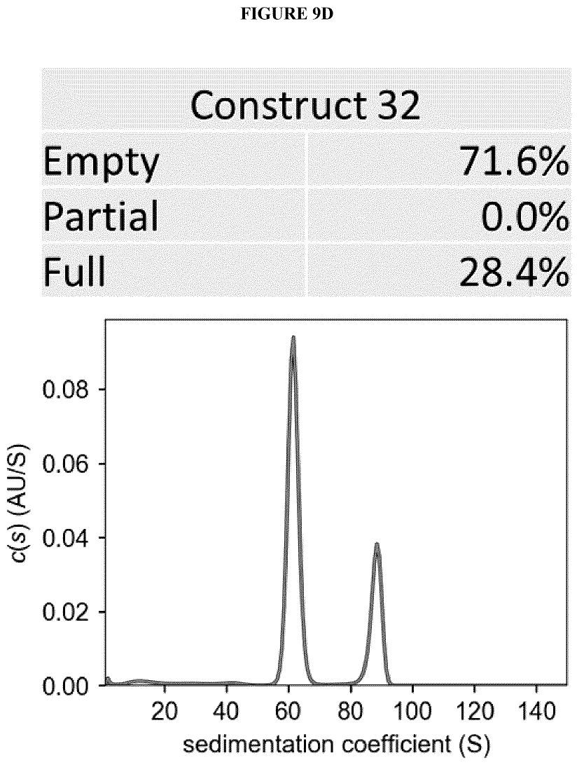

First, it was noted that compared to the dual-vector system, the single-polynucleotide system was less efficient in packaging into AAVs, likely due to AAV genome size constraints. Indeed, while 68.5% of vectors produced contain a Cas9-encoding genetic construct (FIG. 9A) and 61.3% of vectors produced contain a gRNA-encoding genetic construct (FIG. 9B), only 28.4% of Construct 32 and 25.9% of Construct 30 were fully encapsidated into an AAV vector (FIGS. 9C, 9D).

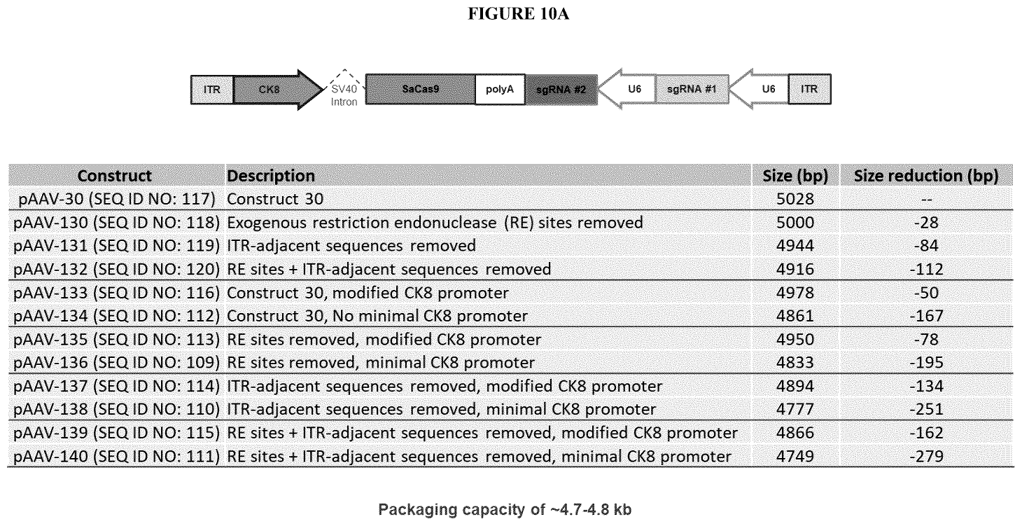

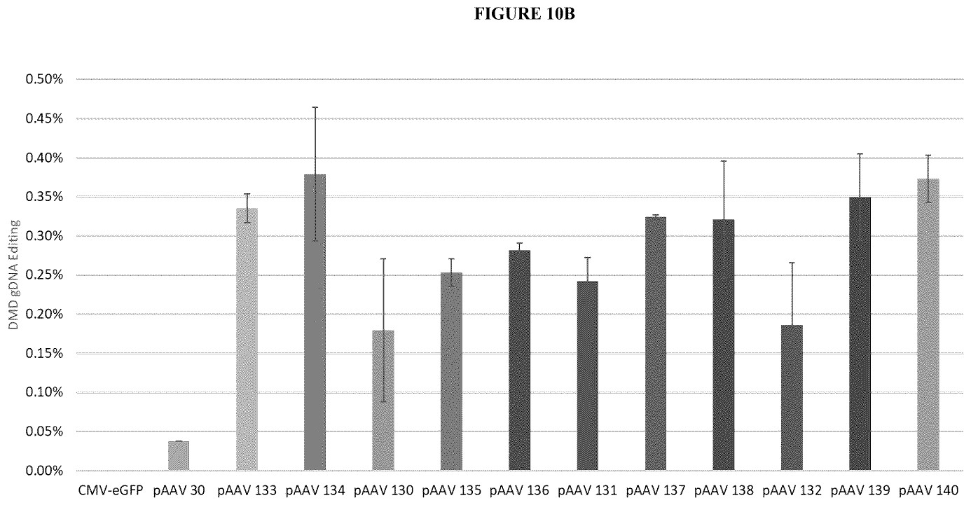

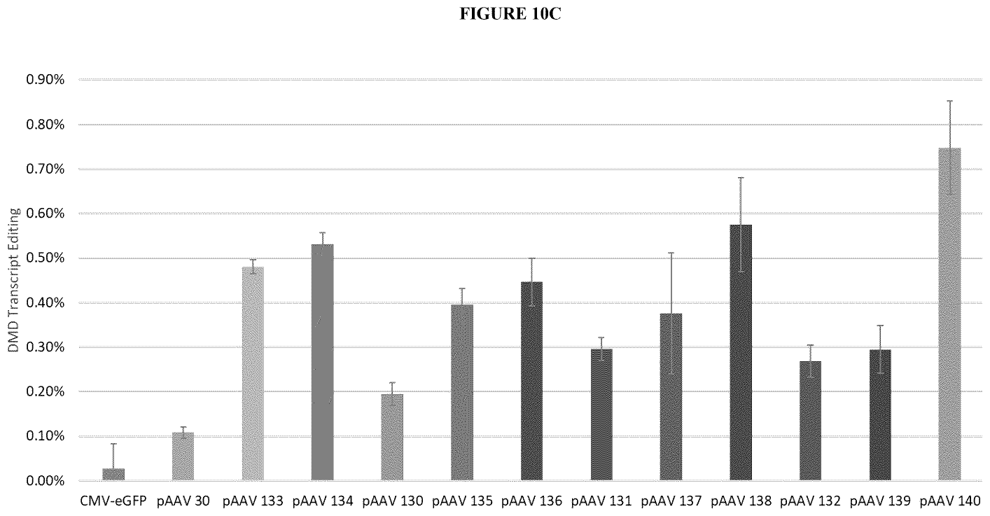

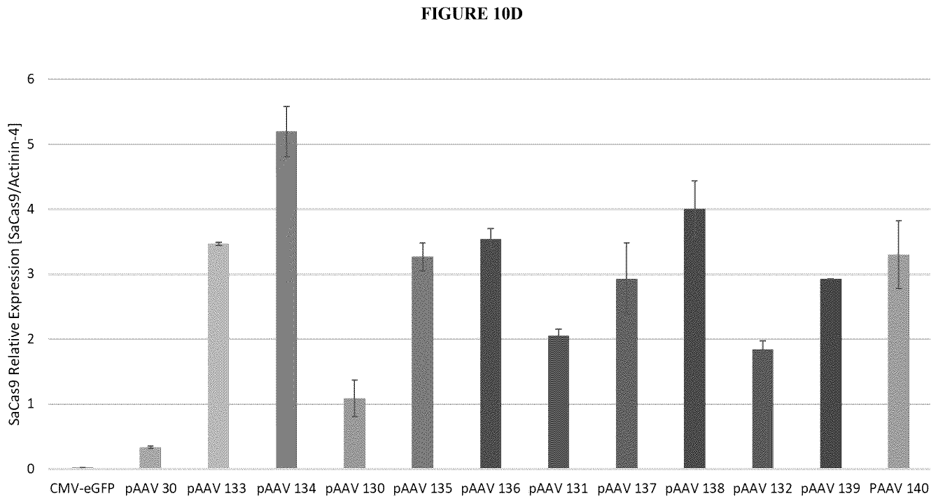

This observation led to the design and production of various genetic constructs with reductions in AAV genome size with the goal of increasing packaging efficiency into AAV vectors (FIG. 10A). In order to isolate the effects of packaging and/or expression of gene editing components, all constructs were generated with the previously characterized benchmark control gRNAs targeting the dystrophin mutational hotspot (see above). These constructs were then transfected into healthy myoblasts. Cells were collected 48 hours post transfection to assess gDNA and transcript editing (FIGS. 10B-10C). All shortened constructs outperformed the original vector, Construct 30, particularly those without the SV40 intron within the CK8 promoter. Additionally, JESS capillary western blot analysis for SaCas9 expression suggested that the increase in observed editing may be linked to greater SaCas9 protein expression, as significant increases were observed from constructs lacking the intronic portion of the CK8 promoter as opposed to those in which it was retained (FIG. 10D).

These results demonstrated that the CK8 promoter without its internal intron was capable of driving up to five times greater SaCas9 expression in healthy myoblasts. Additionally, they showed that removal of internal sequences (including restriction sites, cloning scars, and the sequences flanking the ITR sequences) was not deleterious to DMD gene editing via plasmid transfection.

### h. Example 8: Testing Optimized Genetic Constructs for Gene Editing Via AAV Transductions

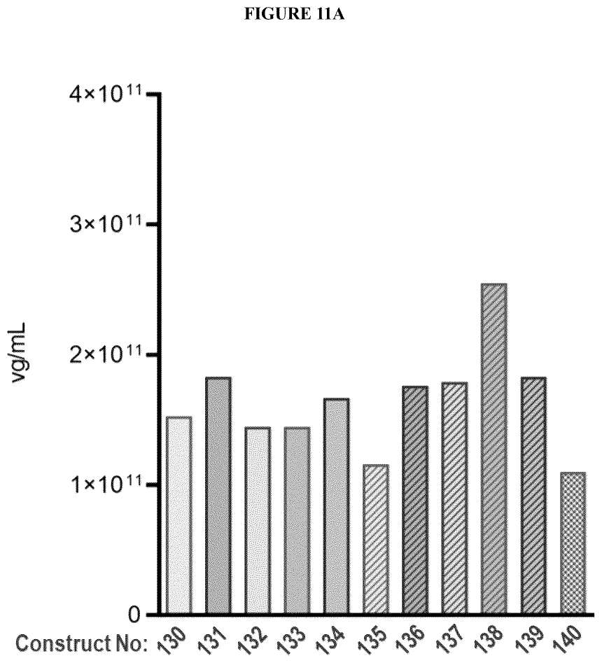

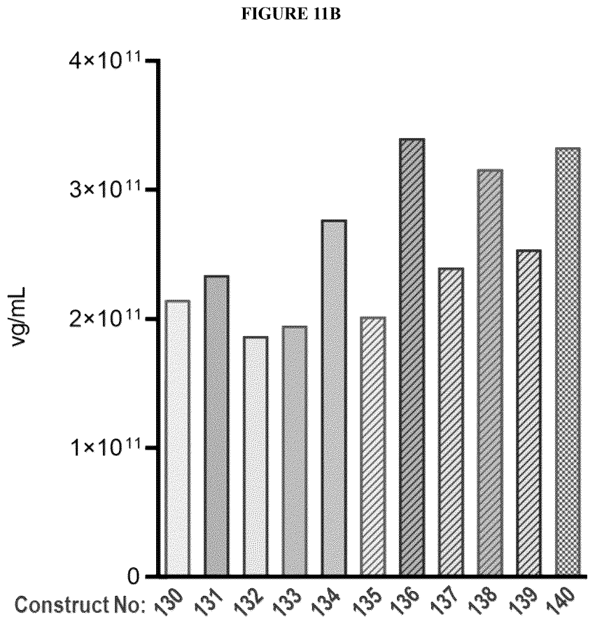

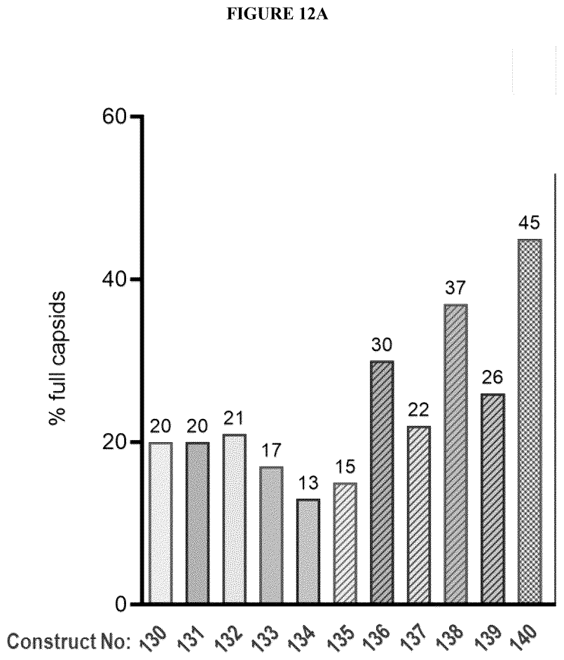

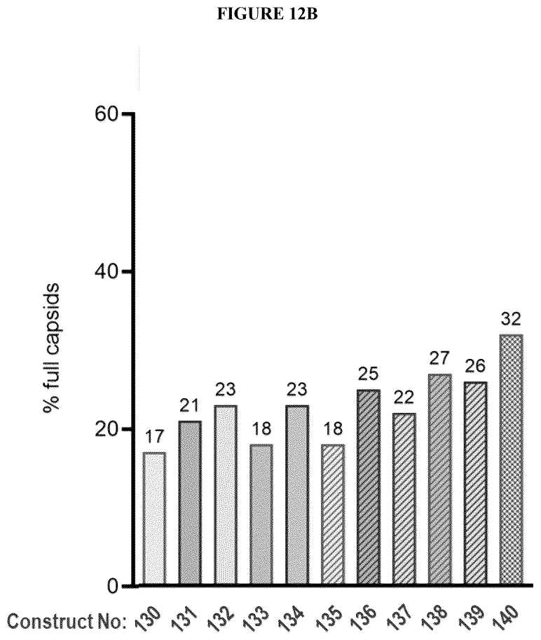

Having observed that the designed genetic constructs showed greater efficacy and capability to express Cas9, the next step was to test their ability to be packaged within AAV vectors. The genetic constructs were used to produce either AAVrh74 or MyoAAV viral vectors. Viral titers showed that each of these constructs were capable of being produced in excess of 1×10¹¹ vg/mL with either viral vector (FIGS. 11A-11B). The resultant vectors were then analyzed by size exclusion chromatography-multi angle light scattering (SEC-MALS) to determine the proportion of capsids containing full AAV genomes. The results showed that the three smallest constructs, pAAV140, pAAV138 and pAAV136, had the largest share of full capsids at 45%, 37% and 30%, respectively, in AAVrh74 vectors, as compared with only 20% for the largest of the designed genetic constructs, pAAV130 (FIG. 12A). Similarly, pAAV140 and pAAV138 had the largest proportion of full MyoAAV capsids at 32% and 27%, respectively, as compared to only 17% for pAAV130 (FIG. 12B).

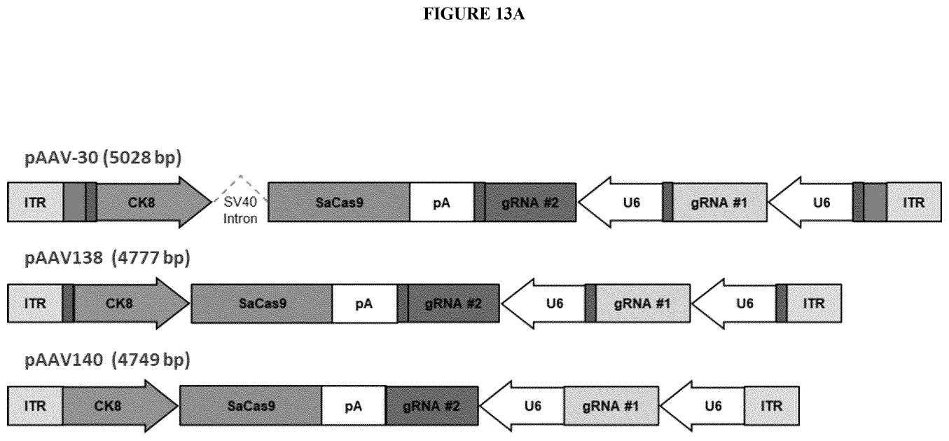

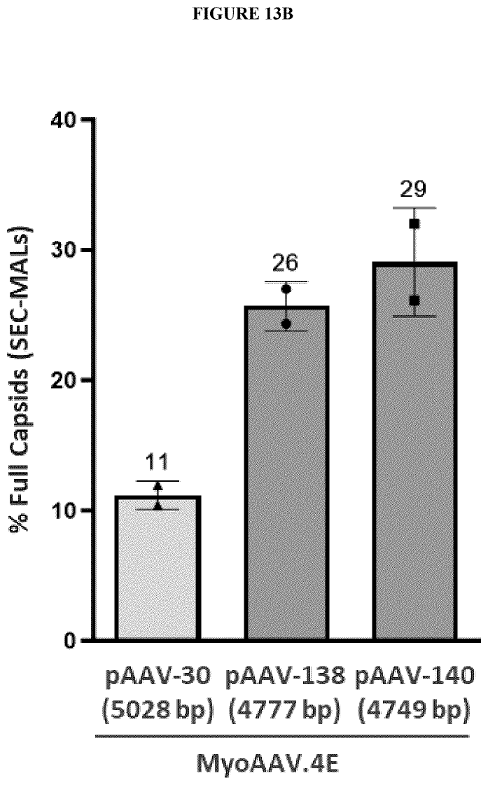

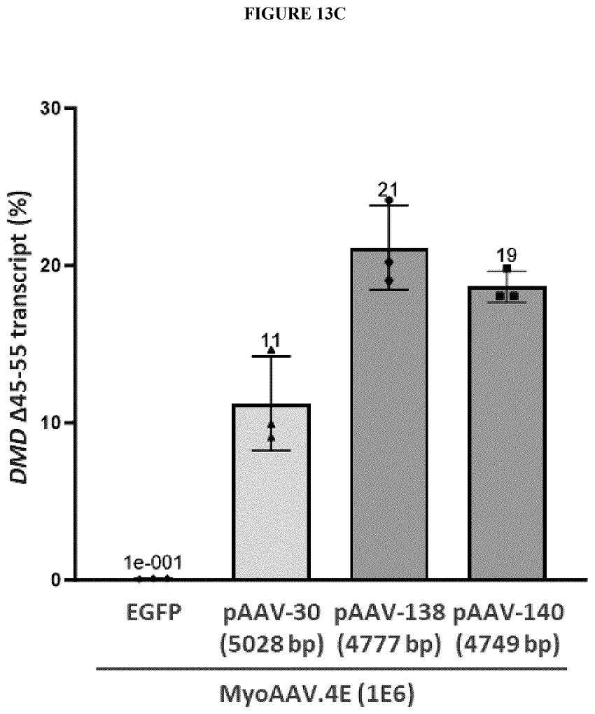

The smallest optimized constructs (138, and 140; schematized in FIG. 13A) were then tested against Construct 30 to assess the size effects on both encapsidation and gene editing efficiency in A03 Δ52 myoblasts. Briefly, select genetic constructs were transduced into myotubes that were differentiated on gelatin-coated plates. 12 days post-transduction, cells were collected and processed for molecular outcomes. DMD transcript editing efficiency was determined by QXOne ddPCR.

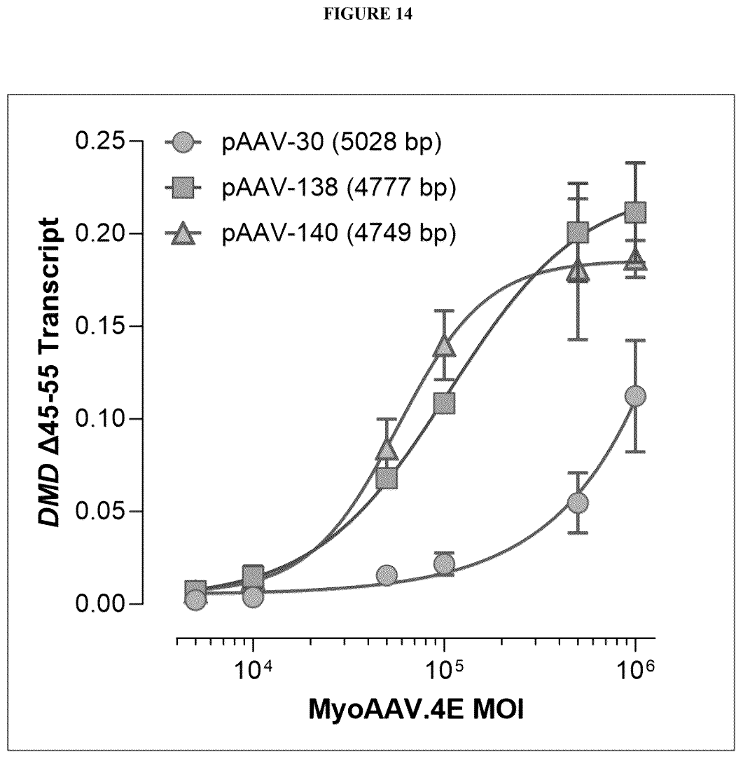

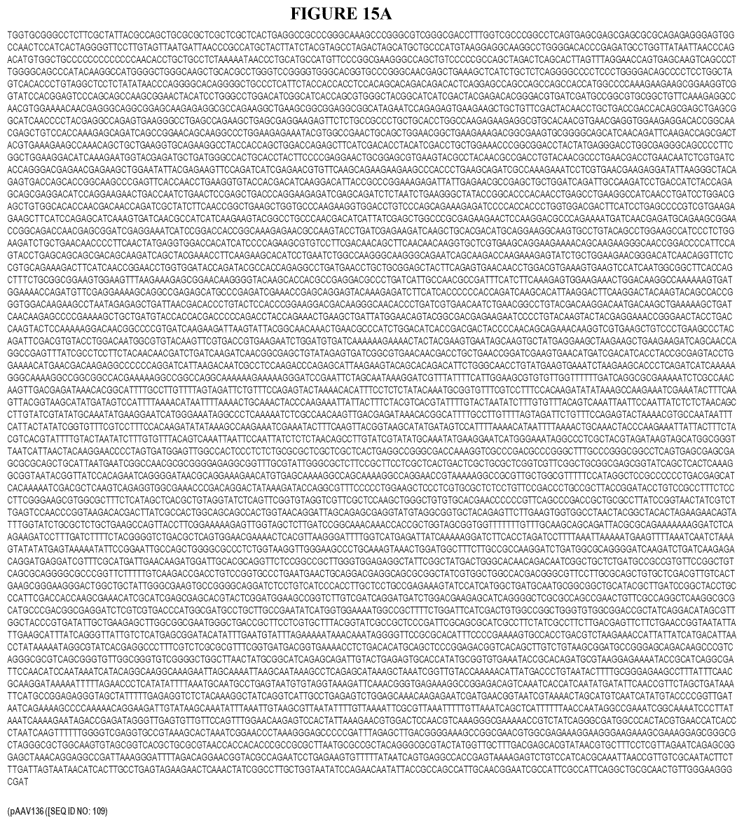

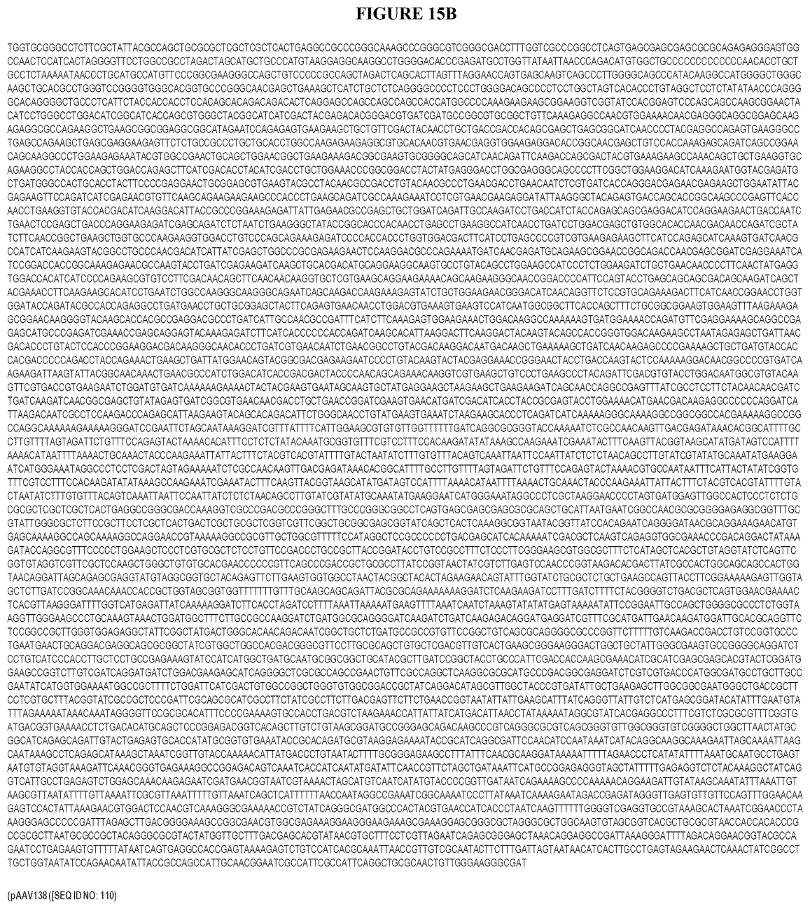

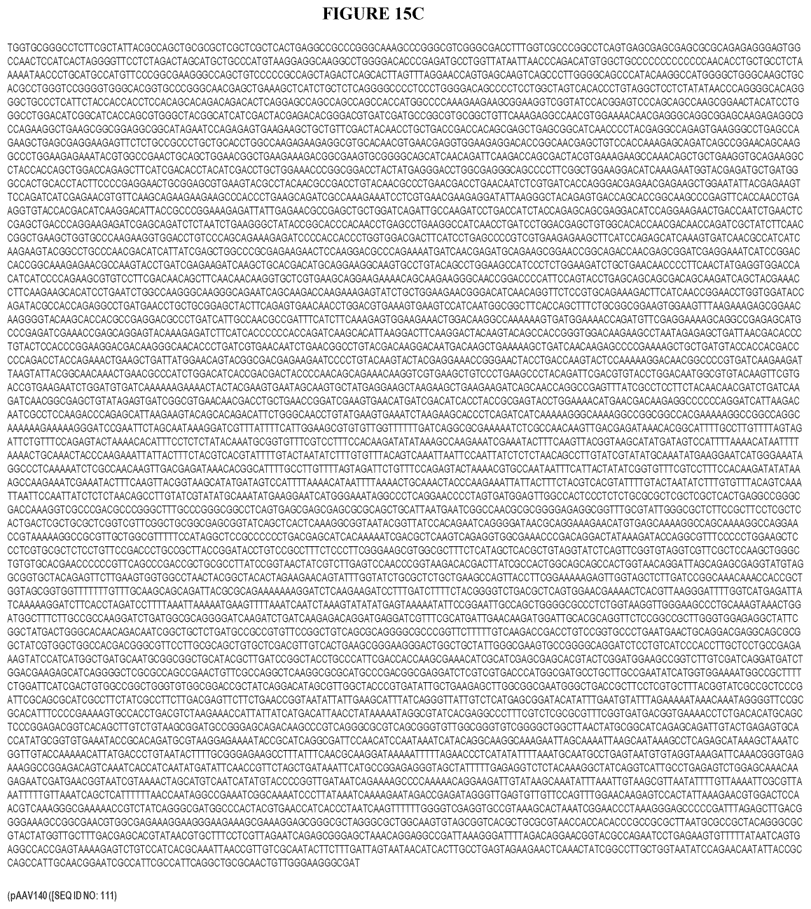

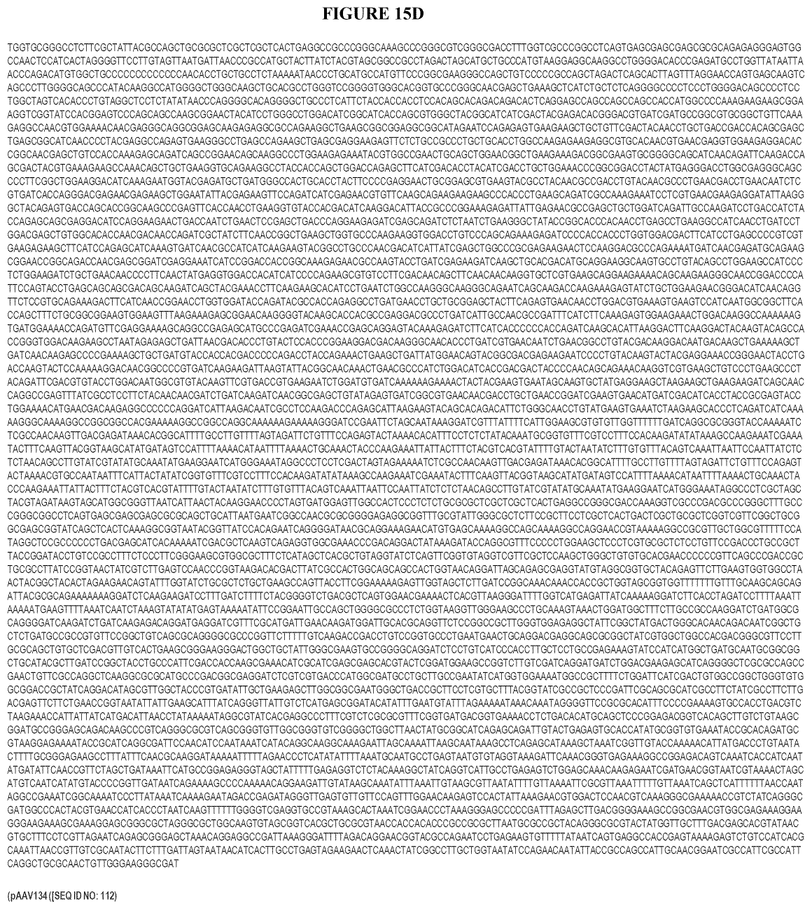

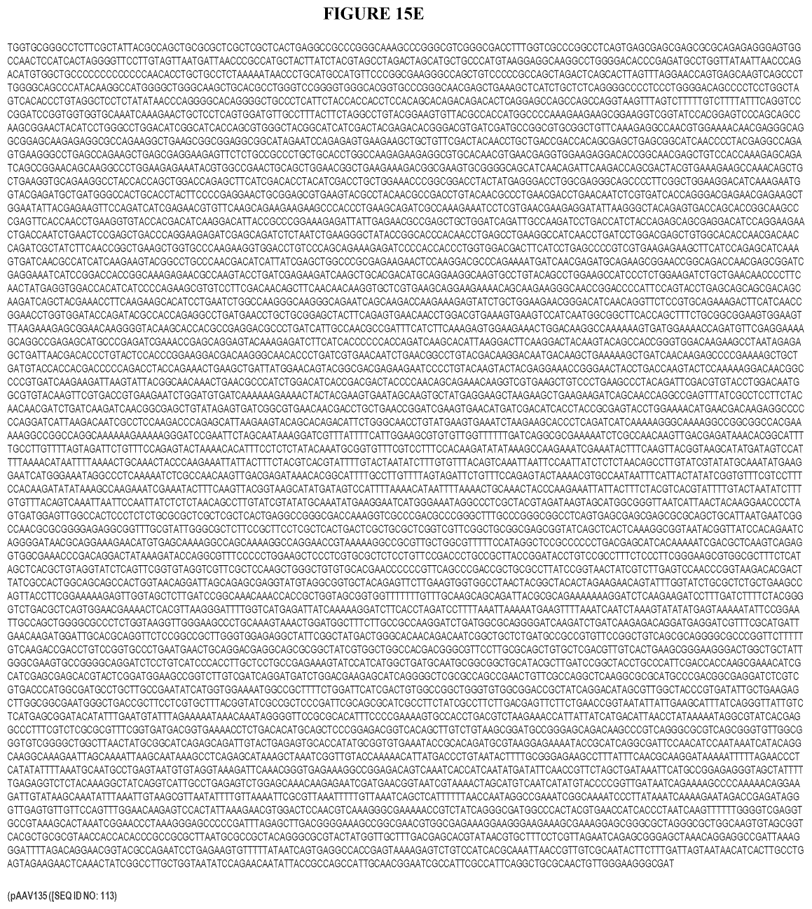

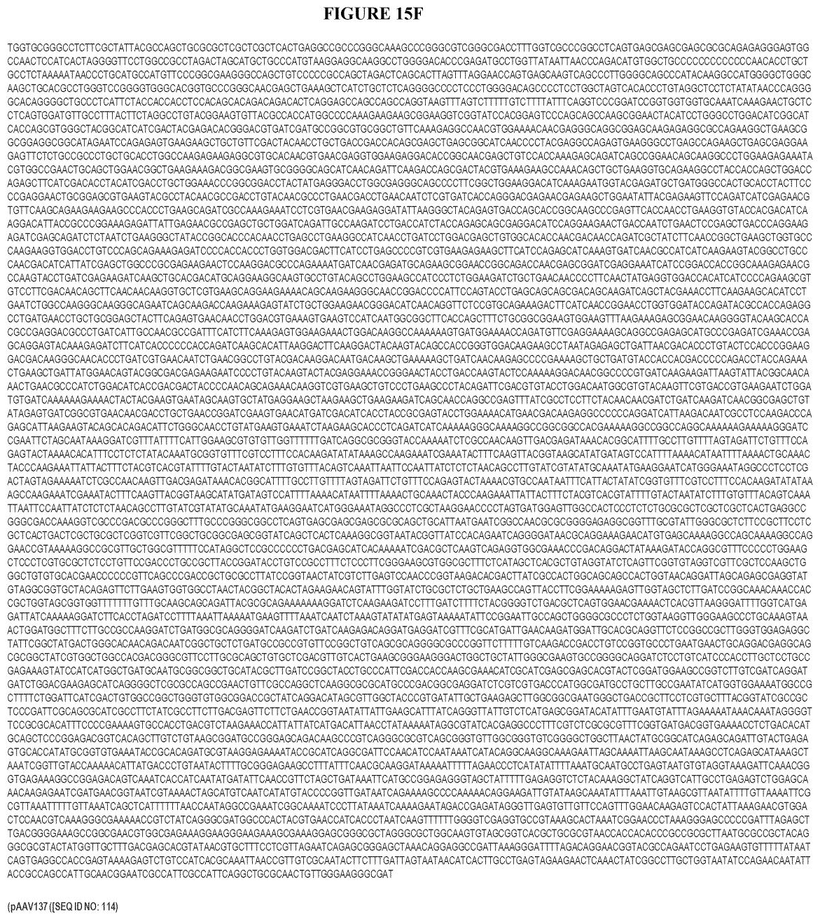

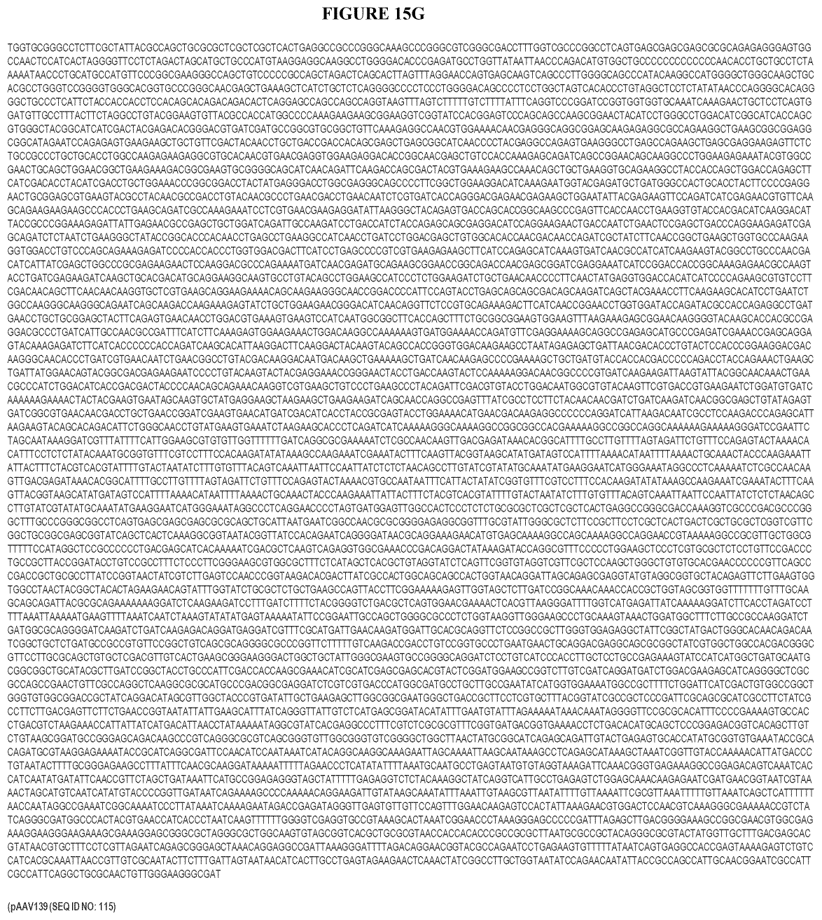

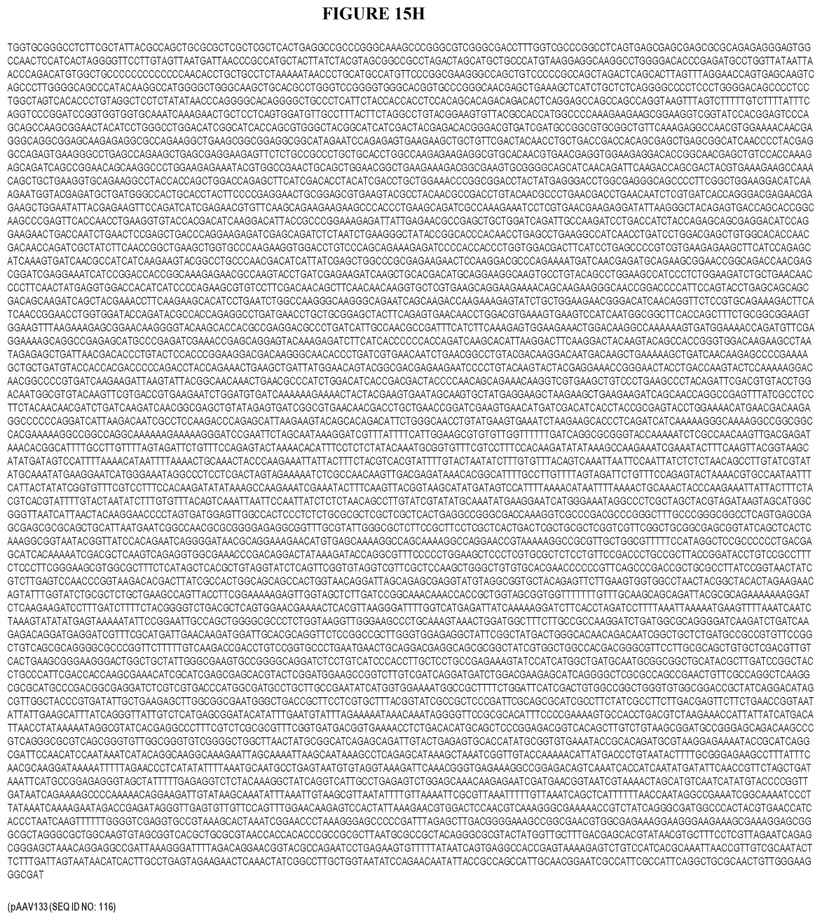

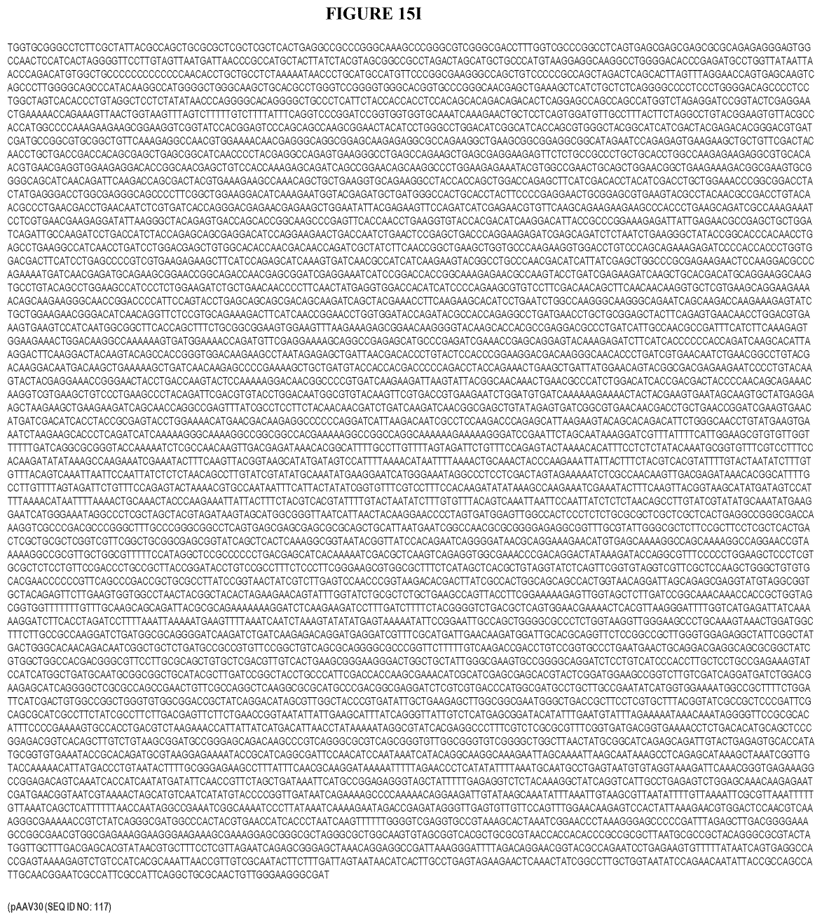

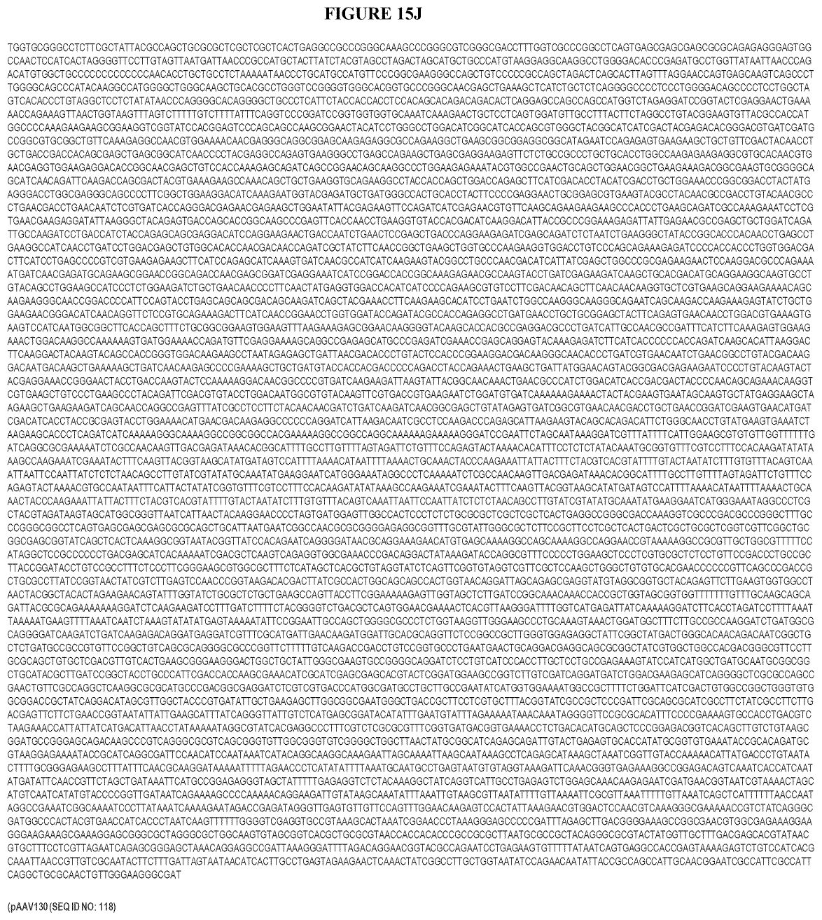

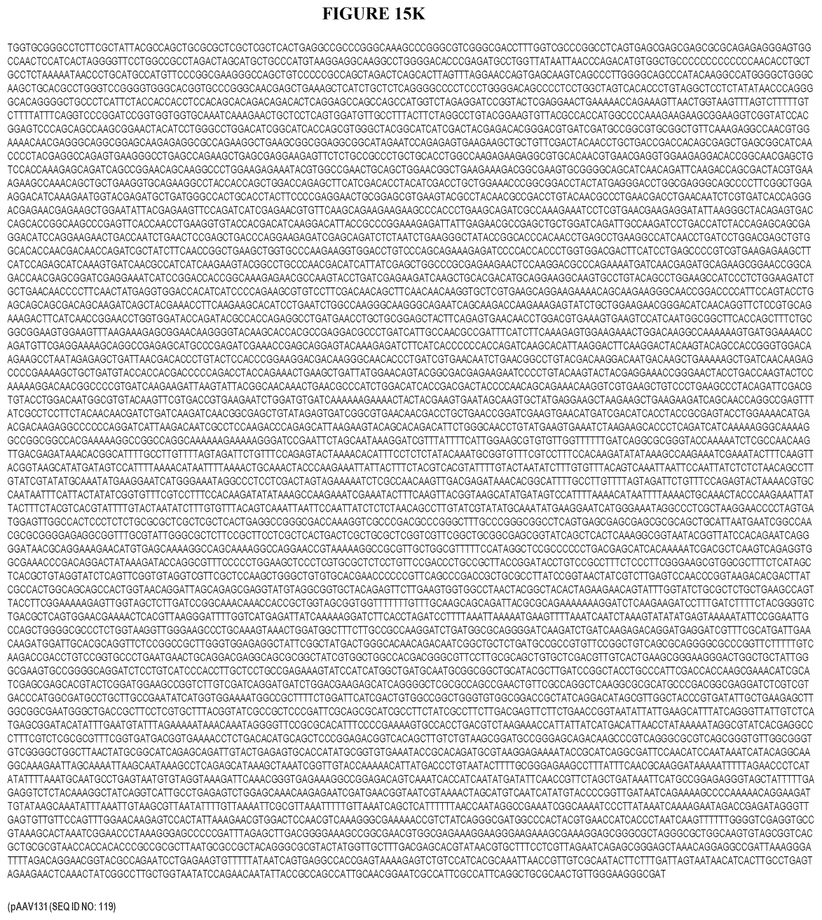

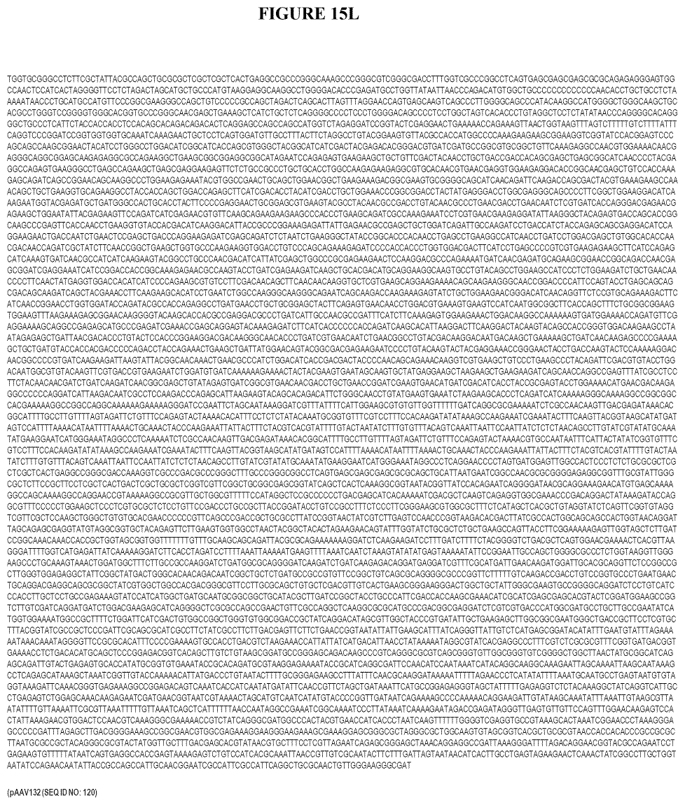

While Construct 30 exhibited only 11% of full MyoAAV-4E capsids, as determined by SEC-MALS, both Construct 138 and Construct 140 exhibited several fold more full capsids at 26% and 29%, respectively (FIG. 13B). Both smaller constructs also exhibited approximately 2-fold greater gene editing efficiency than Construct 30 (FIG. 13C). Additionally, this increased gene editing efficiency over Construct 30 was observed over a range of tested MOIs (FIG. 14).

Collectively, these experiments demonstrated that the smaller genetic constructs exhibited a greater proportion of full capsids in two different AAV vectors. This disparity occurred despite all constructs being produced at similar titers, suggesting that the size-reduced constructs were more efficiently packaged into AAV vectors. Furthermore, the smaller constructs were also capable of editing the mutational hotspot within the dystrophin gene at a higher rate in DMD disease models, suggesting potential synergy between efficient encapsidation and gene expression.

### i. Example 9: Treatment of DMD In Vivo (Mouse Model)

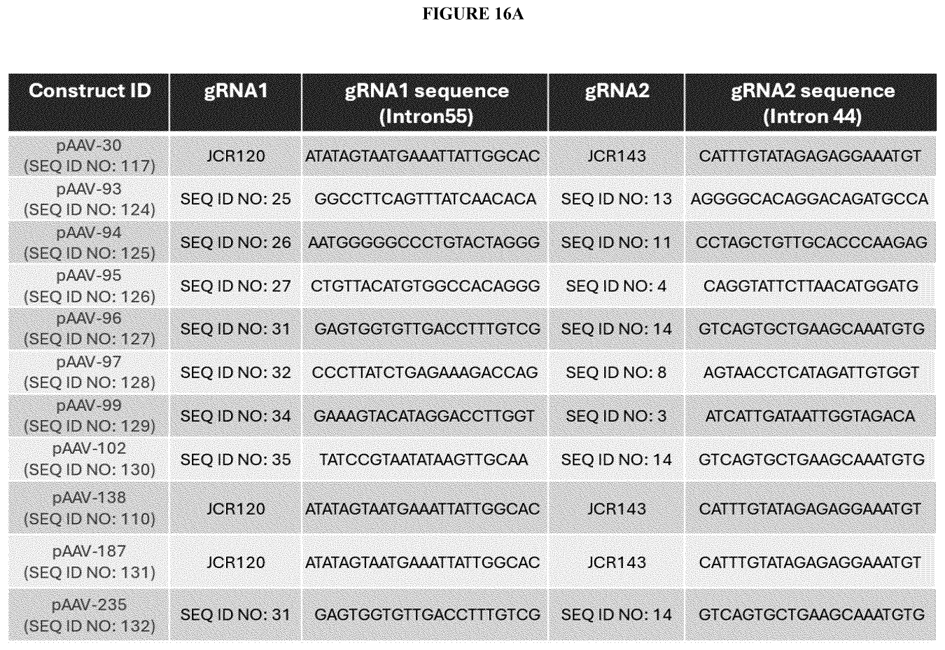

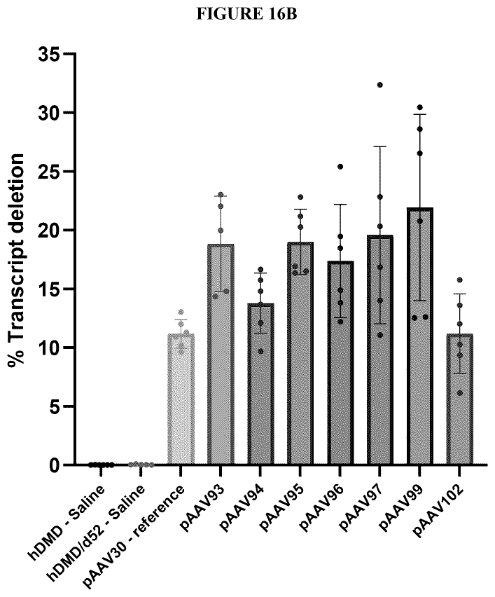

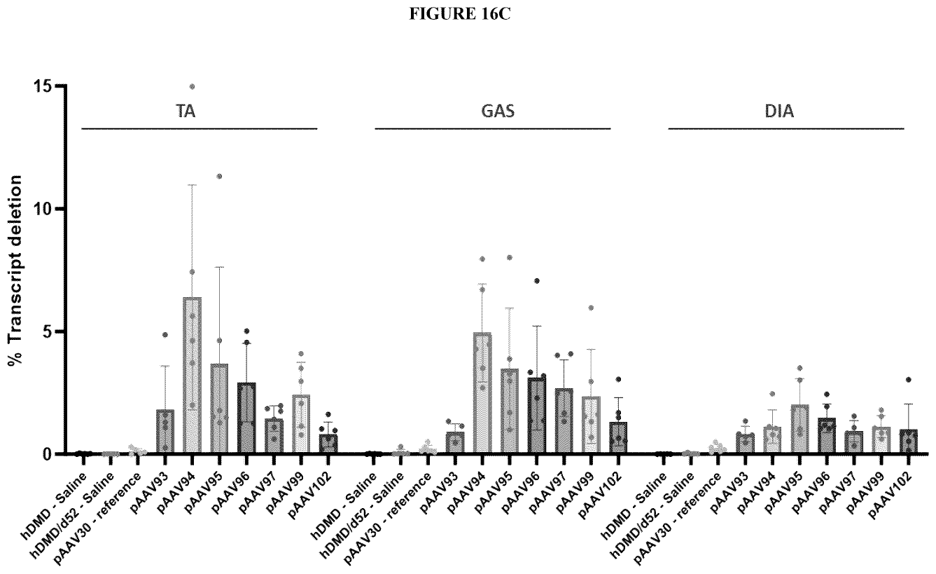

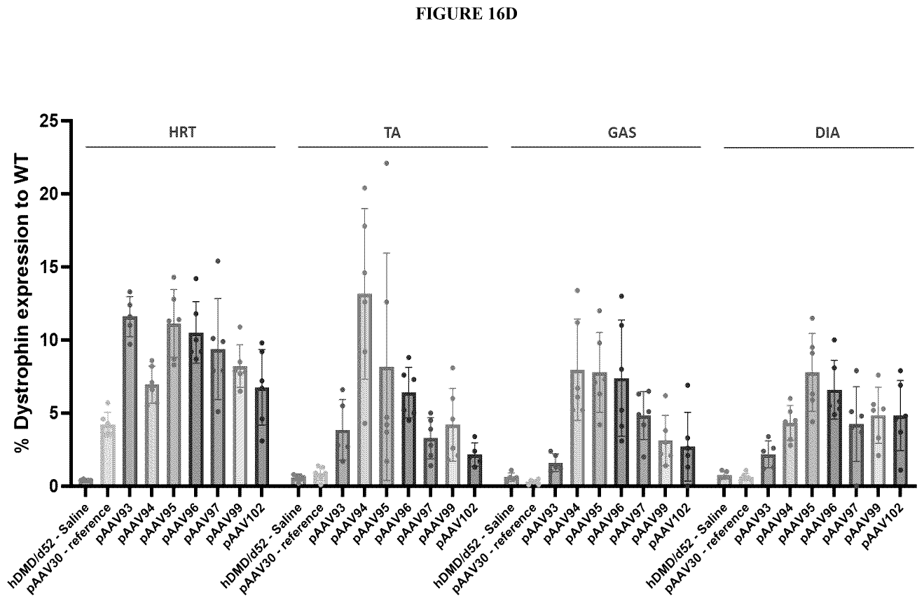

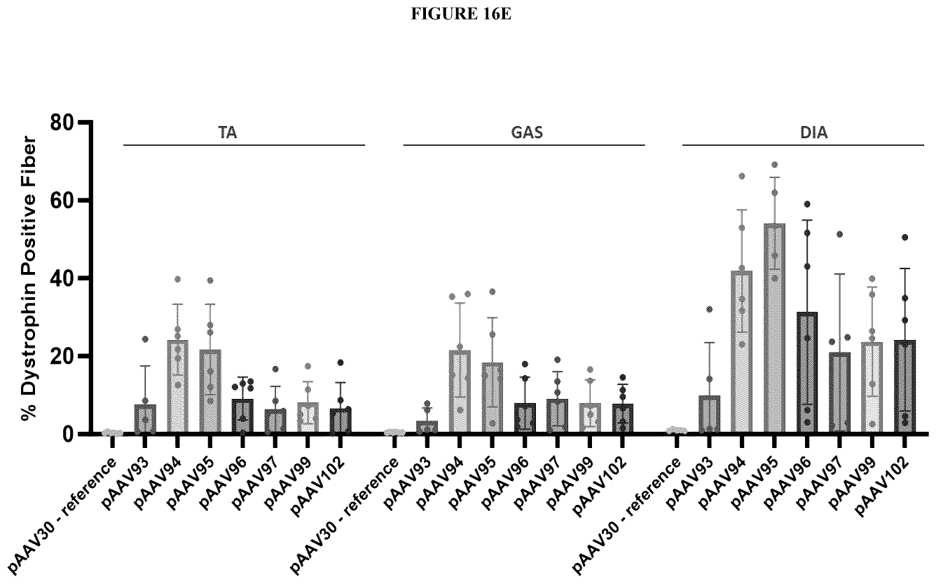

Having observed 1) the high efficiency of CRISPR-mediated deletion of the genomic region of interest in a cell-based system with the presently disclosed gRNAs as compared to benchmark controls and 2) the enhancement of gene editing with benchmark control gRNAs within optimized genetic constructs, the next step was to combine these, such that the presently disclosed gRNAs were encoded within the presently disclosed genetic constructs. Constructs tested are described in FIG. 16A, and the experimental protocol is outlined in Table 7. Briefly, mice were intravenously injected with the indicated test article at 2.5×10¹⁴ viral genomes per kilogram before being sacrificed at 12 weeks post-injection for various assays.

Edited dystrophin transcripts were detected by RT-ddPCR. RNA was isolated from tissue samples using the NucleoMag RNA Kit on the KingFisher Apex instrument. cDNA was generated from 70-100 ng of RNA with a 2-hour 42° C. incubation step. Transcript editing was analyzed by ddPCR using the 2×ddPCR Supermix for probes (no dUTP) and custom PrimeTime assays to quantify edited and unedited cDNA transcripts in the sample. For A45-55 transcript specific edits, the custom PrimeTime Assay KBSRPT_Assay_01 was designed with a HEX labeled probe against the 445-55 edited cDNA sequence (Forward primer: CTGAGAATTGGGAACATGC (SEQ ID NO: 141); reverse primer: CATCGGAACCTTCCAGGG (SEQ ID NO: 142); probe: ACAAATGGTATCTTAAGGACCTCCAAGGTG (SEQ ID NO: 143)). The final primer concentration was 0.25 μM and the final probe concentration was 0.9 μM per reaction. The reference assay used for the 445-55 edit specific master mix was the TaqMan Assay Hs02562862_s1 targeting exon 55 of the DMD transcript and labeled with a VIC-MGB probe. The Hs02562862_s1 assay was diluted to a 1× concentration per reaction. 21 μL of ddPCR master mix was plated into a 96-well PCR plate and 3 μL of cDNA (that was pre-diluted 1:5 in molecular grade H₂O) or water (negative control) was added to bring the final volume up to 24 μL for each respective well. All samples and controls were run in duplicate. 20 μL of the prepared sample plus master mix was then transferred onto a QXOne ddPCR compatible plate (GCR96). The GCR96 plate was then run on a QXOne analyzer following the PCR. Automated positive thresholds were generated for all samples for analysis as possible. Manual thresholds were drawn as needed. Dystrophin protein restoration was quantified by extracting protein from tissue lysate, which was quantified via Nanodrop A280 readings. Samples were normalized to 0.112 ug/μL and loaded according to ProteinSimple's standard protocol. Protein was heated to 95° C. for 5 minutes and then loaded into a 66-440 kDa Fluorescent Separation Module (SM-W008) with the Replex Module (RP-001). Dystrophin was detected with a protein-specific antibody (Abcam ab 154168, 1:600) and an anti-rabbit secondary antibody (DM-001). Alpha-actinin was detected with a protein-specific antibody (Abcam ab254074, 1:50) and an anti-mouse NIR secondary (DM-009). Dystrophin signal was reported as the dystrophin area, with α-actinin serving as a qualitative loading control. All samples without detection of α-actinin were omitted from reporting. A standard curve was included using huDMD/mdx protein lysate. The dystrophin signal area was used to calculate percent expression compared to wildtype. Immunofluorescence was used to quantify dystrophin-positive muscle fibers by sectioning frozen tissue blocks according to standard operating procedures in a cryostat before being affixed to glass microscope slides. Slides were stored in a freezer set to maintain −40° C.±5° C. until staining was performed. Slides were stained according to standard operating procedures for immunofluorescence staining. Antibodies targeting Dystrophin/Laminin and SaCas9/Laminin were applied according to Table 8, with DAPI as a counterstain. Whole slides were scanned by a Leica Aperio VERSA 200 Imaging system according to standard operating procedures. Brightfield calibration of the Aperio Versa 200 system was performed and the “All Routine Scanning Template” was used for image capture. Images underwent a preanalytical quality check. Image analysis was performed using HALO IA algorithms for morphometric assessments.

These assays found that all tested gRNA pairs were able to edit dystrophin, such that varying levels of protein expression were restored. Particularly, administration of all tested gRNA pairs resulted in at least 10% dystrophin transcript editing in the heart muscle, with several outperforming the reference control gRNA pair (FIG. 16B). Additionally, markedly higher levels of editing, compared to the control, were also observed in various muscle tissues, including the tibialis anterior (TA), gastrocnemius (GAS) and diaphragm (DIA) (FIG. 16C). The observed transcript editing also coincided with elevated dystrophin protein expression (FIG. 16D) and more dystrophin-positive muscle fibers (FIG. 16E) in the same tissues. In summary, these tests demonstrated that all seven gRNA pairs were capable of in vivo transcript editing at levels superior to the control gRNAs.

### j. Example 10: Testing Optimized Genetic Constructs with Myospreader Cas9

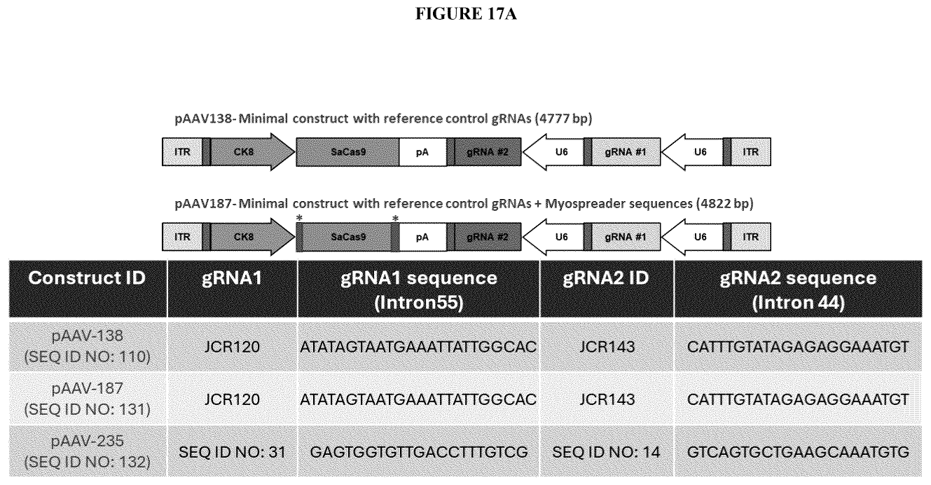

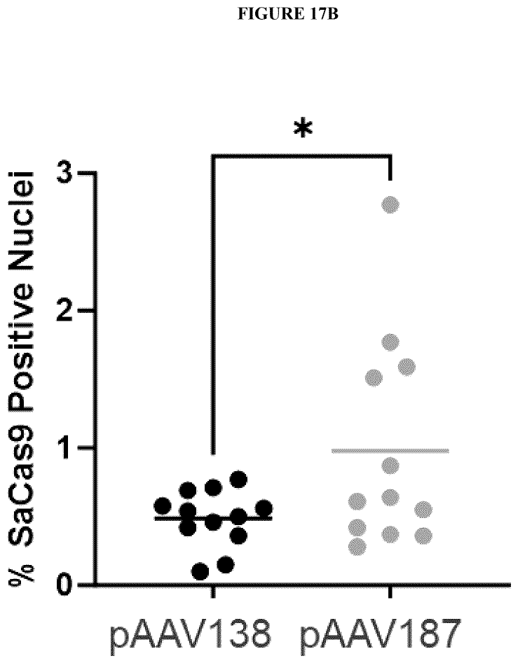

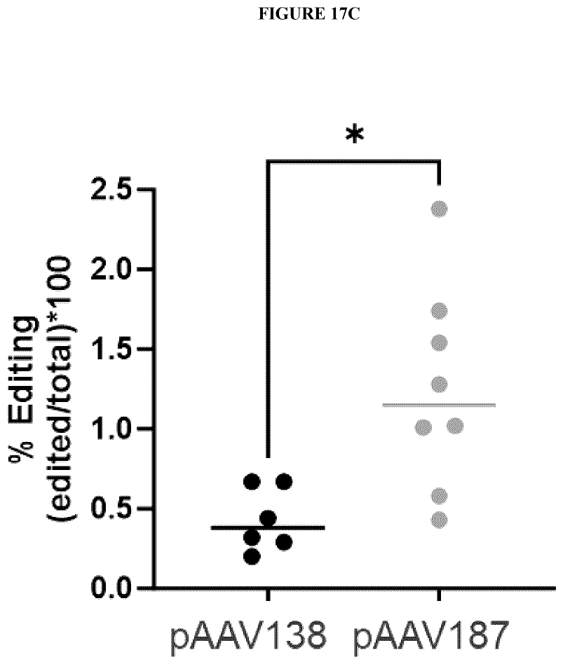

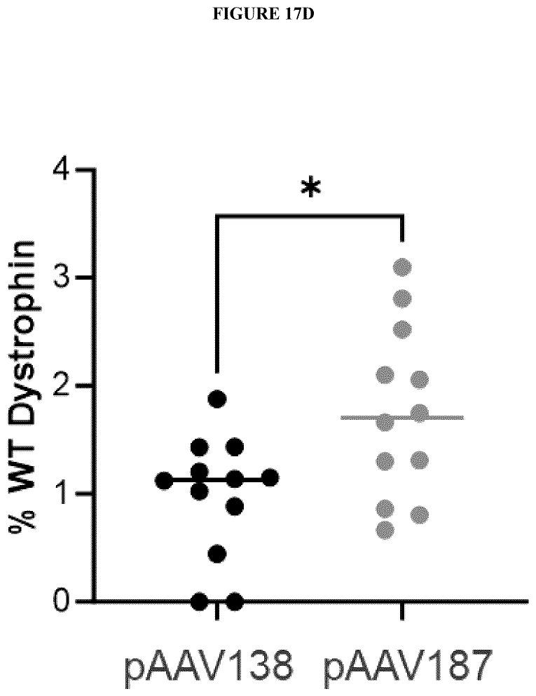

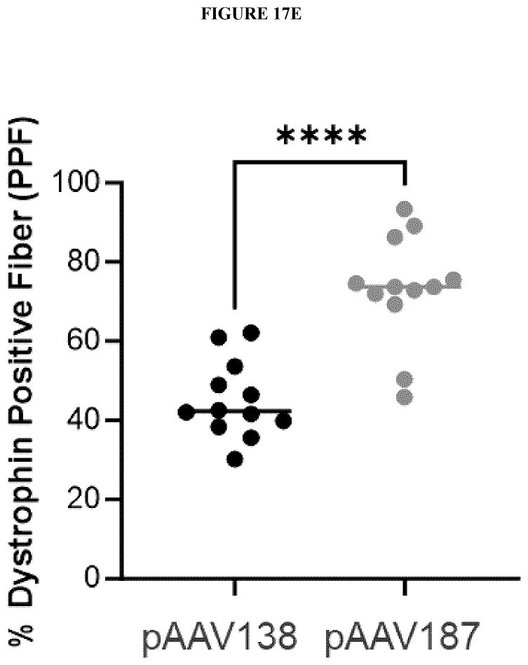

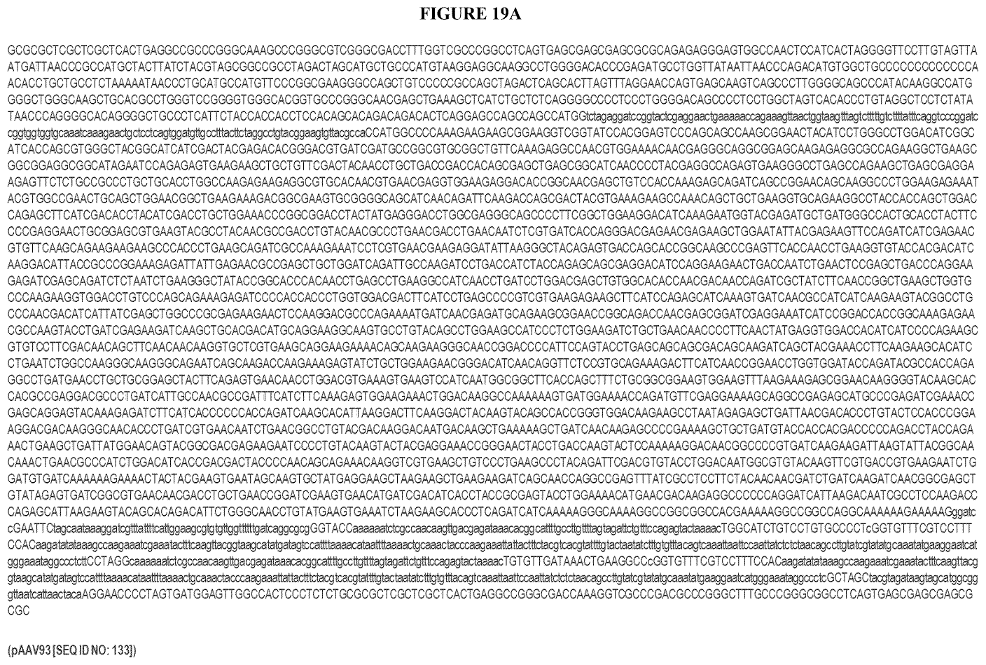

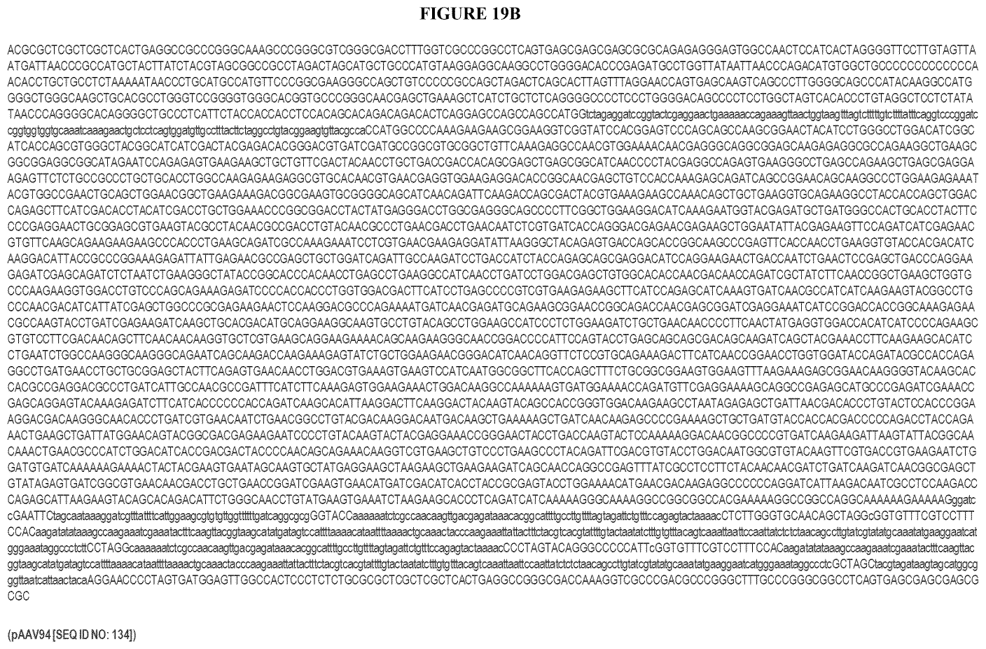

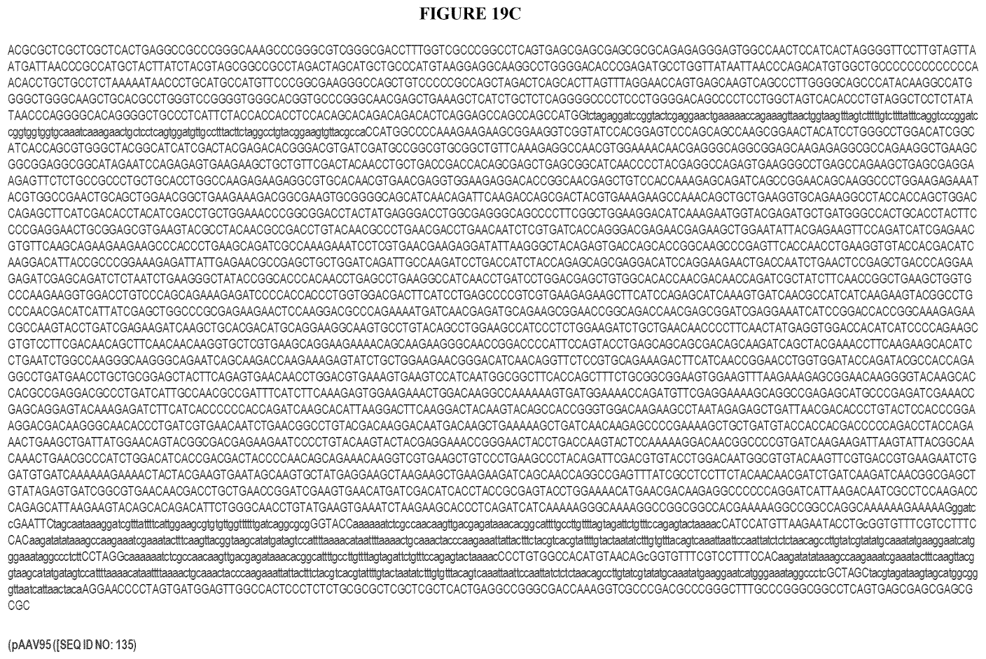

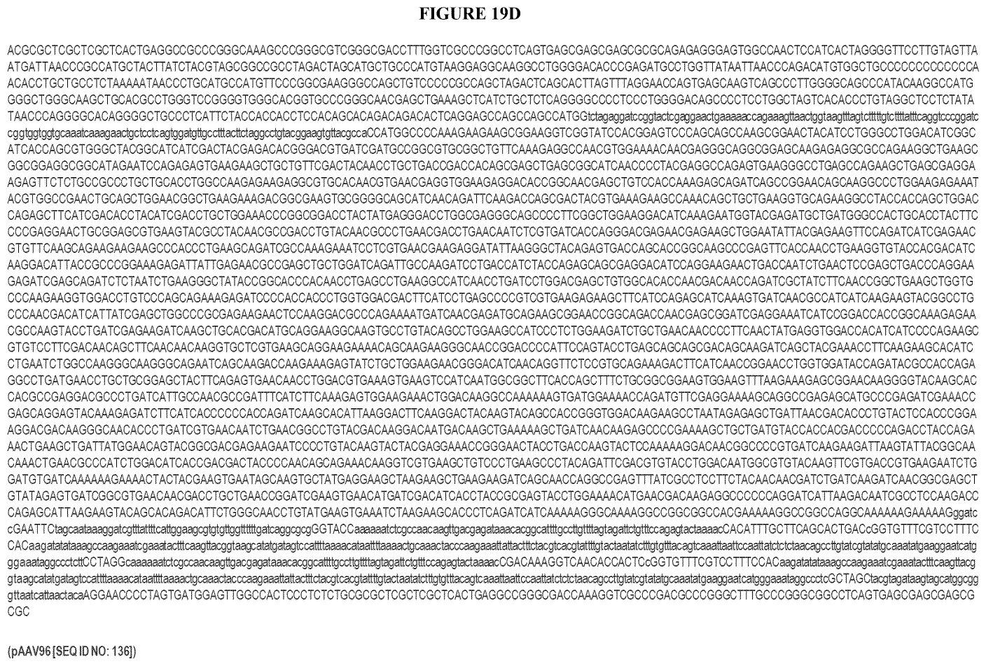

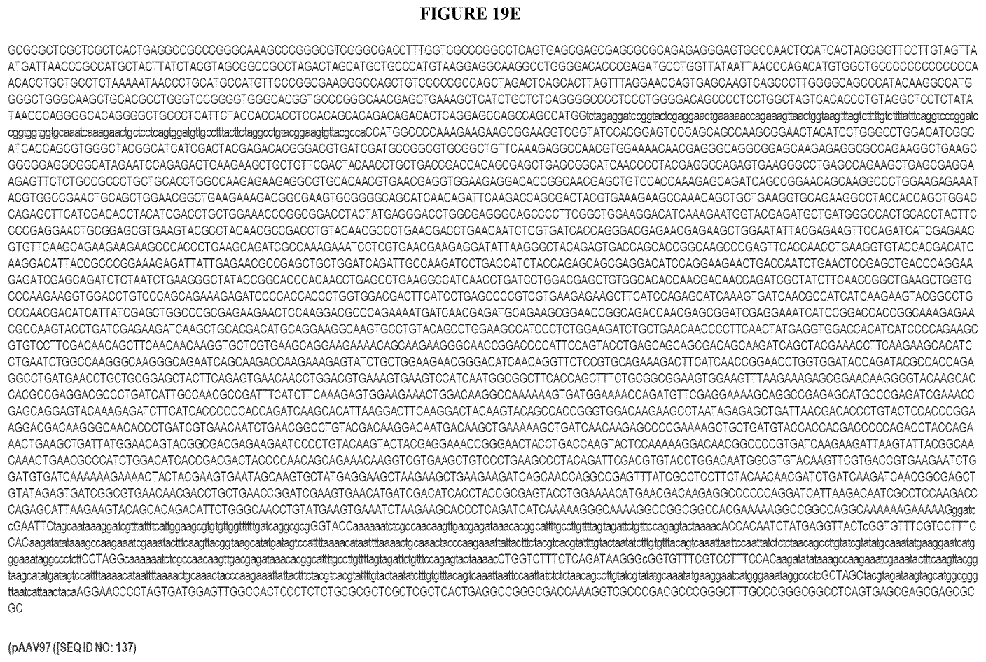

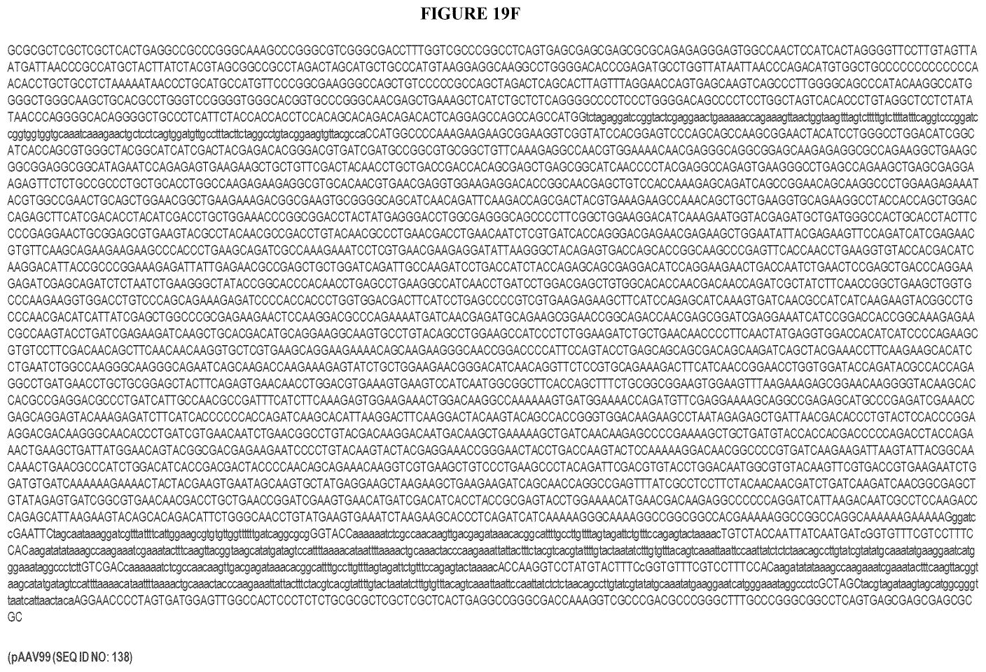

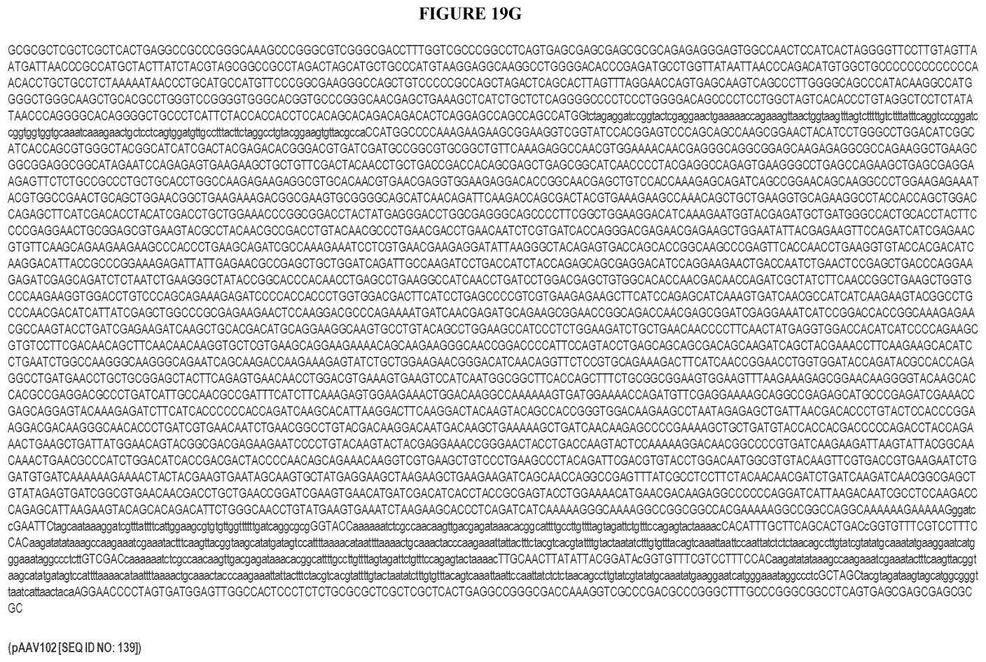

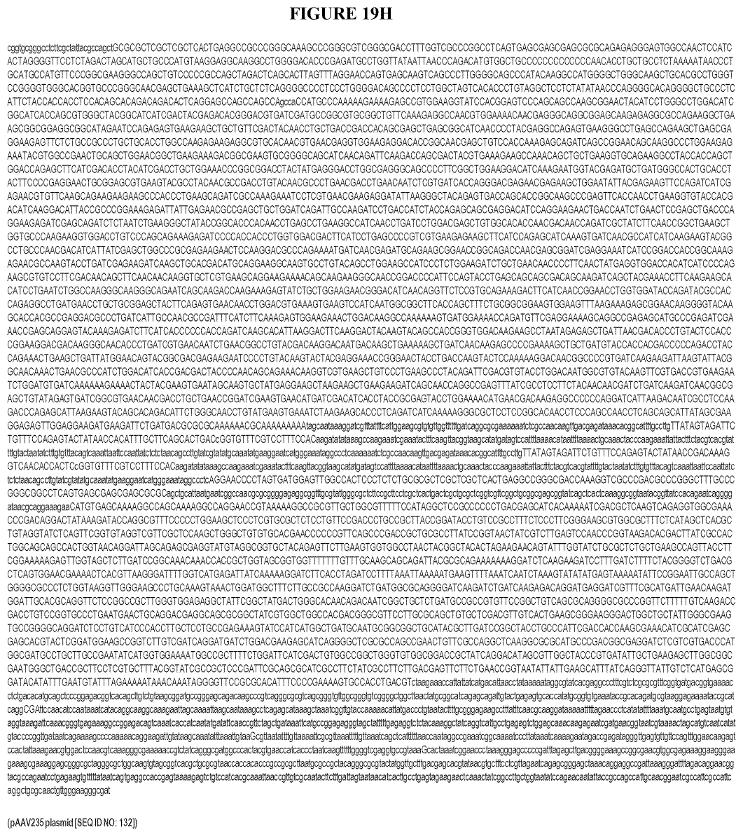

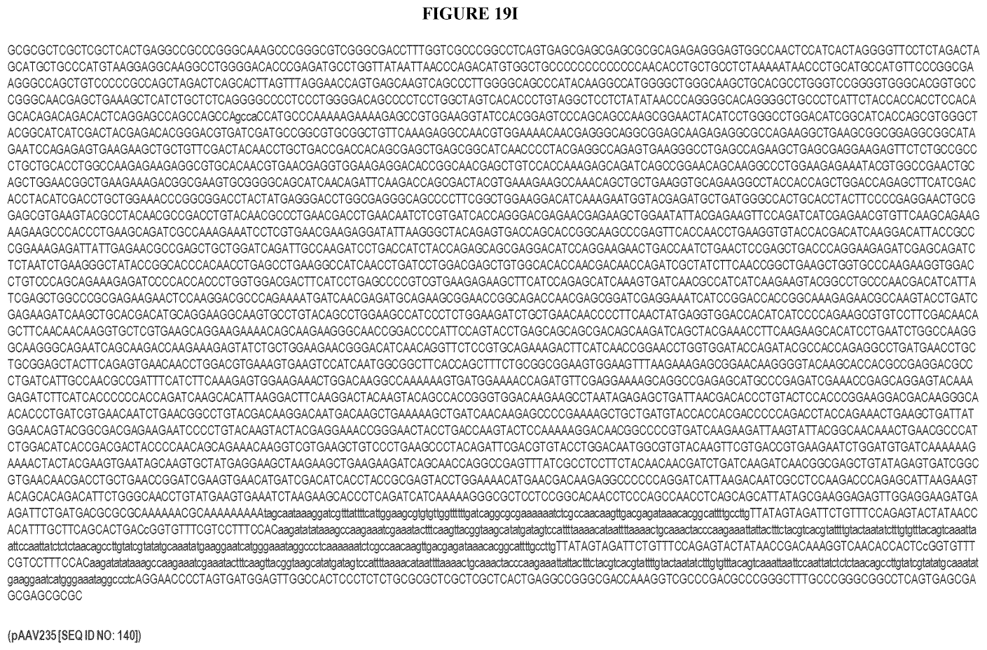

Having observed the impact of the presently disclosed genetic constructs and gRNA pairs on dystrophin gene editing, a next step was to test the impact of incorporating a myospreader Cas9 (see, e.g., Poukalov, Kiril K., et al. “Myospreader improves gene editing in skeletal muscle by myonuclear propagation.” Proceedings of the National Academy of Sciences 121.19 (2024): e2321438121.); exemplary constructs are described in FIGS. 17A and 19I. Briefly, immunofluorescence, RT-ddPCR and protein quantification assays were performed as described in Example 9 above following administration of AAVrh74 vectors containing the reference control gRNAs with SaCas9 (pAAV138) or myospreader SaCas9 (pAAV187) at a dose of 6.3×10¹² viral genomes injected intramuscularly via the GAS muscle.

The assays demonstrated that incorporation of a myospreader Cas9 into the previously described genetic constructs expressing the reference control gRNA pair resulted in significantly greater amounts of SaCas9-positive nuclei (FIG. 17B), significantly elevated levels of dystrophin transcript editing (FIG. 17C), and significantly more dystrophin protein expression (FIG. 17D) and dystrophin-positive muscle fibers (FIG. 17E).

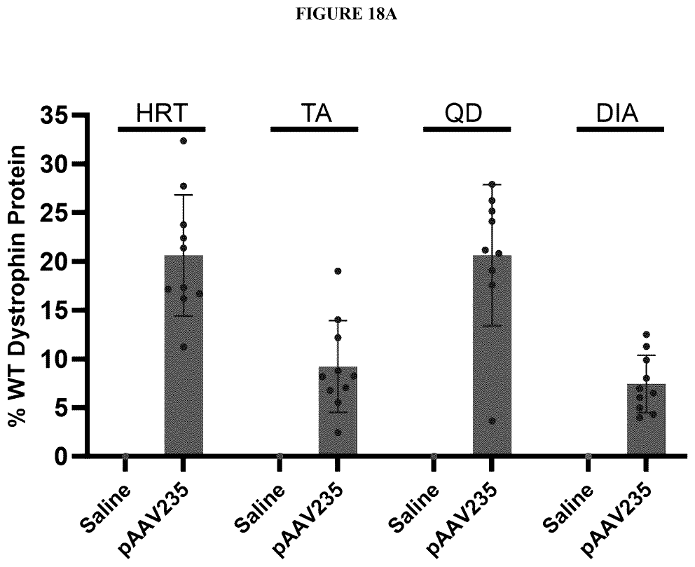

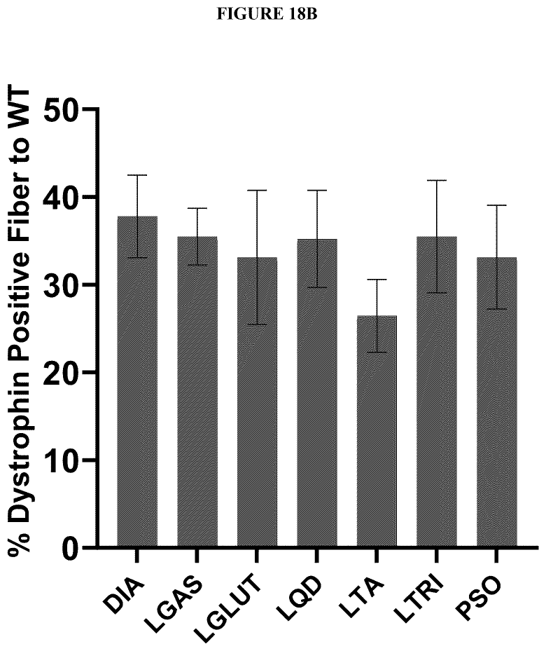

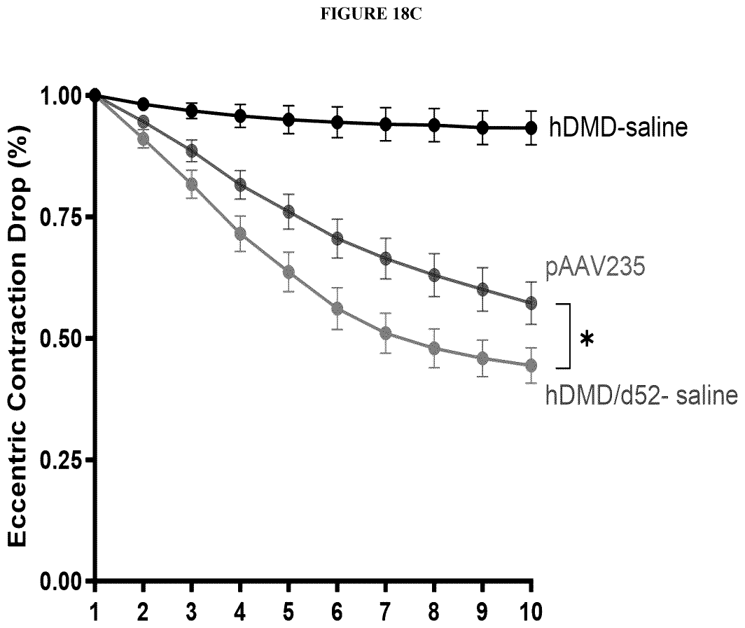

Given the strong results with the reference control gRNA pair, myospreader SaCas9 was also incorporated into a genetic construct expressing a gRNA pair of the present disclosure (pAAV235 [FIGS. 17A, 19I]), which was packaged into an AAVrh74Myo vector and administered intravenously vivo as previously described at 9×10¹³ viral genomes. Treatment with this article resulted in strong dystrophin protein expression (FIG. 18A) and approximately 30-40% of muscle fibers being dystrophin-positive across various tissues (FIG. 18B).

Notably, muscle function was also aided by administration with this article. Briefly, in situ TA physiology was performed as a terminal procedure, at minimum 3 days after the completion of all other outcomes. Mice were anesthetized with an intraperitoneal dose of dilute ketamine and xylazine following standard operating procedures. Once an anesthesia plane was observed, the TA tendon was exposed. A loop was tied to the tendon with a second securing know to prevent slipping. The mouse's knee and foot were secured on the platform under a heat lamp. Electrodes were inserted into the sciatic nerve and instant stimulation was performed to confirm proper placement. The bi-phase stimulator was set to 10 mN. After warm-ups and determination of optimal length, the muscle length was recorded. Force frequencies and eccentric contraction (ECC) were then performed. Data was collected from both TA muscles. Following the in-situ procedure, the animal was given a lethal intraperitoneal dose of ketamine/xylazine cocktail following standard operating procedures. The TA muscles were then extracted and the muscle weight recorded for force normalization.

While no drop in eccentric contraction was observed for the control mouse strain, a greater than 50% drop was observed in the diseased animal; however, treatment with pAAV235 significantly reduced this drop (FIG. 18C). In summary, administration of the gRNA pair and SaCas9 expressed in this genetic construct was able to significantly improve muscle function while also markedly restoring dystrophin protein.

### k. Example 11: Exemplary Methods for AAV Vector Production and Purification

For both dual-vector and single-polynucleotide approaches, AAV vectors (e.g., rh74 or MyoAAV-4E vectors) used in these studies were produced by methods and processes generally known to those of ordinary skill in the art (see, e.g., Rabinowitz, J. E., et al. (2002). Journal of Virology, 76 (2), 791-801.). Briefly, three plasmids (the Cas9- and/or gRNA-containing expression cassette(s) and their respective promoters, AAV capsid genes, and helper adenovirus genes) were co-transfected into HEK293 cells. The three plasmids plus the adenoviral E1 genes in HEK293 cells provided necessary and sufficient elements to produce a large quantity of the viral vectors. The viruses were then either assessed for packaging efficiency by SEC-MALS and/or ultracentrifugation as part of a purification and formulation process prior to use in either in vitro or in vivo studies.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

TABLE 1
Listing of gRNA targeting sequences
in the DMD gene

| SEQ ID | |
|---|---|
| NO: | Protospacer Sequence |
| 1 | CAAAAGGAGCCAGGCUAGAAG |
| 2 | GCAUUCUCACACUAUAAAUGA |
| 3 | AUCAUUGAUAAUUGGUAGACA |
| 4 | CAGGUAUUCUUAACAUGGAUG |
| 5 | GUUUACUCAGUACCCACCUGA |
| 6 | AUGAUCAUUGAGACUACCCAC |
| 7 | GGUAUCCUUGAGUCUUACUGU |
| 8 | AGUAACCUCAUAGAUUGUGGU |
| 9 | CUUGUACAAAAAUGAUGAGUG |
| 10 | GGAUCUGGCCUAGGACAAAUG |
| 11 | CCUAGCUGUUGCACCCAAGAG |
| 12 | AGCCCUUUGAAGAUCAUAGCC |
| 13 | AGGGGCACAGGACAGAUGCCA |
| 14 | GUCAGUGCUGAAGCAAAUGUG |
| 15 | CACAUUUAAAGAGCUAAGCCU |
| 16 | CAGGGAUAAUAAAAGAAGGGA |
| 17 | ACUGUGGGGGCACAAUUAGGA |
| 18 | AAUACAAAGCUAGGUUUAGGG |
| 19 | GCCCAGUUUAGCAAACAGGAU |
| 20 | CAGGUUGUAAGAUAAAUGUCC |
| 21 | AGUUCACCUCAGGGACUGAUG |
| 22 | CAUGAAGGGCCAUGUAAGUAG |
| 23 | UUCAGUAAAGAGGGAUAAGGC |
| 24 | ACAGACAUGGGUUACAUGGGU |
| 25 | GGCCUUCAGUUUAUCAACACA |
| 26 | AAUGGGGGCCCUGUACUAGGG |
| 27 | CUGUUACAUGUGGCCACAGGG |
| 28 | UACACCAACCUAUAUUUGACU |
| 29 | AUGACUUAUCAAAGGAUAUGC |
| 30 | UCCUGAAUUGUAGUAAAAGAG |
| 31 | GAGUGGUGUUGACCUUUGUCG |
| 32 | CCCUUAUCUGAGAAAGACCAG |
| 33 | GAACCUCAAAGAGAGGAGGCC |
| 34 | GAAAGUACAUAGGACCUUGGU |
| 35 | UAUCCGUAAUAUAAGUUGCAA |
| 36 | GUAGCCCCCUGCAGUAUCUAG |
| 37 | AGUGUCAUCAGAAUAUGAUGC |
| 38 | CAUGGACCAAGCCCAAAGUGA |
| 39 | GUAAGUCAUAAUUUCAUAAGC |
| 40 | GCCCUUGCCUAUUCAACAAGC |
| 41 | GCUGCUACCCAAGUUAUCCUG |
| 42 | UUCUACCCCUGCCUUAGAAGG |
| 43 | UGAGUCCAAUCCAUGAUCCAG |
| 44 | UGAUCCAGCAGAGUUUCAGCC |
| 45 | AUUGUAAUAGCUAGUUAGAGG |
| 46 | CAGACCCAAACAGUAAGACUC |
| 47 | AGAUUGCAUGGAUUUAAUGUG |
| 48 | GGUUGUGCAUACUAAUUGCCA |
| 49 | GAAGCCUUAGUAAGAUCAGAG |
| 50 | UGUUUGAAGCCUUAGUAAGAU |
| 51 | CUUCGGGUUCAGUUGAAAGCA |
| 52 | GACAGGGAUUUGACAAAAAUG |
| 53 | AGAGUUAGAACACUAGCAAGG |
| 54 | GAUAAGCAGUGGUGCCAAGGG |
| 55 | AGGAGAUAAGCAGUGGUGCCA |
| 56 | GGGCUGAAACUCUGCUGGAUC |
| 57 | AAAUCCUGGGCUGAAACUCUG |
| 58 | AAGGCAGCAGAAUUUAGCCAC |
| 59 | CACAUAUAUGGCAUAAAAGCC |
| 60 | AGUUGUUAUUAGAGAUGAUGU |
| 61 | CCUGCAUAAACUUUGAGAGGG |
| 62 | UGUCUACCAAUUAUCAAUGAU |
| 63 | AGCAUGGUGUCAGAAGGAACU |
| 64 | CUCUCGAUAGAAGCUAAAGUG |
| 65 | GAUAAUGAAAAAUUGGGACCU |
| 66 | UAGUGUUAAGUUGAACGAUUG |
| 67 | GCACUUCUGUAUGAGAGGCUG |
| 68 | CAGGUUGUAAGAUAAAUGUCC |
| 69 | AGUUCACCUCAGGGACUGAUG |
| 70 | GCUUCACAAUGAUCAGGGUGG |
| 71 | GCUUCACAAUGAUCAGGGUGG |
| 72 | AGGGUGGAAUGAAGAGCUCUG |
| 73 | GAUAAGGCUUGAGUGGUCACA |
| 74 | CAGAGUUUUAAAAGAUGUGCU |
| 75 | UGACAAGCAAGUUCAUUUGUU |
| 76 | AAGCCCAAGCCCCGCCAACAG |
| 77 | UAAGAGUAGCAGAGCAUUUAG |
| 78 | GAAGUUAGAUGAAGAAAGACG |
| 79 | CCUAUAUUUGACUCAGGGUUG |
| 80 | AACAAUAUCUUGACACCACCA |
| 81 | AAACGGACAGGUGAAAGCUGA |
| 82 | UGGGUGGGUAACCAUGGAGAA |
| 83 | UUCUGGCUUAGAGAAGAAGAU |
| 84 | UUUGAUCCACCCUUCUAAGGC |
| 85 | ACAGUGUAGCCAAUGAGAGCU |
| 86 | UCCUCAGAGACUAUUAUCCUG |
| 87 | UUUGGUUUAUGAGCAGGGACA |
| 88 | UGUUACCUUGGAUUUUCAAAG |
| 89 | GUUGGUUUUAUUAUGAGGUGG |
| 90 | AUUGAAGCCUUGACAUAACUU |
| 91 | AUAGGUAUCACCUAUGGAUGC |
| 92 | CAGAGUUAAAAUACUGACUUG |
| 93 | UUCUCAUGUAAACAGUCUAAG |
| 94 | AAGGUACUUCUGACUUUCCAA |
| 95 | AAAGGGCCAUAGUAGCAGAAG |
| 96 | GCUUUUCUACCCCUGCCUUAG |
| 97 | CCUUUACACAAGAAGCUACAG |
| 98 | GUACUUCACAGAGCCAGGGGG |

TABLE 2
Characteristics associated with disclosed
gRNA pairs targeting the DMD gene

| Intron 44 | Intron 55 | | |
|---|---|---|---|
| gRNA (SEQ | gRNA (SEQ | Genomic deletion | Resulting synthetic |
| ID NO:) | ID NO:) | size (bases) | intron size (bases) |
| 4 | 20 | 626509 | 82956 |
| 8 | 20 | 580377 | 129088 |
| 4 | 21 | 630840 | 78625 |
| 13 | 21 | 640444 | 69021 |
| 1 | 22 | 653297 | 56168 |
| 8 | 22 | 590882 | 118583 |
| 3 | 23 | 638824 | 70641 |
| 17 | 24 | 606326 | 103139 |
| 13 | 25 | 651307 | 58158 |
| 3 | 26 | 643577 | 65888 |
| 11 | 26 | 656767 | 52698 |
| 13 | 26 | 651416 | 58049 |
| 17 | 27 | 616100 | 93365 |
| 4 | 27 | 649156 | 60309 |
| 7 | 27 | 604833 | 104632 |
| 9 | 28 | 670139 | 39326 |
| 4 | 29 | 651654 | 57811 |
| 1 | 30 | 669479 | 39986 |
| 2 | 31 | 676323 | 33142 |
| 4 | 31 | 669319 | 40146 |
| 8 | 31 | 623187 | 86278 |
| 13 | 31 | 678923 | 30542 |
| 14 | 31 | 676514 | 32951 |
| 15 | 31 | 654641 | 54824 |
| 16 | 31 | 646109 | 63356 |
| 18 | 32 | 628597 | 80868 |
| 1 | 32 | 689816 | 19649 |
| 8 | 32 | 627401 | 82064 |
| 1 | 33 | 699954 | 9511 |
| 3 | 34 | 685782 | 23683 |
| 4 | 34 | 684017 | 25448 |
| 7 | 34 | 639694 | 69771 |
| 11 | 35 | 646556 | 62909 |
| 14 | 35 | 638796 | 70669 |
| 19 | 36 | 582210 | 127255 |
| 6 | 36 | 610509 | 98956 |
| 18 | 37 | 590553 | 118912 |
| 10 | 38 | 657226 | 52239 |
| 5 | 39 | 633538 | 75927 |
| 4 | 40 | 647183 | 62282 |
| 3 | 41 | 667888 | 41577 |
| 1 | 41 | 682406 | 27059 |
| 8 | 41 | 619991 | 89474 |
| 10 | 41 | 682587 | 26878 |
| 3 | 42 | 672239 | 37226 |
| 12 | 42 | 681917 | 27548 |

TABLE 3
Exemplary CRISPR nucleases and minimum requirements for gene editing function

| Nuclease (bacterial | Size | PAM | Nucleic acid target | Protospacer |
|---|---|---|---|---|
| species of origin) | (aa) | (5′→3′) | (end type generated) | length (nt) |
| Cas9 (S. pyogenes) | 1368 | NGG | dsDNA (blunt) | 20 |
| Cas9 (S. aureus) | 1053 | NNGRRT | dsDNA (blunt) | 20 |
| Cas9 (S. thermophilus) | 1122 | NNAGAAW | dsDNA (blunt) | 20 |
| Cas9 (N. meningitidis) | 1083 | NNNNGATT | dsDNA (blunt) | 24 |
| Cpf1 (F. novicida) | 1300 | (T)TTV | SSDNA/dsDNA | 23-25 |
| | | | (staggered) | |
| Cpf1 (Lachnospiraceae | 1228 | TTTV | SSDNA/dsDNA | 23-25 |
| sp.) | | | (staggered) | |
| Cpf1 (Acidaminococcus | 1307 | TTTN | SSDNA/dsDNA | 24 |
| sp.) | | | (staggered) | |
| Cas12f (A. sulfuroxidans) | 422 | NTTR | dsDNA (staggered) | variable |

See generally, Wang, J., et al. (2020). Journal of Cellular and Molecular Medicine, 24(6), 3256-3270; Liu, G., et al. (2022). Molecular Cell, where N=any nucleotide, R = any purine, Y = any pyrimidine, W = A or T, and V = A, C or G.

TABLE 4
Quantification of results from arrayed
screen of filtered gRNA pairs

| gRNA pair | | Average | | |
|---|---|---|---|---|
| Intron 44 | Intron 55 | deletion | | |
| gRNA (SEQ | gRNA (SEQ | efficiency | | Array |
| ID NO:) | ID NO:) | (%) | SEM | Number |
| No treatment | | 0.022984884 | 0.002264 | 1 |
| 4 | 20 | 31.94936016 | 1.394161 | 1 |
| 8 | 20 | 34.2276659 | 1.940755 | 1 |
| 4 | 21 | 15.92867703 | 3.825143 | 1 |
| 1 | 21 | 28.7217873 | 1.894241 | 1 |
| 57 | 21 | 1.993550026 | 0.674547 | 1 |
| 57 | 70 | 0.850033292 | 0.125756 | 1 |
| 64 | 72 | 27.62356613 | 1.225894 | 1 |
| 54 | 22 | 18.77313161 | 2.477396 | 1 |
| 1 | 22 | 35.57275624 | 2.017412 | 1 |
| 56 | 22 | 3.457944718 | 0.667083 | 1 |
| 59 | 22 | 21.54524264 | 1.572389 | 1 |
| 8 | 22 | 30.48840565 | 3.137014 | 1 |
| 9 | 22 | 22.62504754 | 1.240499 | 1 |
| 1 | 73 | 29.66381476 | 3.2934 | 1 |
| 3 | 23 | 32.90095081 | 2.061653 | 1 |
| 17 | 24 | 31.72213216 | 1.505612 | 1 |
| 62 | 24 | 8.569498984 | 1.565642 | 1 |
| 13 | 25 | 29.11531535 | 4.357413 | 1 |
| 10 | 75 | 0.03695871 | 0.008793 | 1 |
| 2 | 26 | 23.8742339 | 4.903004 | 1 |
| 46 | 26 | 19.32728857 | 3.231732 | 1 |
| 3 | 26 | 37.79894563 | 1.732668 | 1 |
| 8 | 26 | 30.26977387 | 1.729733 | 1 |
| 11 | 26 | 38.51238573 | 2.604274 | 1 |
| 13 | 26 | 36.70075318 | 2.906013 | 1 |
| 3 | 76 | 18.78806658 | 1.555921 | 1 |
| 57 | 76 | 0.53458186 | 0.120266 | 1 |
| 17 | 27 | 39.64264539 | 2.256047 | 1 |
| 4 | 27 | 35.11144229 | 1.6516 | 1 |
| 54 | 27 | 21.80031993 | 1.136003 | 1 |
| 4 | 35 | 30.65993372 | 1.389976 | 1 |
| 54 | 78 | 19.24585904 | 1.224831 | 1 |
| 1 | 78 | 30.11242993 | 1.233704 | 1 |
| 7 | 78 | 24.22440449 | 1.743854 | 1 |
| 9 | 28 | 32.7818359 | 2.418984 | 1 |
| 5 | 35 | 23.0659944 | 1.657563 | 1 |
| 8 | 35 | 29.26659555 | 0.866445 | 1 |
| No Treatment | | 0.005206877 | 0.003325 | 2 |
| 1 | 30 | 33.52720994 | 0.924227 | 2 |
| 13 | 30 | 26.05287502 | 0.945528 | 2 |
| 15 | 30 | 28.27194566 | 1.077024 | 2 |
| 4 | 37 | 31.24576058 | 0.924552 | 2 |
| 3 | 81 | 23.46748652 | 0.742635 | 2 |
| 11 | 82 | 16.07496497 | 1.203352 | 2 |
| 2 | 31 | 33.46020497 | 1.342949 | 2 |
| 48 | 31 | 24.89914035 | 0.491157 | 2 |
| 4 | 31 | 37.97701894 | 0.87934 | 2 |
| 54 | 31 | 25.96085419 | 1.527397 | 2 |
| 57 | 31 | 0.850981965 | 0.076391 | 2 |
| 59 | 31 | 19.65121669 | 1.663653 | 2 |
| 8 | 31 | 32.7951619 | 1.11594 | 2 |
| 11 | 40 | 23.84426168 | 1.537698 | 2 |
| 14 | 31 | 42.88284495 | 0.783263 | 2 |
| 15 | 31 | 36.48305463 | 0.617024 | 2 |
| 18 | 32 | 34.5012741 | 0.888365 | 2 |
| 54 | 32 | 25.16637976 | 1.193705 | 2 |
| 1 | 32 | 34.72088139 | 1.125646 | 2 |
| 57 | 32 | 0.481723401 | 0.098801 | 2 |
| 8 | 32 | 38.13356162 | 0.782732 | 2 |
| 13 | 32 | 30.41062996 | 2.367777 | 2 |
| 54 | 87 | 0.107557166 | 0.031301 | 2 |
| 7 | 87 | 0.096820077 | 0.04511 | 2 |
| 11 | 87 | 0.26079625 | 0.067264 | 2 |
| 49 | 33 | 24.50389495 | 1.018178 | 2 |
| 1 | 33 | 45.28142164 | 0.993851 | 2 |
| 66 | 33 | 24.52415834 | 0.587699 | 2 |
| 3 | 34 | 42.5941989 | 1.177744 | 2 |
| 4 | 34 | 36.384898 | 1.685609 | 2 |
| 54 | 34 | 30.10586519 | 1.233055 | 2 |
| 56 | 34 | 2.998996454 | 0.505038 | 2 |
| 7 | 34 | 36.36687334 | 1.776805 | 2 |
| 66 | 34 | 30.42270334 | 1.477988 | 2 |
| 17 | 88 | 24.58898995 | 1.404101 | 2 |
| 63 | 35 | 0.031193109 | 0.008518 | 2 |
| 56 | 35 | 2.029831132 | 0.66116 | 2 |
| 59 | 35 | 21.18989885 | 1.053312 | 2 |
| 7 | 78 | 28.47344809 | 0.926301 | 2 |
| 5 | 35 | 24.52016334 | 1.495777 | 2 |
| 8 | 35 | 31.32536866 | 0.999586 | 2 |
| No treatment | | 0.116648 | 0.103019 | 3 |
| Benchmark Controls-1 | | 5.182885 | 0.981114 | 3 |
| Benchmark Controls-2 | | 10.67659 | 0.789068 | 3 |
| 9 | 24 | 25.11728 | 0.825152 | 3 |
| 13 | 78 | 24.38133 | 0.66713 | 3 |
| 6 | 36 | 31.38421 | 0.295532 | 3 |
| 2 | 76 | 7.173265 | 0.32386 | 3 |
| 56 | 38 | 1.69923 | 0.297576 | 3 |
| 5 | 39 | 28.30831 | 0.808113 | 3 |
| 7 | 27 | 31.32399 | 0.401816 | 3 |
| 2 | 93 | 14.14729 | 0.252525 | 3 |
| 63 | 93 | 13.43004 | 0.470322 | 3 |
| 54 | 95 | 15.72576 | 0.430268 | 3 |
| 56 | 95 | 6.853999 | 0.08327 | 3 |
| 10 | 95 | 22.78389 | 0.572529 | 3 |
| 18 | 41 | 26.07492 | 0.690343 | 3 |
| 3 | 41 | 34.77923 | 0.619239 | 3 |
| 1 | 41 | 38.36826 | 0.682426 | 3 |
| 59 | 41 | 25.51123 | 0.627186 | 3 |
| 8 | 41 | 28.12358 | 0.661936 | 3 |
| 10 | 41 | 34.48495 | 0.426519 | 3 |
| 67 | 41 | 26.68389 | 0.459151 | 3 |
| 3 | 42 | 36.57013 | 0.196596 | 3 |
| 64 | 42 | 15.47804 | 0.497441 | 3 |
| 17 | 97 | 27.43386 | 0.306361 | 3 |
| 4 | 98 | 21.19425 | 0.438924 | 3 |
| 10 | 98 | 24.02589 | 1.102132 | 3 |
| 60 | 89 | 12.52209 | 0.498938 | 3 |
| 48 | 34 | 26.02091 | 0.379976 | 3 |
| 16 | 31 | 35.83992 | 0.430678 | 3 |
| 7 | 78 | 19.85646 | 0.812872 | 3 |
| 5 | 35 | 22.67695 | 1.043926 | 3 |
| 8 | 35 | 23.33969 | 0.696968 | 3 |
| No treatment | | 0.043398 | 0.012741 | 4 |
| 13 | 31 | 36.64683 | 0.823452 | 4 |
| 1 | 80 | 8.134667 | 0.486231 | 4 |
| 4 | 29 | 37.34338 | 0.931536 | 4 |
| 1 | 82 | 12.25659 | 0.578707 | 4 |
| 2 | 33 | 26.70245 | 0.528553 | 4 |
| 17 | 36 | 22.53991 | 0.322759 | 4 |
| 14 | 35 | 40.68894 | 0.731136 | 4 |
| 11 | 35 | 34.75087 | 0.897384 | 4 |
| 50 | 90 | 12.30473 | 0.44616 | 4 |
| 19 | 92 | 29.17042 | 0.724593 | 4 |
| 1 | 37 | 0.066997 | 0.015199 | 4 |
| 57 | 37 | 0.434202 | 0.061 | 4 |
| 7 | 37 | 27.39927 | 0.502138 | 4 |
| 15 | 37 | 25.45729 | 0.296953 | 4 |
| 48 | 38 | 19.5014 | 0.393462 | 4 |
| 10 | 38 | 28.09464 | 0.198155 | 4 |
| 67 | 38 | 23.45767 | 0.128453 | 4 |
| 18 | 92 | 22.25047 | 0.311814 | 4 |
| 47 | 40 | 26.43824 | 0.274049 | 4 |
| 4 | 40 | 28.29743 | 0.744433 | 4 |
| 12 | 42 | 29.46398 | 0.298904 | 4 |
| 13 | 21 | 34.94772 | 0.795319 | 4 |
| 2 | 72 | 20.68475 | 0.30953 | 4 |
| 54 | 38 | 16.54277 | 0.315348 | 4 |
| 14 | 76 | 24.72878 | 0.332575 | 4 |
| 2 | 78 | 25.90967 | 0.439924 | 4 |
| 2 | 95 | 13.56427 | 0.265713 | 4 |
| 18 | 37 | 32.1661 | 0.677387 | 4 |
| 63 | 97 | 21.64344 | 0.276443 | 4 |
| 56 | 27 | 0.991025 | 0.093909 | 4 |
| 15 | 78 | 20.88784 | 0.076034 | 4 |
| 14 | 79 | 26.92517 | 0.502961 | 4 |
| 67 | 35 | 20.14303 | 0.069386 | 4 |
| 5 | 35 | 12.69409 | 0.308237 | 4 |
| 51 | 81 | 22.65182 | 0.6111 | 4 |
| 17 | 34 | 26.22282 | 0.636481 | 4 |

TABLE 5
Quantification of results from gRNA pair screening validation

| gRNA pair | | Average | |
|---|---|---|---|
| Intron 44 gRNA | Intron 55 gRNA | deletion | |
| (SEQ ID NO:) | (SEQ ID NO:) | efficiency (%) | SEM |
| 3 | 20 | 55.38908574 | 2.082471 |
| 8 | 20 | 55.30604887 | 1.903663 |
| 13 | 21 | 58.79639112 | 1.28025 |
| 1 | 22 | 52.08803473 | 1.062486 |
| 8 | 22 | 49.48318527 | 1.317037 |
| 3 | 23 | 54.15662323 | 0.960469 |
| 17 | 24 | 52.64336052 | 1.32292 |
| 13 | 25 | 58.43693714 | 1.35768 |
| 3 | 26 | 54.32306396 | 1.592481 |
| 11 | 26 | 58.24632691 | 0.954353 |
| 13 | 26 | 52.62881847 | 1.791289 |
| 17 | 27 | 48.29086532 | 1.128827 |
| 4 | 27 | 60.99344126 | 1.486342 |
| 9 | 28 | 45.96226718 | 1.226114 |
| 1 | 31 | 48.0154646 | 1.522212 |
| 2 | 31 | 46.97555014 | 1.018768 |
| 4 | 31 | 53.13392521 | 1.440338 |
| 8 | 31 | 50.01786337 | 0.848844 |
| 14 | 31 | 60.29510052 | 0.550511 |
| 15 | 31 | 48.09829485 | 1.741285 |
| 18 | 32 | 51.66800682 | 0.525015 |
| 1 | 32 | 52.74416012 | 0.879081 |
| 8 | 32 | 57.95262333 | 0.478183 |
| 1 | 33 | 63.39307309 | 1.705164 |
| 3 | 34 | 58.27070063 | 1.57703 |
| 4 | 34 | 50.17088173 | 0.916086 |
| 7 | 34 | 44.90209559 | 1.074616 |
| 6 | 36 | 35.83983897 | 1.186278 |
| 5 | 39 | 32.2691919 | 1.579351 |
| 7 | 7 + 27 | 44.79603745 | 1.524776 |
| 3 | 3 + 41 | 49.51894523 | 0.930254 |
| 1 | 1 + 41 | 58.55412429 | 1.0881 |
| 8 | 8 + 41 | 39.77118765 | 1.37414 |
| 10 | 10 + 41 | 43.94613392 | 1.424292 |
| 3 | 42 | 52.75751896 | 2.951848 |
| 16 | 31 | 52.96584535 | 1.500088 |
| 13 | 31 | 57.18195321 | 1.781578 |
| 4 | 29 | 57.59332459 | 1.67269 |
| 14 | 35 | 57.95739153 | 0.995191 |
| 11 | 35 | 53.77495308 | 0.854482 |
| 19 | 36 | 46.12165847 | 1.215851 |
| 10 | 38 | 45.84813758 | 1.894206 |
| 4 | 40 | 52.16690956 | 1.481309 |
| 12 | 42 | 45.62904672 | 2.613586 |
| 13 | 21 | 56.56473462 | 1.208958 |
| 18 | 37 | 49.3088656 | 1.731333 |

| | | |
|---|---|---|
| No treatment | 0.067593249 | 0.034541 |
| Benchmark controls | 19.79411953 | 1.4911 |

TABLE 6
Listing of exemplary vector components and other nucleotide sequences

| Component | Sequence |
|---|---|
| ITR sequence | GCGCGCTCGCTCGCTCACTGAGGCCGCCCGGGCAAAGCCCGGG |
| (SEQ ID NO: 99) | CGTCGGGCGACCTTTGGTCGCCCGGCCTCAGTGAGCGAGCGAG |
| | CGCGCAGAGAGGGAGTGGCCAACTCCATCACTAGGGGTTCCT |
| Modified CK8 | CTAGACTAGCATGCTGCCCATGTAAGGAGGCAAGGCCTGGGGA |
| promoter | CACCCGAGATGCCTGGTTATAATTAACCCAGACATGTGGCTGCC |
| (SEQ ID NO: 100) | CCCCCCCCCCCAACACCTGCTGCCTCTAAAAATAACCCTGCATG |
| | CCATGTTCCCGGCGAAGGGCCAGCTGTCCCCCGCCAGCTAGAC |
| | TCAGCACTTAGTTTAGGAACCAGTGAGCAAGTCAGCCCTTGGG |
| | GCAGCCCATACAAGGCCATGGGGCTGGGCAAGCTGCACGCCTG |
| | GGTCCGGGGTGGGCACGGTGCCCGGGCAACGAGCTGAAAGCTC |
| | ATCTGCTCTCAGGGGCCCCTCCCTGGGGACAGCCCCTCCTGGCT |
| | AGTCACACCCTGTAGGCTCCTCTATATAACCCAGGGGCACAGG |
| | GGCTGCCCTCATTCTACCACCACCTCCACAGCACAGACAGACA |
| | CTCAGGAGCCAGCCAGCCAggtaagtttagtctttttgtcttttatttcaggtcccggatccg |
| | gtggtggtgcaaatcaaagaactgctcctcagtggatgttgcctttacttctaggcctgtacggaagtgttacg |
| | ccaCC |
| Minimal CK8 | CTAGACTAGCATGCTGCCCATGTAAGGAGGCAAGGCCTGGGGA |
| promoter | CACCCGAGATGCCTGGTTATAATTAACCCAGACATGTGGCTGCC |
| (SEQ ID NO: 101) | CCCCCCCCCCCAACACCTGCTGCCTCTAAAAATAACCCTGCATG |
| | CCATGTTCCCGGCGAAGGGCCAGCTGTCCCCCGCCAGCTAGAC |
| | TCAGCACTTAGTTTAGGAACCAGTGAGCAAGTCAGCCCTTGGG |
| | GCAGCCCATACAAGGCCATGGGGCTGGGCAAGCTGCACGCCTG |
| | GGTCCGGGGTGGGCACGGTGCCCGGGCAACGAGCTGAAAGCTC |
| | ATCTGCTCTCAGGGGCCCCTCCCTGGGGACAGCCCCTCCTGGCT |
| | AGTCACACCCTGTAGGCTCCTCTATATAACCCAGGGGCACAGG |
| | GGCTGCCCTCATTCTACCACCACCTCCACAGCACAGACAGACA |
| | CTCAGGAGCCAGCCAGCCAGCCACC |
| Full CK8 promoter | CTAGACTAGCATGCTGCCCATGTAAGGAGGCAAGGCCTGGGGA |
| (SEQ ID NO: 102) | CACCCGAGATGCCTGGTTATAATTAACCCAGACATGTGGCTGCC |
| | CCCCCCCCCCCAACACCTGCTGCCTCTAAAAATAACCCTGCATG |
| | CCATGTTCCCGGCGAAGGGCCAGCTGTCCCCCGCCAGCTAGAC |
| | TCAGCACTTAGTTTAGGAACCAGTGAGCAAGTCAGCCCTTGGG |
| | GCAGCCCATACAAGGCCATGGGGCTGGGCAAGCTGCACGCCTG |
| | GGTCCGGGGTGGGCACGGTGCCCGGGCAACGAGCTGAAAGCTC |
| | ATCTGCTCTCAGGGGCCCCTCCCTGGGGACAGCCCCTCCTGGCT |
| | AGTCACACCCTGTAGGCTCCTCTATATAACCCAGGGGCACAGG |
| | GGCTGCCCTCATTCTACCACCACCTCCACAGCACAGACAGACA |
| | CTCAGGAGCCAGCCAGCCATGGtctagaggatccggtactcgaggaactgaaaaac |
| | cagaaagttaactggtaagtttagtctttttgtcttttatttcaggtcccggatccggtggtggtgcaaatcaa |
| | agaactgctcctcagtggatgttgcctttacttctaggcctgtacggaagtgttacgccaCC |
| hU6 promoter | GGTGTTTCGTCCTTTCCACAAGATATATAAAGCCAAGAAATCG |
| (SEQ ID NO: 103) | AAATACTTTCAAGTTACGGTAAGCATATGATAGTCCATTTTAA |
| | AACATAATTTTAAAACTGCAAACTACCCAAGAAATTATTACTT |
| | TCTACGTCACGTATTTTGTACTAATATCTTTGTGTTTACAGTCA |
| | AATTAATTCCAATTATCTCTCTAACAGCCTTGTATCGTATATGC |
| | AAATATGAAGGAATCATGGGAAATAGGCCCTC |
| mU6 promoter | AAACAAGGCTTTTCTCCAAGGGATATTTATAGTCTCAAAACACA |
| (SEQ ID NO: 104) | CAATTACTTTACAGTTAGGGTGAGTTTCCTTTTGTGCTGTTTTTT |
| | AAAATAATAATTTAGTATTTGTATCTCTTATAGAAATCCAAGCC |
| | TATCATGTAAAATGTAGCTAGTATTAAAAAGAACAGATTATCT |
| | GTCTTTTATCGCACATTAAGCCTCTATAGTTACTAGGAAATATT |
| | ATATGCAAATTAACCGGGGCAGGGGAGTAGCCGAGCTTCTCCC |
| | ACAAGTCTGTGCGAGGGGGCCGGCGCGGGCCTAGAGATGGCG |
| | GCGTCGGATC |
| 7SK promoter | GaGGtaCcCAAGCGGCGCACAAGCtatataaacctgaaggaagtctcaactttacactta |
| (SEQ ID NO: 105) | ggtcaagttgcttatcgtactagagcttcagcaggaaatttaactaaaatctaatttaaccagcatagcaaata |
| | tcatttattcccaaaatgctaaagtttgagataaacggacttgatttccggctgttttgacactatccagaatg |
| | ccttgcaGATGGGTGGGGCATGCTAAATACTGCAG |
| H1 promoter | gggaaagaGTGGTCTCATACAGAACTTATAagattcccaaatccaaagacatttca |
| (SEQ ID NO: 106) | cgtttatggtgatttcccagaacacatagcgacatgcaaatattgcagggcgccactcccctgtccctcac |
| | agccatcttcctgccagggcgcacgcgcgctgggtgttcccgcctagtgacactgggcccgcgattcct |
| | tggagcgGGTTGATGACGTCAGCGTT |
| SaCas9 | ATGGCCCCAAAGAAGAAGCGGAAGGTCGGTATCCACGGAGTC |
| (SEQ ID NO: 107) | CCAGCAGCCAAGCGGAACTACATCCTGGGCCTGGACATCGGC |
| | ATCACCAGCGTGGGCTACGGCATCATCGACTACGAGACACGG |
| | GACGTGATCGATGCCGGCGTGCGGCTGTTCAAAGAGGCCAAC |
| | GTGGAAAACAACGAGGGCAGGCGGAGCAAGAGAGGCGCCAG |
| | AAGGCTGAAGCGGCGGAGGCGGCATAGAATCCAGAGAGTGAA |
| | GAAGCTGCTGTTCGACTACAACCTGCTGACCGACCACAGCGAG |
| | CTGAGCGGCATCAACCCCTACGAGGCCAGAGTGAAGGGCCTG |
| | AGCCAGAAGCTGAGCGAGGAAGAGTTCTCTGCCGCCCTGCTG |
| | CACCTGGCCAAGAGAAGAGGCGTGCACAACGTGAACGAGGTG |
| | GAAGAGGACACCGGCAACGAGCTGTCCACCAAAGAGCAGATC |
| | AGCCGGAACAGCAAGGCCCTGGAAGAGAAATACGTGGCCGAA |
| | CTGCAGCTGGAACGGCTGAAGAAAGACGGCGAAGTGCGGGGC |
| | AGCATCAACAGATTCAAGACCAGCGACTACGTGAAAGAAGCC |
| | AAACAGCTGCTGAAGGTGCAGAAGGCCTACCACCAGCTGGAC |
| | CAGAGCTTCATCGACACCTACATCGACCTGCTGGAAACCCGGC |
| | GGACCTACTATGAGGGACCTGGCGAGGGCAGCCCCTTCGGCT |
| | GGAAGGACATCAAAGAATGGTACGAGATGCTGATGGGCCACT |
| | GCACCTACTTCCCCGAGGAACTGCGGAGCGTGAAGTACGCCTA |
| | CAACGCCGACCTGTACAACGCCCTGAACGACCTGAACAATCTC |
| | GTGATCACCAGGGACGAGAACGAGAAGCTGGAATATTACGAG |
| | AAGTTCCAGATCATCGAGAACGTGTTCAAGCAGAAGAAGAAG |
| | CCCACCCTGAAGCAGATCGCCAAAGAAATCCTCGTGAACGAA |
| | GAGGATATTAAGGGCTACAGAGTGACCAGCACCGGCAAGCCC |
| | GAGTTCACCAACCTGAAGGTGTACCACGACATCAAGGACATT |
| | ACCGCCCGGAAAGAGATTATTGAGAACGCCGAGCTGCTGGAT |
| | CAGATTGCCAAGATCCTGACCATCTACCAGAGCAGCGAGGAC |
| | ATCCAGGAAGAACTGACCAATCTGAACTCCGAGCTGACCCAG |
| | GAAGAGATCGAGCAGATCTCTAATCTGAAGGGCTATACCGGC |
| | ACCCACAACCTGAGCCTGAAGGCCATCAACCTGATCCTGGACG |
| | AGCTGTGGCACACCAACGACAACCAGATCGCTATCTTCAACCG |
| | GCTGAAGCTGGTGCCCAAGAAGGTGGACCTGTCCCAGCAGAA |
| | AGAGATCCCCACCACCCTGGTGGACGACTTCATCCTGAGCCCC |
| | GTCGTGAAGAGAAGCTTCATCCAGAGCATCAAAGTGATCAAC |
| | GCCATCATCAAGAAGTACGGCCTGCCCAACGACATCATTATCG |
| | AGCTGGCCCGCGAGAAGAACTCCAAGGACGCCCAGAAAATGA |
| | TCAACGAGATGCAGAAGCGGAACCGGCAGACCAACGAGCGG |
| | ATCGAGGAAATCATCCGGACCACCGGCAAAGAGAACGCCAAG |
| | TACCTGATCGAGAAGATCAAGCTGCACGACATGCAGGAAGGC |
| | AAGTGCCTGTACAGCCTGGAAGCCATCCCTCTGGAAGATCTGC |
| | TGAACAACCCCTTCAACTATGAGGTGGACCACATCATCCCCAG |
| | AAGCGTGTCCTTCGACAACAGCTTCAACAACAAGGTGCTCGTG |
| | AAGCAGGAAGAAAACAGCAAGAAGGGCAACCGGACCCCATT |
| | CCAGTACCTGAGCAGCAGCGACAGCAAGATCAGCTACGAAAC |
| | CTTCAAGAAGCACATCCTGAATCTGGCCAAGGGCAAGGGCAG |
| | AATCAGCAAGACCAAGAAAGAGTATCTGCTGGAAGAACGGGA |
| | CATCAACAGGTTCTCCGTGCAGAAAGACTTCATCAACCGGAAC |
| | CTGGTGGATACCAGATACGCCACCAGAGGCCTGATGAACCTG |
| | CTGCGGAGCTACTTCAGAGTGAACAACCTGGACGTGAAAGTG |
| | AAGTCCATCAATGGCGGCTTCACCAGCTTTCTGCGGCGGAAGT |
| | GGAAGTTTAAGAAAGAGCGGAACAAGGGGTACAAGCACCACG |
| | CCGAGGACGCCCTGATCATTGCCAACGCCGATTTCATCTTCAA |
| | AGAGTGGAAGAAACTGGACAAGGCCAAAAAAGTGATGGAAA |
| | ACCAGATGTTCGAGGAAAAGCAGGCCGAGAGCATGCCCGAGA |
| | TCGAAACCGAGCAGGAGTACAAAGAGATCTTCATCACCCCCC |
| | ACCAGATCAAGCACATTAAGGACTTCAAGGACTACAAGTACA |
| | GCCACCGGGTGGACAAGAAGCCTAATAGAGAGCTGATTAACG |
| | ACACCCTGTACTCCACCCGGAAGGACGACAAGGGCAACACCC |
| | TGATCGTGAACAATCTGAACGGCCTGTACGACAAGGACAATG |
| | ACAAGCTGAAAAAGCTGATCAACAAGAGCCCCGAAAAGCTGC |
| | TGATGTACCACCACGACCCCCAGACCTACCAGAAACTGAAGCT |
| | GATTATGGAACAGTACGGCGACGAGAAGAATCCCCTGTACAA |
| | GTACTACGAGGAAACCGGGAACTACCTGACCAAGTACTCCAA |
| | AAAGGACAACGGCCCCGTGATCAAGAAGATTAAGTATTACGG |
| | CAACAAACTGAACGCCCATCTGGACATCACCGACGACTACCCC |
| | AACAGCAGAAACAAGGTCGTGAAGCTGTCCCTGAAGCCCTAC |
| | AGATTCGACGTGTACCTGGACAATGGCGTGTACAAGTTCGTGA |
| | CCGTGAAGAATCTGGATGTGATCAAAAAAGAAAACTACTACG |
| | AAGTGAATAGCAAGTGCTATGAGGAAGCTAAGAAGCTGAAGA |
| | AGATCAGCAACCAGGCCGAGTTTATCGCCTCCTTCTACAACAA |
| | CGATCTGATCAAGATCAACGGCGAGCTGTATAGAGTGATCGG |
| | CGTGAACAACGACCTGCTGAACCGGATCGAAGTGAACATGAT |
| | CGACATCACCTACCGCGAGTACCTGGAAAACATGAACGACAA |
| | GAGGCCCCCCAGGATCATTAAGACAATCGCCTCCAAGACCCA |
| | GAGCATTAAGAAGTACAGCACAGACATTCTGGGCAACCTGTA |
| | TGAAGTGAAATCTAAGAAGCACCCTCAGATCATCAAAAAGGG |
| | CAAAAGGCCGGCGGCCACGAAAAAGGCCGGCCAGGCAAAAA |
| | AGAAAAAGggatccGAATTCtag |
| Myospreader Cas9 | ATGCCCAAAAAGAAAAGAGCCGTGGAAGGTATCCACGGAGTC |
| (SEQ ID NO: 108) | CCAGCAGCCAAGCGGAACTACATCCTGGGCCTGGACATCGGC |
| | ATCACCAGCGTGGGCTACGGCATCATCGACTACGAGACACGG |
| | GACGTGATCGATGCCGGCGTGCGGCTGTTCAAAGAGGCCAAC |
| | GTGGAAAACAACGAGGGCAGGCGGAGCAAGAGAGGCGCCAG |
| | AAGGCTGAAGCGGCGGAGGCGGCATAGAATCCAGAGAGTGAA |
| | GAAGCTGCTGTTCGACTACAACCTGCTGACCGACCACAGCGAG |
| | CTGAGCGGCATCAACCCCTACGAGGCCAGAGTGAAGGGCCTG |
| | AGCCAGAAGCTGAGCGAGGAAGAGTTCTCTGCCGCCCTGCTG |
| | CACCTGGCCAAGAGAAGAGGCGTGCACAACGTGAACGAGGTG |
| | GAAGAGGACACCGGCAACGAGCTGTCCACCAAAGAGCAGATC |
| | AGCCGGAACAGCAAGGCCCTGGAAGAGAAATACGTGGCCGAA |
| | CTGCAGCTGGAACGGCTGAAGAAAGACGGCGAAGTGCGGGGC |
| | AGCATCAACAGATTCAAGACCAGCGACTACGTGAAAGAAGCC |
| | AAACAGCTGCTGAAGGTGCAGAAGGCCTACCACCAGCTGGAC |
| | CAGAGCTTCATCGACACCTACATCGACCTGCTGGAAACCCGGC |
| | GGACCTACTATGAGGGACCTGGCGAGGGCAGCCCCTTCGGCT |
| | GGAAGGACATCAAAGAATGGTACGAGATGCTGATGGGCCACT |
| | GCACCTACTTCCCCGAGGAACTGCGGAGCGTGAAGTACGCCTA |
| | CAACGCCGACCTGTACAACGCCCTGAACGACCTGAACAATCTC |
| | GTGATCACCAGGGACGAGAACGAGAAGCTGGAATATTACGAG |
| | AAGTTCCAGATCATCGAGAACGTGTTCAAGCAGAAGAAGAAG |
| | CCCACCCTGAAGCAGATCGCCAAAGAAATCCTCGTGAACGAA |
| | GAGGATATTAAGGGCTACAGAGTGACCAGCACCGGCAAGCCC |
| | GAGTTCACCAACCTGAAGGTGTACCACGACATCAAGGACATT |
| | ACCGCCCGGAAAGAGATTATTGAGAACGCCGAGCTGCTGGAT |
| | CAGATTGCCAAGATCCTGACCATCTACCAGAGCAGCGAGGAC |
| | ATCCAGGAAGAACTGACCAATCTGAACTCCGAGCTGACCCAG |
| | GAAGAGATCGAGCAGATCTCTAATCTGAAGGGCTATACCGGC |
| | ACCCACAACCTGAGCCTGAAGGCCATCAACCTGATCCTGGACG |
| | AGCTGTGGCACACCAACGACAACCAGATCGCTATCTTCAACCG |
| | GCTGAAGCTGGTGCCCAAGAAGGTGGACCTGTCCCAGCAGAA |
| | AGAGATCCCCACCACCCTGGTGGACGACTTCATCCTGAGCCCC |
| | GTCGTGAAGAGAAGCTTCATCCAGAGCATCAAAGTGATCAAC |
| | GCCATCATCAAGAAGTACGGCCTGCCCAACGACATCATTATCG |
| | AGCTGGCCCGCGAGAAGAACTCCAAGGACGCCCAGAAAATGA |
| | TCAACGAGATGCAGAAGCGGAACCGGCAGACCAACGAGCGG |
| | ATCGAGGAAATCATCCGGACCACCGGCAAAGAGAACGCCAAG |
| | TACCTGATCGAGAAGATCAAGCTGCACGACATGCAGGAAGGC |
| | AAGTGCCTGTACAGCCTGGAAGCCATCCCTCTGGAAGATCTGC |
| | TGAACAACCCCTTCAACTATGAGGTGGACCACATCATCCCCAG |
| | AAGCGTGTCCTTCGACAACAGCTTCAACAACAAGGTGCTCGTG |
| | AAGCAGGAAGAAAACAGCAAGAAGGGCAACCGGACCCCATT |
| | CCAGTACCTGAGCAGCAGCGACAGCAAGATCAGCTACGAAAC |
| | CTTCAAGAAGCACATCCTGAATCTGGCCAAGGGCAAGGGCAG |
| | AATCAGCAAGACCAAGAAAGAGTATCTGCTGGAAGAACGGGA |
| | CATCAACAGGTTCTCCGTGCAGAAAGACTTCATCAACCGGAAC |
| | CTGGTGGATACCAGATACGCCACCAGAGGCCTGATGAACCTG |
| | CTGCGGAGCTACTTCAGAGTGAACAACCTGGACGTGAAAGTG |
| | AAGTCCATCAATGGCGGCTTCACCAGCTTTCTGCGGCGGAAGT |
| | GGAAGTTTAAGAAAGAGCGGAACAAGGGGTACAAGCACCACG |
| | CCGAGGACGCCCTGATCATTGCCAACGCCGATTTCATCTTCAA |
| | AGAGTGGAAGAAACTGGACAAGGCCAAAAAAGTGATGGAAA |
| | ACCAGATGTTCGAGGAAAAGCAGGCCGAGAGCATGCCCGAGA |
| | TCGAAACCGAGCAGGAGTACAAAGAGATCTTCATCACCCCCC |
| | ACCAGATCAAGCACATTAAGGACTTCAAGGACTACAAGTACA |
| | GCCACCGGGTGGACAAGAAGCCTAATAGAGAGCTGATTAACG |
| | ACACCCTGTACTCCACCCGGAAGGACGACAAGGGCAACACCC |
| | TGATCGTGAACAATCTGAACGGCCTGTACGACAAGGACAATG |
| | ACAAGCTGAAAAAGCTGATCAACAAGAGCCCCGAAAAGCTGC |
| | TGATGTACCACCACGACCCCCAGACCTACCAGAAACTGAAGCT |
| | GATTATGGAACAGTACGGCGACGAGAAGAATCCCCTGTACAA |
| | GTACTACGAGGAAACCGGGAACTACCTGACCAAGTACTCCAA |
| | AAAGGACAACGGCCCCGTGATCAAGAAGATTAAGTATTACGG |
| | CAACAAACTGAACGCCCATCTGGACATCACCGACGACTACCCC |
| | AACAGCAGAAACAAGGTCGTGAAGCTGTCCCTGAAGCCCTAC |
| | AGATTCGACGTGTACCTGGACAATGGCGTGTACAAGTTCGTGA |
| | CCGTGAAGAATCTGGATGTGATCAAAAAAGAAAACTACTACG |
| | AAGTGAATAGCAAGTGCTATGAGGAAGCTAAGAAGCTGAAGA |
| | AGATCAGCAACCAGGCCGAGTTTATCGCCTCCTTCTACAACAA |
| | CGATCTGATCAAGATCAACGGCGAGCTGTATAGAGTGATCGG |
| | CGTGAACAACGACCTGCTGAACCGGATCGAAGTGAACATGAT |
| | CGACATCACCTACCGCGAGTACCTGGAAAACATGAACGACAA |
| | GAGGCCCCCCAGGATCATTAAGACAATCGCCTCCAAGACCCA |
| | GAGCATTAAGAAGTACAGCACAGACATTCTGGGCAACCTGTA |
| | TGAAGTGAAATCTAAGAAGCACCCTCAGATCATCAAAAAGGG |
| | CGCTCCTCCGGCACAACCTCCCAGCCAACCTCAGCAGCATTAT |
| | AGCGAAGGAGAGTTGGAGGAAGATGAAGATTCTGATGACGCG |
| | CGCAAAAAACGCAAAAAAAAAtag |
| EFS promoter | TCGAGTGGCTCCGGTGCCCGTCAGTGGGCAGAGCGCACAT |
| (SEQ ID NO: 121) | CGCCCACAGTCCCCGAGAAGTTGGGGGGAGGGGTCGGCA |
| | ATTGAACCGGTGCCTAGAGAAGGTGGCGCGGGGTAAACTG |
| | GGAAAGTGATGTCGTGTACTGGCTCCGCCTTTTTCCCGAGGGTG |
| | GGGGAGAACCGTATATAAGTGCAGTAGTCGCCGTGAACGTTCTT |
| | TTTCGCAACGGGTTTGCCGCCAGAACACAGGTGTCGTGACC |
| | GCGG |
| Mini | TAGCAATAAAGGATCGTTTATTTTCATTGGAAGCGTGTGTTGGTT |
| polyadenylation | TTTTGATCAGGCGCG |
| signal | |
| (SEQ ID NO: 122) | |
| SaCas9 guide | TCTCGCCAACAAGTTGACGAGATAAACACGGCATTTTGCCTT |
| RNA scaffold | GTTTTAGTAGATTCTGTTTCCAGAGTACTAAAAC |
| (SEQ ID NO: 123) | |

TABLE 7
Experimental design for treatment of Δ52/mdx mice with gene editing constructs

| | | Age at | In-life | Dose | Admin |
|---|---|---|---|---|---|
| Test/Control Article | Strain | treatment | duration | (vg/kg) | route |
| Saline | huDMD | 4-6 wks | 12 wks | N/A | IV |
| Saline | huDMDdel52 | 4-6 wks | 12 wks | | IV |
| AAVrh74.pAAV30 | huDMDdel52 | 4-6 wks | 12 wks | 2.5 × 10¹⁴ vg | IV |
| AAVrh74.pAAV93 | huDMDdel52 | 4-6 wks | 12 wks | 2.5 × 10¹⁴ vg | IV |
| AAVrh74.pAAV94 | huDMDdel52 | 4-6 wks | 12 wks | 2.5 × 10¹⁴ vg | IV |
| AAVrh74.pAAV95 | huDMDdel52 | 4-6 wks | 12 wks | 2.5 × 10¹⁴ vg | IV |
| AAVrh74.pAAV96 | huDMDdel52 | 4-6 wks | 12 wks | 2.5 × 10¹⁴ vg | IV |
| AAVrh74.pAAV97 | huDMDdel52 | 4-6 wks | 12 wks | 2.5 × 10¹⁴ vg | IV |
| AAVrh74.pAAV99 | huDMDdel52 | 4-6 wks | 12 wks | 2.5 × 10¹⁴ vg | IV |
| AAVrh74.pAAV102 | huDMDdel52 | 4-6 wks | 12 wks | 2.5 × 10¹⁴ vg | IV |

TABLE 8
Immunofluorescence conditions

| Antibody/Stain | Catalog No. | Clone | Dilution |
|---|---|---|---|
| Dystrophin | Santa Cruz SC-33697 | 4C7 | 1:50 |
| Laminin | Sigma-Aldrich L9393 | Polyclonal | 1:50 |
| SaCas9 | AbCam ab203943 | EPR19795 | 1:100 |
| DAPI | D1306 | N/A | 0.05 μg/mL |

1.-67. (canceled)

68. A genetic construct, comprising:
i) a first inverted terminal repeat (ITR) nucleotide sequence;
ii) an RNA polymerase II (Pol II)-driven promoter operably linked to a transgene;
iii) a first RNA polymerase III (Pol III)-driven promoter operably linked to a first guide RNA (gRNA) nucleotide sequence as set forth in SEQ ID NO: 14;
iv) a second Poll III-driven promoter operably linked to a second gRNA nucleotide sequence as set forth in SEQ ID NO: 31,
wherein the first and second Pol III-driven promoters and the first and second gRNAs are in a reverse orientation to the Pol II-driven promoter and the transgene; and
v) a second ITR nucleotide sequence,
wherein said genetic construct is encoded in a single polynucleotide.

69. The genetic construct of claim 68, wherein the first ITR nucleotide sequence and the second ITR nucleotide sequence are set forth in SEQ ID NO: 99.

70. The genetic construct of claim 68, wherein the Pol II promoter is a modified CK8 promoter comprising the nucleotide sequence set forth in SEQ ID NO: 100.

71. The genetic construct of claim 68, wherein the Pol II promoter is a minimal CK8 promoter comprising the nucleotide sequence set forth in SEQ ID NO: 101.

72. The genetic construct of claim 68, wherein the transgene is a Cas9 nucleotide sequence, as set forth in SEQ ID NO: 107.

73. The genetic construct of claim 68, wherein the transgene is a Cas9 nucleotide sequence, as set forth in SEQ ID NO: 108.

74. The genetic construct of claim 68, wherein the first Pol III-driven promoter and the second Pol III-driven promoter are human U6 (hU6) promoters, comprising the nucleotide sequence set forth in SEQ ID NO: 103.

75. The genetic construct of claim 68, wherein the single polynucleotide is at least 99% identical to SEQ ID NO: 140.

76. The genetic construct of claim 75, wherein the single polynucleotide is identical to SEQ ID NO: 140.

77. A vector capable of expressing the genetic construct of claim 68, wherein the vector is an adeno-associated virus (AAV) vector.

78. The AAV vector of claim 77, wherein the vector is a rh.74 AAV vector or a recombinant variant thereof.

79. The AAV vector of claim 77, wherein the vector is a MyoAAV-4E vector or a recombinant variant thereof.

80. A eukaryotic cell comprising the genetic construct of claim 68.

81. A kit comprising the genetic construct of claim 68.

82. A method of treating a subject having a mutant gene, the method comprising administering to the subject the genetic construct of claim 68.

83. A method of treating a disease in a patient in need thereof, the method comprising administering to the patient the genetic construct of claim 68.

84. The method of claim 83, wherein the disease is Duchenne muscular dystrophy.

85. The method of claim 83, wherein the disease is Becker muscular dystrophy.

86. The method of claim 83, wherein the genetic construct is administered to the patient intramuscularly, intravenously, or a combination thereof.


SEQUENCE LISTING (143 sequences)

SEQ ID NO: 1 - RNA, 21 nt, Synthetic construct
  source 1..21: mol_type = other RNA
caaaaggagccaggctagaag

SEQ ID NO: 2 - RNA, 21 nt, Synthetic construct
  source 1..21: mol_type = other RNA
gcattctcacactataaatga

SEQ ID NO: 3 - RNA, 21 nt, Synthetic construct
  source 1..21: mol_type = other RNA
atcattgataattggtagaca

SEQ ID NO: 4 - RNA, 21 nt, Synthetic construct
  source 1..21: mol_type = other RNA
caggtattcttaacatggatg

SEQ ID NO: 5 - RNA, 21 nt, Synthetic construct
  source 1..21: mol_type = other RNA
gtttactcagtacccacctga

SEQ ID NO: 6 - RNA, 21 nt, Synthetic construct
  source 1..21: mol_type = other RNA
atgatcattgagactacccac

SEQ ID NO: 7 - RNA, 21 nt, Synthetic construct
  source 1..21: mol_type = other RNA
ggtatccttgagtcttactgt

SEQ ID NO: 8 - RNA, 21 nt, Synthetic construct
  source 1..21: mol_type = other RNA
agtaacctcatagattgtggt

SEQ ID NO: 9 - RNA, 21 nt, Synthetic construct
  source 1..21: mol_type = other RNA
cttgtacaaaaatgatgagtg

SEQ ID NO: 10 - RNA, 21 nt, Synthetic construct
  source 1..21: mol_type = other RNA
ggatctggcctaggacaaatg

SEQ ID NO: 11 - RNA, 21 nt, Synthetic construct
  source 1..21: mol_type = other RNA
cctagctgttgcacccaagag

SEQ ID NO: 12 - RNA, 21 nt, Synthetic construct
  source 1..21: mol_type = other RNA
agccctttgaagatcatagcc

SEQ ID NO: 13 - RNA, 21 nt, Synthetic construct
  source 1..21: mol_type = other RNA
aggggcacaggacagatgcca

SEQ ID NO: 14 - RNA, 21 nt, Synthetic construct
  source 1..21: mol_type = other RNA
gtcagtgctgaagcaaatgtg

SEQ ID NO: 15 - RNA, 21 nt, Synthetic construct
  source 1..21: mol_type = other RNA
cacatttaaagagctaagcct

SEQ ID NO: 16 - RNA, 21 nt, Synthetic construct
  source 1..21: mol_type = other RNA
cagggataataaaagaaggga

SEQ ID NO: 17 - RNA, 21 nt, Synthetic construct
  source 1..21: mol_type = other RNA
actgtgggggcacaattagga

SEQ ID NO: 18 - RNA, 21 nt, Synthetic construct
  source 1..21: mol_type = other RNA
aatacaaagctaggtttaggg

SEQ ID NO: 19 - RNA, 21 nt, Synthetic construct
  source 1..21: mol_type = other RNA
gcccagtttagcaaacaggat

SEQ ID NO: 20 - RNA, 21 nt, Synthetic construct
  source 1..21: mol_type = other RNA
caggttgtaagataaatgtcc

SEQ ID NO: 21 - RNA, 21 nt, Synthetic construct
  source 1..21: mol_type = other RNA
agttcacctcagggactgatg

SEQ ID NO: 22 - RNA, 21 nt, Synthetic construct
  source 1..21: mol_type = other RNA
catgaagggccatgtaagtag

SEQ ID NO: 23 - RNA, 21 nt, Synthetic construct
  source 1..21: mol_type = other RNA
ttcagtaaagagggataaggc

SEQ ID NO: 24 - RNA, 21 nt, Synthetic construct
  source 1..21: mol_type = other RNA
acagacatgggttacatgggt

SEQ ID NO: 25 - RNA, 21 nt, Synthetic construct
  source 1..21: mol_type = other RNA
ggccttcagtttatcaacaca

SEQ ID NO: 26 - RNA, 21 nt, Synthetic construct
  source 1..21: mol_type = other RNA
aatgggggccctgtactaggg

SEQ ID NO: 27 - RNA, 21 nt, Synthetic construct
  source 1..21: mol_type = other RNA
ctgttacatgtggccacaggg

SEQ ID NO: 28 - RNA, 21 nt, Synthetic construct
  source 1..21: mol_type = other RNA
tacaccaacctatatttgact

SEQ ID NO: 29 - RNA, 21 nt, Synthetic construct
  source 1..21: mol_type = other RNA
atgacttatcaaaggatatgc

SEQ ID NO: 30 - RNA, 21 nt, Synthetic construct
  source 1..21: mol_type = other RNA
tcctgaattgtagtaaaagag

SEQ ID NO: 31 - RNA, 21 nt, Synthetic construct
  source 1..21: mol_type = other RNA
gagtggtgttgacctttgtcg

SEQ ID NO: 32 - RNA, 21 nt, Synthetic construct
  source 1..21: mol_type = other RNA
cccttatctgagaaagaccag

SEQ ID NO: 33 - RNA, 21 nt, Synthetic construct
  source 1..21: mol_type = other RNA
gaacctcaaagagaggaggcc

SEQ ID NO: 34 - RNA, 21 nt, Synthetic construct
  source 1..21: mol_type = other RNA
gaaagtacataggaccttggt

SEQ ID NO: 35 - RNA, 21 nt, Synthetic construct
  source 1..21: mol_type = other RNA
tatccgtaatataagttgcaa

SEQ ID NO: 36 - RNA, 21 nt, Synthetic construct
  source 1..21: mol_type = other RNA
gtagccccctgcagtatctag

SEQ ID NO: 37 - RNA, 21 nt, Synthetic construct
  source 1..21: mol_type = other RNA
agtgtcatcagaatatgatgc

SEQ ID NO: 38 - RNA, 21 nt, Synthetic construct
  source 1..21: mol_type = other RNA
catggaccaagcccaaagtga

SEQ ID NO: 39 - RNA, 21 nt, Synthetic construct
  source 1..21: mol_type = other RNA
gtaagtcataatttcataagc

SEQ ID NO: 40 - RNA, 21 nt, Synthetic construct
  source 1..21: mol_type = other RNA
gcccttgcctattcaacaagc

SEQ ID NO: 41 - RNA, 21 nt, Synthetic construct
  source 1..21: mol_type = other RNA
gctgctacccaagttatcctg

SEQ ID NO: 42 - RNA, 21 nt, Synthetic construct
  source 1..21: mol_type = other RNA
ttctacccctgccttagaagg

SEQ ID NO: 43 - RNA, 21 nt, Synthetic construct
  source 1..21: mol_type = other RNA
tgagtccaatccatgatccag

SEQ ID NO: 44 - RNA, 21 nt, Synthetic construct
  source 1..21: mol_type = other RNA
tgatccagcagagtttcagcc

SEQ ID NO: 45 - RNA, 21 nt, Synthetic construct
  source 1..21: mol_type = other RNA
attgtaatagctagttagagg

SEQ ID NO: 46 - RNA, 21 nt, Synthetic construct
  source 1..21: mol_type = other RNA
cagacccaaacagtaagactc

SEQ ID NO: 47 - RNA, 21 nt, Synthetic construct
  source 1..21: mol_type = other RNA
agattgcatggatttaatgtg

SEQ ID NO: 48 - RNA, 21 nt, Synthetic construct
  source 1..21: mol_type = other RNA
ggttgtgcatactaattgcca

SEQ ID NO: 49 - RNA, 21 nt, Synthetic construct
  source 1..21: mol_type = other RNA
gaagccttagtaagatcagag

SEQ ID NO: 50 - RNA, 21 nt, Synthetic construct
  source 1..21: mol_type = other RNA
tgtttgaagccttagtaagat

SEQ ID NO: 51 - RNA, 21 nt, Synthetic construct
  source 1..21: mol_type = other RNA
cttcgggttcagttgaaagca

SEQ ID NO: 52 - RNA, 21 nt, Synthetic construct
  source 1..21: mol_type = other RNA
gacagggatttgacaaaaatg

SEQ ID NO: 53 - RNA, 21 nt, Synthetic construct
  source 1..21: mol_type = other RNA
agagttagaacactagcaagg

SEQ ID NO: 54 - RNA, 21 nt, Synthetic construct
  source 1..21: mol_type = other RNA
gataagcagtggtgccaaggg

SEQ ID NO: 55 - RNA, 21 nt, Synthetic construct
  source 1..21: mol_type = other RNA
aggagataagcagtggtgcca

SEQ ID NO: 56 - RNA, 21 nt, Synthetic construct
  source 1..21: mol_type = other RNA
gggctgaaactctgctggatc

SEQ ID NO: 57 - RNA, 21 nt, Synthetic construct
  source 1..21: mol_type = other RNA
aaatcctgggctgaaactctg

SEQ ID NO: 58 - RNA, 21 nt, Synthetic construct
  source 1..21: mol_type = other RNA
aaggcagcagaatttagccac

SEQ ID NO: 59 - RNA, 21 nt, Synthetic construct
  source 1..21: mol_type = other RNA
cacatatatggcataaaagcc

SEQ ID NO: 60 - RNA, 21 nt, Synthetic construct
  source 1..21: mol_type = other RNA
agttgttattagagatgatgt

SEQ ID NO: 61 - RNA, 21 nt, Synthetic construct
  source 1..21: mol_type = other RNA
cctgcataaactttgagaggg

SEQ ID NO: 62 - RNA, 21 nt, Synthetic construct
  source 1..21: mol_type = other RNA
tgtctaccaattatcaatgat

SEQ ID NO: 63 - RNA, 21 nt, Synthetic construct
  source 1..21: mol_type = other RNA
agcatggtgtcagaaggaact

SEQ ID NO: 64 - RNA, 21 nt, Synthetic construct
  source 1..21: mol_type = other RNA
ctctcgatagaagctaaagtg

SEQ ID NO: 65 - RNA, 21 nt, Synthetic construct
  source 1..21: mol_type = other RNA
gataatgaaaaattgggacct

SEQ ID NO: 66 - RNA, 21 nt, Synthetic construct
  source 1..21: mol_type = other RNA
tagtgttaagttgaacgattg

SEQ ID NO: 67 - RNA, 21 nt, Synthetic construct
  source 1..21: mol_type = other RNA
gcacttctgtatgagaggctg

SEQ ID NO: 68 - RNA, 21 nt, Synthetic construct
  source 1..21: mol_type = other RNA
caggttgtaagataaatgtcc

SEQ ID NO: 69 - RNA, 21 nt, Synthetic construct
  source 1..21: mol_type = other RNA
agttcacctcagggactgatg

SEQ ID NO: 70 - RNA, 21 nt, Synthetic construct
  source 1..21: mol_type = other RNA
gcttcacaatgatcagggtgg

SEQ ID NO: 71 - RNA, 21 nt, Synthetic construct
  source 1..21: mol_type = other RNA
gcttcacaatgatcagggtgg

SEQ ID NO: 72 - RNA, 21 nt, Synthetic construct
  source 1..21: mol_type = other RNA
agggtggaatgaagagctctg

SEQ ID NO: 73 - RNA, 21 nt, Synthetic construct
  source 1..21: mol_type = other RNA
gataaggcttgagtggtcaca

SEQ ID NO: 74 - RNA, 21 nt, Synthetic construct
  source 1..21: mol_type = other RNA
cagagttttaaaagatgtgct

SEQ ID NO: 75 - RNA, 21 nt, Synthetic construct
  source 1..21: mol_type = other RNA
tgacaagcaagttcatttgtt

SEQ ID NO: 76 - RNA, 21 nt, Synthetic construct
  source 1..21: mol_type = other RNA
aagcccaagccccgccaacag

SEQ ID NO: 77 - RNA, 21 nt, Synthetic construct
  source 1..21: mol_type = other RNA
taagagtagcagagcatttag

SEQ ID NO: 78 - RNA, 21 nt, Synthetic construct
  source 1..21: mol_type = other RNA
gaagttagatgaagaaagacg

SEQ ID NO: 79 - RNA, 21 nt, Synthetic construct
  source 1..21: mol_type = other RNA
cctatatttgactcagggttg

SEQ ID NO: 80 - RNA, 21 nt, Synthetic construct
  source 1..21: mol_type = other RNA
aacaatatcttgacaccacca

SEQ ID NO: 81 - RNA, 21 nt, Synthetic construct
  source 1..21: mol_type = other RNA
aaacggacaggtgaaagctga

SEQ ID NO: 82 - RNA, 21 nt, Synthetic construct
  source 1..21: mol_type = other RNA
tgggtgggtaaccatggagaa

SEQ ID NO: 83 - RNA, 21 nt, Synthetic construct
  source 1..21: mol_type = other RNA
ttctggcttagagaagaagat

SEQ ID NO: 84 - RNA, 21 nt, Synthetic construct
  source 1..21: mol_type = other RNA
tttgatccacccttctaaggc

SEQ ID NO: 85 - RNA, 21 nt, Synthetic construct
  source 1..21: mol_type = other RNA
acagtgtagccaatgagagct

SEQ ID NO: 86 - RNA, 21 nt, Synthetic construct
  source 1..21: mol_type = other RNA
tcctcagagactattatcctg

SEQ ID NO: 87 - RNA, 21 nt, Synthetic construct
  source 1..21: mol_type = other RNA
tttggtttatgagcagggaca

SEQ ID NO: 88 - RNA, 21 nt, Synthetic construct
  source 1..21: mol_type = other RNA
tgttaccttggattttcaaag

SEQ ID NO: 89 - RNA, 21 nt, Synthetic construct
  source 1..21: mol_type = other RNA
gttggttttattatgaggtgg

SEQ ID NO: 90 - RNA, 21 nt, Synthetic construct
  source 1..21: mol_type = other RNA
attgaagccttgacataactt

SEQ ID NO: 91 - RNA, 21 nt, Synthetic construct
  source 1..21: mol_type = other RNA
ataggtatcacctatggatgc

SEQ ID NO: 92 - RNA, 21 nt, Synthetic construct
  source 1..21: mol_type = other RNA
cagagttaaaatactgacttg

SEQ ID NO: 93 - RNA, 21 nt, Synthetic construct
  source 1..21: mol_type = other RNA
ttctcatgtaaacagtctaag

SEQ ID NO: 94 - RNA, 21 nt, Synthetic construct
  source 1..21: mol_type = other RNA
aaggtacttctgactttccaa

SEQ ID NO: 95 - RNA, 21 nt, Synthetic construct
  source 1..21: mol_type = other RNA
aaagggccatagtagcagaag

SEQ ID NO: 96 - RNA, 21 nt, Synthetic construct
  source 1..21: mol_type = other RNA
gcttttctacccctgccttag

SEQ ID NO: 97 - RNA, 21 nt, Synthetic construct
  source 1..21: mol_type = other RNA
cctttacacaagaagctacag

SEQ ID NO: 98 - RNA, 21 nt, Synthetic construct
  source 1..21: mol_type = other RNA
gtacttcacagagccaggggg

SEQ ID NO: 99 - DNA, 128 bp, Synthetic construct
  source 1..128: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 100 - DNA, 575 bp, Synthetic construct
  source 1..575: mol_type = other DNA
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacgccacc

SEQ ID NO: 101 - DNA, 458 bp, Synthetic construct
  source 1..458: mol_type = other DNA
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccacc

SEQ ID NO: 102 - DNA, 625 bp, Synthetic construct
  source 1..625: mol_type = other DNA
ctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccacc

SEQ ID NO: 103 - DNA, 249 bp, Synthetic construct
  source 1..249: mol_type = other DNA
ggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctc

SEQ ID NO: 104 - DNA, 315 bp, Synthetic construct
  source 1..315: mol_type = other DNA
aaacaaggcttttctccaagggatatttatagtctcaaaacacacaattactttacagttagggtgagtttccttttgtgctgttttttaaaataataatttagtatttgtatctcttatagaaatccaagcctatcatgtaaaatgtagctagtattaaaaagaacagattatctgtcttttatcgcacattaagcctctatagttactaggaaatattatatgcaaattaaccggggcaggggagtagccgagcttctcccacaagtctgtgcgagggggccggcgcgggcctagagatggcggcgtcggatc

SEQ ID NO: 105 - DNA, 243 bp, Synthetic construct
  source 1..243: mol_type = other DNA
gaggtacccaagcggcgcacaagctatataaacctgaaggaagtctcaactttacacttaggtcaagttgcttatcgtactagagcttcagcaggaaatttaactaaaatctaatttaaccagcatagcaaatatcatttattcccaaaatgctaaagtttgagataaacggacttgatttccggctgttttgacactatccagaatgccttgcagatgggtggggcatgctaaatactgcag

SEQ ID NO: 106 - DNA, 222 bp, Synthetic construct
  source 1..222: mol_type = other DNA
gggaaagagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtt

SEQ ID NO: 107 - DNA, 3270 bp, Synthetic construct
  source 1..3270: mol_type = other DNA
atggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctag

SEQ ID NO: 108 - DNA, 3315 bp, Synthetic construct
  source 1..3315: mol_type = other DNA
atgcccaaaaagaaaagagccgtggaaggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcgctcctccggcacaacctcccagccaacctcagcagcattatagcgaaggagagttggaggaagatgaagattctgatgacgcgcgcaaaaaacgcaaaaaaaaatag

SEQ ID NO: 109 - DNA, 8485 bp, Synthetic construct
  source 1..8485: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 110 - DNA, 8429 bp, Synthetic construct
  source 1..8429: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 111 - DNA, 8401 bp, Synthetic construct
  source 1..8401: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 112 - DNA, 8513 bp, Synthetic construct
  source 1..8513: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 113 - DNA, 8602 bp, Synthetic construct
  source 1..8602: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 114 - DNA, 8546 bp, Synthetic construct
  source 1..8546: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 115 - DNA, 8518 bp, Synthetic construct
  source 1..8518: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 116 - DNA, 8630 bp, Synthetic construct
  source 1..8630: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 117 - DNA, 8680 bp, Synthetic construct
  source 1..8680: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 118 - DNA, 8652 bp, Synthetic construct
  source 1..8652: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 119 - DNA, 8596 bp, Synthetic construct
  source 1..8596: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 120 - DNA, 8568 bp, Synthetic construct
  source 1..8568: mol_type = other DNA
tggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 121 - DNA, 252 bp, Synthetic construct
  source 1..252: mol_type = other DNA
tcgagtggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtgtcgtgaccgcgg

SEQ ID NO: 122 - DNA, 60 bp, Synthetic construct
  source 1..60: mol_type = other DNA
tagcaataaaggatcgtttattttcattggaagcgtgtgttggttttttgatcaggcgcg

SEQ ID NO: 123 - DNA, 76 bp, Synthetic construct
  source 1..76: mol_type = other DNA
tctcgccaacaagttgacgagataaacacggcattttgccttgttttagtagattctgtttccagagtactaaaac

SEQ ID NO: 124 - DNA, 8675 bp, Synthetic construct
  source 1..8675: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaactggcatctgtcctgtgcccctcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaactgtgttgataaactgaaggcccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 125 - DNA, 8675 bp, Synthetic construct
  source 1..8675: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaacctcttgggtgcaacagctaggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaacccctagtacagggcccccattcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 126 - DNA, 8675 bp, Synthetic construct
  source 1..8675: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaaccatccatgttaagaatacctgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaacccctgtggccacatgtaacagcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 127 - DNA, 8675 bp, Synthetic construct
  source 1..8675: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaaccacatttgcttcagcactgaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaaccgacaaaggtcaacaccactccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 128 - DNA, 8675 bp, Synthetic construct
  source 1..8675: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaacaccacaatctatgaggttactcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaacctggtctttctcagataagggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 129 - DNA, 8675 bp, Synthetic construct
  source 1..8675: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaactgtctaccaattatcaatgatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttgtcgaccaaaaaatctcgccaacaagttgacgagataaacacggcattttgccttgttttagtagattctgtttccagagtactaaaacaccaaggtcctatgtactttccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 130 - DNA, 8675 bp, Synthetic construct
  source 1..8675: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaaccacatttgcttcagcactgaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttgtcgaccaaaaaatctcgccaacaagttgacgagataaacacggcattttgccttgttttagtagattctgtttccagagtactaaaacttgcaacttatattacggatacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 131 - DNA, 8474 bp, Synthetic construct
  source 1..8474: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccaccatgcccaaaaagaaaagagccgtggaaggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcgctcctccggcacaacctcccagccaacctcagcagcattatagcgaaggagagttggaggaagatgaagattctgatgacgcgcgcaaaaaacgcaaaaaaaaatagcaataaaggatcgtttattttcattggaagcgtgtgttggttttttgatcaggcgcgggtaccaaaaatctcgccaacaagttgacgagataaacacggcattttgccttgttttagtagattctgtttccagagtactaaaacacatttcctctctatacaaatgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcctcgactagtagaaaaatctcgccaacaagttgacgagataaacacggcattttgccttgttttagtagattctgtttccagagtactaaaacgtgccaataatttcattactatatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 132 - DNA, 8444 bp, Synthetic construct
  source 1..8444: mol_type = other DNA
cggtgcgggcctcttcgctattacgccagctgcgcgctcgctcgctcactgaggccgcccgggcaaagcccgggcgtcgggcgacctttggtcgcccggcctcagtgagcgagcgagcgcgcagagagggagtggccaactccatcactaggggttcctctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccaccatgcccaaaaagaaaagagccgtggaaggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcgctcctccggcacaacctcccagccaacctcagcagcattatagcgaaggagagttggaggaagatgaagattctgatgacgcgcgcaaaaaacgcaaaaaaaaatagcaataaaggatcgtttattttcattggaagcgtgtgttggttttttgatcaggcgcgaaaaaatctcgccaacaagttgacgagataaacacggcattttgccttgttatagtagattctgtttccagagtactataaccacatttgcttcagcactgaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaaatctcgccaacaagttgacgagataaacacggcattttgccttgttatagtagattctgtttccagagtactataaccgacaaaggtcaacaccactccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgat

SEQ ID NO: 133 - DNA, 5023 bp, Synthetic construct
  source 1..5023: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaactggcatctgtcctgtgcccctcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaactgtgttgataaactgaaggcccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 134 - DNA, 5023 bp, Synthetic construct
  source 1..5023: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaacctcttgggtgcaacagctaggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaacccctagtacagggcccccattcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 135 - DNA, 5023 bp, Synthetic construct
  source 1..5023: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaaccatccatgttaagaatacctgcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaacccctgtggccacatgtaacagcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 136 - DNA, 5023 bp, Synthetic construct
  source 1..5023: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaaccacatttgcttcagcactgaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaaccgacaaaggtcaacaccactccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 137 - DNA, 5023 bp, Synthetic construct
  source 1..5023: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaacaccacaatctatgaggttactcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttcctaggcaaaaaatctcgccaacaagttgacgagataaacacggcattttgccttgttttagtagattctgtttccagagtactaaaacctggtctttctcagataagggcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 138 - DNA, 5023 bp, Synthetic construct
  source 1..5023: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaactgtctaccaattatcaatgatcggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttgtcgaccaaaaaatctcgccaacaagttgacgagataaacacggcattttgccttgttttagtagattctgtttccagagtactaaaacaccaaggtcctatgtactttccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 139 - DNA, 5023 bp, Synthetic construct
  source 1..5023: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagcggccgcctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccatggtctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacgccaccatggccccaaagaagaagcggaaggtcggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcaaaaggccggcggccacgaaaaaggccggccaggcaaaaaagaaaaagggatccgaattctagcaataaaggatcgtttattttcattggaagcgtgtgttggttttttgatcaggcgcgggtaccaaaaaatctcgccaacaagttgacgagataaacacggcattttgccttgttttagtagattctgtttccagagtactaaaaccacatttgcttcagcactgaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttgtcgaccaaaaaatctcgccaacaagttgacgagataaacacggcattttgccttgttttagtagattctgtttccagagtactaaaacttgcaacttatattacggatacggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcgctagctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 140 - DNA, 4792 bp, Synthetic construct
  source 1..4792: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttcctctagactagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagccagccaccatgcccaaaaagaaaagagccgtggaaggtatccacggagtcccagcagccaagcggaactacatcctgggcctggacatcggcatcaccagcgtgggctacggcatcatcgactacgagacacgggacgtgatcgatgccggcgtgcggctgttcaaagaggccaacgtggaaaacaacgagggcaggcggagcaagagaggcgccagaaggctgaagcggcggaggcggcatagaatccagagagtgaagaagctgctgttcgactacaacctgctgaccgaccacagcgagctgagcggcatcaacccctacgaggccagagtgaagggcctgagccagaagctgagcgaggaagagttctctgccgccctgctgcacctggccaagagaagaggcgtgcacaacgtgaacgaggtggaagaggacaccggcaacgagctgtccaccaaagagcagatcagccggaacagcaaggccctggaagagaaatacgtggccgaactgcagctggaacggctgaagaaagacggcgaagtgcggggcagcatcaacagattcaagaccagcgactacgtgaaagaagccaaacagctgctgaaggtgcagaaggcctaccaccagctggaccagagcttcatcgacacctacatcgacctgctggaaacccggcggacctactatgagggacctggcgagggcagccccttcggctggaaggacatcaaagaatggtacgagatgctgatgggccactgcacctacttccccgaggaactgcggagcgtgaagtacgcctacaacgccgacctgtacaacgccctgaacgacctgaacaatctcgtgatcaccagggacgagaacgagaagctggaatattacgagaagttccagatcatcgagaacgtgttcaagcagaagaagaagcccaccctgaagcagatcgccaaagaaatcctcgtgaacgaagaggatattaagggctacagagtgaccagcaccggcaagcccgagttcaccaacctgaaggtgtaccacgacatcaaggacattaccgcccggaaagagattattgagaacgccgagctgctggatcagattgccaagatcctgaccatctaccagagcagcgaggacatccaggaagaactgaccaatctgaactccgagctgacccaggaagagatcgagcagatctctaatctgaagggctataccggcacccacaacctgagcctgaaggccatcaacctgatcctggacgagctgtggcacaccaacgacaaccagatcgctatcttcaaccggctgaagctggtgcccaagaaggtggacctgtcccagcagaaagagatccccaccaccctggtggacgacttcatcctgagccccgtcgtgaagagaagcttcatccagagcatcaaagtgatcaacgccatcatcaagaagtacggcctgcccaacgacatcattatcgagctggcccgcgagaagaactccaaggacgcccagaaaatgatcaacgagatgcagaagcggaaccggcagaccaacgagcggatcgaggaaatcatccggaccaccggcaaagagaacgccaagtacctgatcgagaagatcaagctgcacgacatgcaggaaggcaagtgcctgtacagcctggaagccatccctctggaagatctgctgaacaaccccttcaactatgaggtggaccacatcatccccagaagcgtgtccttcgacaacagcttcaacaacaaggtgctcgtgaagcaggaagaaaacagcaagaagggcaaccggaccccattccagtacctgagcagcagcgacagcaagatcagctacgaaaccttcaagaagcacatcctgaatctggccaagggcaagggcagaatcagcaagaccaagaaagagtatctgctggaagaacgggacatcaacaggttctccgtgcagaaagacttcatcaaccggaacctggtggataccagatacgccaccagaggcctgatgaacctgctgcggagctacttcagagtgaacaacctggacgtgaaagtgaagtccatcaatggcggcttcaccagctttctgcggcggaagtggaagtttaagaaagagcggaacaaggggtacaagcaccacgccgaggacgccctgatcattgccaacgccgatttcatcttcaaagagtggaagaaactggacaaggccaaaaaagtgatggaaaaccagatgttcgaggaaaagcaggccgagagcatgcccgagatcgaaaccgagcaggagtacaaagagatcttcatcaccccccaccagatcaagcacattaaggacttcaaggactacaagtacagccaccgggtggacaagaagcctaatagagagctgattaacgacaccctgtactccacccggaaggacgacaagggcaacaccctgatcgtgaacaatctgaacggcctgtacgacaaggacaatgacaagctgaaaaagctgatcaacaagagccccgaaaagctgctgatgtaccaccacgacccccagacctaccagaaactgaagctgattatggaacagtacggcgacgagaagaatcccctgtacaagtactacgaggaaaccgggaactacctgaccaagtactccaaaaaggacaacggccccgtgatcaagaagattaagtattacggcaacaaactgaacgcccatctggacatcaccgacgactaccccaacagcagaaacaaggtcgtgaagctgtccctgaagccctacagattcgacgtgtacctggacaatggcgtgtacaagttcgtgaccgtgaagaatctggatgtgatcaaaaaagaaaactactacgaagtgaatagcaagtgctatgaggaagctaagaagctgaagaagatcagcaaccaggccgagtttatcgcctccttctacaacaacgatctgatcaagatcaacggcgagctgtatagagtgatcggcgtgaacaacgacctgctgaaccggatcgaagtgaacatgatcgacatcacctaccgcgagtacctggaaaacatgaacgacaagaggccccccaggatcattaagacaatcgcctccaagacccagagcattaagaagtacagcacagacattctgggcaacctgtatgaagtgaaatctaagaagcaccctcagatcatcaaaaagggcgctcctccggcacaacctcccagccaacctcagcagcattatagcgaaggagagttggaggaagatgaagattctgatgacgcgcgcaaaaaacgcaaaaaaaaatagcaataaaggatcgtttattttcattggaagcgtgtgttggttttttgatcaggcgcgaaaaaatctcgccaacaagttgacgagataaacacggcattttgccttgttatagtagattctgtttccagagtactataaccacatttgcttcagcactgaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaaaaaatctcgccaacaagttgacgagataaacacggcattttgccttgttatagtagattctgtttccagagtactataaccgacaaaggtcaacaccactccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcaggaacccctagtgatggagttggccactccctctctgcgcgctcgctcgctcactgaggccgggcgaccaaaggtcgcccgacgcccgggctttgcccgggcggcctcagtgagcgagcgagcgcgc

SEQ ID NO: 141 - DNA, 19 bp, synthetic construct
  source 1..19: mol_type = other DNA
ctgagaattgggaacatgc

SEQ ID NO: 142 - DNA, 18 bp, synthetic construct
  source 1..18: mol_type = other DNA
catcggaaccttccaggg

SEQ ID NO: 143 - DNA, 30 bp, synthetic construct
  source 1..30: mol_type = other DNA
acaaatggtatcttaaggacctccaaggtg